(19) United States
(12) Patent Application Publication
(10) Pub. No.: US 2021/0230616 A1
(43) Pub. Date: Jul. 29, 2021

(54) METHODS FOR ISOLATING CELLS WITHOUT THE USE OF TRANSGENIC MARKER SEQUENCES

(71) Applicant: INSTITUTE OF GENETICS AND DEVELOPMENTAL BIOLOGY, CHINESE ACADEMY OF SCIENCES, Beijing (CN)

(72) Inventors: Caixia GAO, Beijing (CN); Rui ZHANG, Beijing (CN); Jinxing LIU, Beijing (CN); Aaron HUMMEL, St. Louis, MO (US); Zarir VAGHCHHIPAWALA, Ballwin, MO (US); Mathias LABS, Frontenac, MO (US)

(73) Assignee: INSTITUTE OF GENETICS AND DEVELOPMENTAL BIOLOGY, CHINESE ACADEMY OF SCIENCES, Beijing (CN)

(21) Appl. No.: 16/610,782

(22) PCT Filed: May 7, 2018
(86) PCT No.: PCT/CN2018/085829

§ 371 Date: Nov. 4, 2019

Related U.S. Application Data
(60) Provisional application No. 62/502,418, filed on May 5, 2017.

(30) Foreign Application Priority Data
Sep. 1, 2017 (CN) 201710778196.0

(51) Int. Cl. C12N 15/82

(52) U.S. Cl. CPC C12N 15/8213; C12N 2810/10; C12N 15/8274

(57) ABSTRACT

The present invention relates to methods for targeted editing in a plant, a plant cell or material, which is combined with the parallel introduction of a phenotypically selectable trait. Furthermore, methods are provided not comprising a step of introducing a transgenic selection marker sequence.

### TECHNICAL FIELD

The present invention relates to methods for targeted editing in a plant, a plant cell or material, which is combined with the parallel introduction of a phenotypically selectable trait. Furthermore, methods are provided not comprising a step of introducing a transgenic selection marker sequence. The methods comprise introducing a targeted modification at a first genomic target site to obtain a selectable phenotype which does not rely on the provision of an exogenous polynucleotide template, nor does it rely on the introduction of a double-stand break at the target site. Finally, the invention relates to the combination of specific method steps parallelizing transgenic marker-free selection and targeted editing at different genomic target site resulting in conferring a selectable or other phenotype enabling the isolation of plant material without a selection marker cassette to allow precision breeding comprising significantly reduced selection efforts for identifying a genotype of interest.

### BACKGROUND OF THE INVENTION

Precise modification of genetic information of eukaryotic cells is of high value for agricultural, pharmaceutical, and medical applications, but is also substantial for basic research. Genome engineering or editing describes the ability to make these defined genetic changes in targets with high precision. Targeted double strand breaks can, for example, be created by site-specific nucleases (SSNs) or recombinases in eukaryotic cells.

In plants, precision double strand break induction increases the frequency of homologous recombination (HR) events by 100× to 1000× (Puchta et al., Proc. Natl. Acad. Sci. USA 93:5055-5060, 1996). However, the downstream identification of modified cells and plants is a limitation to the routine implementation of gene editing as a breeding tool for plant improvement.

Plant breeding and developments in agricultural technology such as agrochemicals has/have made remarkable progress in increasing crop yields for over a century. However, plant breeders must constantly respond to many changes. Agricultural practices change, which creates the need for developing plants with genotypes carrying specific agronomic characteristics. Furthermore, target environments and the organisms within them are constantly changing. For example, fungal and insect pests continually evolve and overcome resistance of a plant of interest. New land areas are regularly being used for farming, exposing plants to altered growing conditions. Finally, consumer preferences and requirements change. Plant breeders therefore face the endless task of continually developing new crop varieties (Collard and Mackill, Philos. Trans. R. Soc. Lond. B. Biol. Sci. 2008 Feb. 12; 363(1491): 557-572).

To assist breeding strategies, selectable marker sequences or marker-assisted selection (MAS) strategies are thus needed having a diagnostic potential so that a genotype of interest can reliably determined. As disclosed in EP 2 342 337 B1, the development of diagnostic markers follows a process starting with the mapping of the genetic position of the gene(s) underlying a trait of interest, the identification of flanking markers, fine mapping of the gene(s) by identification of tightly linked markers, determination of the DNA marker sequences of the most linked markers, determination of the sequence variation at the marker loci between the parent lines used to map the target gene, development of simple PCR assays, test of predictive value in the genetic background (germplasm) of the plant material where a marker with diagnostic properties during screening or breeding will be tested. Said strategies are inherently laborious and thus cost intensive, as a marker of interest has to be present, or has to be inserted, at a suitable position within a genome of interest.

DNA marker technology can dramatically enhance the efficiency of plant breeding by allowing selection on the basis of easy to assay markers, instead of determining phenotypical traits. However, the development of such markers with diagnostic or screening properties and the effectiveness of applying these markers is often a laborious and time consuming process as detailed above. Currently, methods for detecting point mutations, e.g. SNPs, only can identify a limited number of such point mutations and detect a limited repertoire (Slade et al., Nat. Biotech. 23, 75-81).

Still, selectable marker genes play an important role in plant for transgenic and transplastomic plant research or crop development. Selectable marker genes are often used in combination with reporter genes, which reporter genes do not provide a cell with a selective advantage, but which reporter genes can be used to monitor transgenic events, or to manually separate transgenic material from non-transformed material.

An area that is advancing rapidly is the development of strategies for eliminating selectable marker genes to generate marker-free plants. The rationalization for creating marker-free plants has been discussed in detail in several reviews (Yoder and Goldsbrough, 1994; Ow, 2001; Hare and Chua, 2002). For commercialization of transgenic and non-transgenic plants it would simplify the regulatory process and improve consumer acceptance to remove gene sequences that are not serving a purpose in the final plant variety. Eliminating marker genes from the final plant would permit the use of experimental marker genes that have not undergone extensive biosafety evaluations or that may generate negative pleiotropic effects in the plants. Furthermore, it would permit the recycling of useful marker genes for recurrent transformation of transgenic plants if they were eliminated prior to the next round of transformation.

Transgenic selection marker genes can thus increase the efficiency of recovering plants regenerated from treated cells, but the introduction of transgenic sequencing into the plant genome is not always desirable. Furthermore, the elimination of transgenic marker genes after selection has been achieved is often very complicated.

Precision gene editing or genome engineering has evolved as one of the most important areas of genetic engineering allowing the targeted and site-directed manipulation of a genome of interest over the last years. An indispensable prerequisite for site-directed genome engineering are programmable nucleases, which can be used to break a nucleic acid of interest at a defined position to induce either a double-strand break (DSB) or one or more single-strand breaks. Alternatively, said nucleases can be chimeric or mutated variants, no longer comprising a nuclease function, but rather operating as recognition molecules in combination with another enzyme. Those nucleases or variants thereof are thus key to any gene editing or genome engineering approach. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas, Cpf1, CasX or CasY nucleases as part of the Clustered Regularly Interspaced Short Palindromic Repeats (CRISPR) system.

CRISPRs (Clustered Regularly Interspaced Short Palindromic Repeats) in their natural environment originally evolved in bacteria where the CRISPR system fulfils the role of an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a CRISPR effector protein to a target sequence in the viral genome. The CRISPR effector protein cleaves and thereby interferes with replication of the viral target. Over the last years, the CRISPR system has successfully been adapted for gene editing or genome engineering also in eukaryotic cells. Editing in animal cells and therapeutic applications for human beings are presently of significant research emphasis. The targeted modification of complex animal and also plant genomes still represents a demanding task.

A CRISPR system in its natural environment describes a molecular complex comprising at least one small and individual non-coding RNA in combination with a Cas nuclease or another CRISPR nuclease like a Cpf1 nuclease (Zetsche et al., “Cpf1 Is a Single RNA-Guides Endonuclease of a Class 2 CRISPR-Cas System”, Cell, 163, pp. 1-13, October 2015) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbial., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems code for a Cas9 nuclease as key enzyme for the interference step, which systems contain both a crRNA and also a trans-activating RNA (tracrRNA) as the guide motif These hybridize and form double-stranded (ds) RNA regions which are recognized by RNAse III and can be cleaved in order to form mature crRNAs. These then in turn associate with the Cas molecule in order to direct the nuclease specifically to the target nucleic acid region. Recombinant gRNA molecules can comprise both, the variable DNA recognition region and also the Cas interaction region, and can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al., “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex efficiently cleaves target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized.

Presently, for example, Type II systems relying on Cas9, or a variant or any chimeric form thereof, as endonuclease have been modified for genome engineering. Synthetic CRISPR systems consisting of two components, a guide RNA (gRNA) also called single guide RNA (sgRNA) and a non-specific CRISPR-associated endonuclease can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA (“single guide RNA” (sgRNA) or simply “gRNA”; Jinek et al., 2012, supra). The genomic target can be any ˜20 nucleotide DNA sequence, provided that the target is present immediately upstream of a PAM. The PAM sequence is of outstanding importance for target binding and the exact sequence is dependent upon the species of Cas9 and, for example, reads 5′ NGG 3′ or 5′ NAG 3′ (Standard IUPAC nucleotide code) (Jinek et al., 2012, supra) for a Streptococcus pyogenes derived Cas9. Using modified Cas nucleases, targeted single strand breaks can be introduced into a target sequence of interest. The combined use of such a Cas nickase with different recombinant gRNAs highly site specific DNA double strand breaks can be introduced using a double nicking system. Using one or more gRNAs can further increase the overall specificity and reduce off-target effects.

Once expressed, the Cas9 protein and the gRNA form a ribonucleoprotein complex through interactions between the gRNA “scaffold” domain and surface-exposed positively-charged grooves on Cas9. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

Recently, engineered CRISPR/Cpf1 systems in addition to CRISPR/Cas9 systems become more and more important for targeted genome engineering (see Zetsche et al., supra and EP 3 009 511 A2). The Type V system together with the Type II system belongs to the Class 2 CRISPR systems (Makarova and Koonin Methods. Mol. Biol., 2015, 1311:47-753). The Cpf1 effector protein is a large protein (about 1,300 amino acids) that contains a RuvC like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain (Chylinski, 2014; Makarova, 2015). Cpf1 effectors possess certain differences over Cas9 effectors, namely no requirement of additional trans-activating crRNAs (tracrRNA) for CRISPR array processing, efficient cleavage of target DNA by short T-rich PAMs (in contrast to Cas9, where the PAM is followed by a G-rich sequence), and the introduction of staggered DNA double strand breaks by Cpf1. Very recently, additional novel CRISPR-Cas systems based on CasX and CasY have been identified which due to the relatively small size of the effector protein are of specific interest for many gene editing or genome engineering approaches (Burstein et al., “New CRISPR-Cas systems from uncultivated microbes”, Nature, December 2016).

Still, the CRISPR systems per se lack the inherent capacity to create a point mutation at a desired position in a genome of interest in a target cell.

Genome engineering tools like CRISPR systems introducing a double-strand break (DSB) require a DSB repair mechanism. Said mechanisms have been divided into two major basic types, non-homologous end joining (NHEJ) and homologous recombination (HR). Homology based repair mechanisms in general are usually called homology-directed repair (HOR).

NHEJ is the dominant nuclear response in animals and plants which does not require homologous sequences, but is often error-prone and thus potentially mutagenic (Wyman C., Kanaar R. “DNA double-strand break repair: all's well that ends well”, Annu. Rev. Genet. 2006; 40, 363-83). Repair by HOR requires homology, but those HOR pathways that use an intact chromosome to repair the broken one, i.e., double-strand break repair and synthesis-dependent strand annealing, are highly accurate. In the classical DSB repair pathway, the 3′ ends invade an intact homologous template then serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Synthesis-dependent strand annealing is conservative, and results exclusively in non-crossover events.

This means that all newly synthesized sequences are present on the same molecule. Unlike the NHEJ repair pathway, following strand invasion and D loop formation in synthesis-dependent strand annealing, the newly synthesized portion of the invasive strand is displaced from the template and returned to the processed end of the non-invading strand at the other DSB end. The 3′ end of the non-invasive strand is elongated and ligated to fill the gap. There is a further pathway of HOR, called break-induced repair pathway not yet fully characterized. A central feature of this pathway is the presence of only one invasive end at a DSB that can be used for repair.

Therefore, introducing a targeted point mutation into a plant genome and utilizing said mutation is a challenging task at date. Furthermore, the potential of genome engineering using site-specific nucleases (SSNs) still faces the problem of selecting for the modifications introduced by said SSNs, particularly in case the genome of interest is a complex eukaryotic genome, like a plant genome, and the targeted modification has to be traced over selective rounds during breeding.

Despite the abundance of genome engineering (GE) possibilities available at date, most of said GE approaches aim at introducing a targeted modification of interest by one SSN comprising complex. The introduction of such a targeted modification into a plant germplasm for subsequent plant breeding is thus possible, yet the subsequent tracing of the targeted modification is cumbersome. If a selection marker, or a selection marker cassette is used to assist the selection and thus isolation of cells of potential interest, there is still the huge hurdle of removing such a marker cassette from the genome of a plant after successive rounds of crossing during breeding for achieving genotype/phenotype combination of interest.

At the same time, there is a great need in providing new methods suitable for plant breeding, wherein traits of interest, e.g., based on a modification of interest, an elite event, or a favorable property from a cultivar to be crossed-in, can be defined, created, or crossed-in during breeding. It is sometimes hard, or very time-consuming to screen for the propagation and presence of said traits of interest during the different steps of breeding.

Therefore, better methods are needed to isolate cells and plants, preferably methods that do not require genomic integration of a transgenic marker sequence for subsequent rounds of selection. Furthermore, there is a great need for selectable marker sequences, which can be created in a site-directed way with high precision and without introducing exogenous transgenic sequences for the purpose of selection and screening means. Finally, there is a huge need in defining new strategies assisting rapid breeding to stack traits of interest together into the germplasm during successive rounds of crossing and selection during breeding.

Therefore, it was an object underlying the present application to provide methods to isolate cells that have been treated with and edited by gene editing reagents by using an easy to screen phenotypically selectable trait. To this end, a targeted modification is made at a first gene to confer a selectable or other phenotype on the cell and its progeny refraining from introducing a transgenic selectable marker sequence. In parallel, a targeted modification is made at a second gene of interest that may or usually may not confer a phenotype on the cell. The cell and its progeny cells or plants can be isolated or regenerated from a background of untreated cells by applying a selection agent or other method that uses the phenotype conferred by the modification at the first gene to identify the cells that have undergone this first gene modification. Cells or plants with the targeted modification at the second gene of interest, which second modification represents the actual aim to be achieved, are identified from this population to provide faster and thus cheaper selection without the need of a transgenic selectable marker sequence present, or to be introduced, in a genome of interest.

### SUMMARY OF THE INVENTION

The above identified objects have been achieved as detailed herein by defining a strategy to parallelizing the site-directed introduction of a non-transgenic and phenotypically selectable modification together with the targeted introduction of a second site-directed modification of interest. Usually the second modification will have no opportunity for selection because the phenotype it confers will not be expressed or relevant in the process of generating the plants. So the purpose underlying the methods of the present invention is to use the first modification as a tool to enable selection. Compared to traditional strategies, the methods of the present invention have the advantage of not incorporating a transgenic marker gene. Compared to not using a selectable phenotype selectable with a corresponding selection agent, it has the advantage of increased efficiency by eliminating all or most untreated cells, which would otherwise comprise the majority the cells producing plants. By eliminating untreated cells not having undergone a targeted modification at a first plant genomic target site causing the expression of a phenotypically selectable trait, the number of plants that have to be produced is greatly reduced, and the number of plants that have to be molecularly screened for the second modification is greatly reduced. The methods according to the present invention thus significantly increase the efficiency of breeding and avoid labor-intensive steps.

Specifically, the above objects have been achieved by providing, in a first aspect, a method for isolating at least one modified plant cell or at least one modified plant tissue, organ, or whole plant comprising the at least one modified plant cell, without stably integrating a transgenic selectable marker sequence, the method comprising: (a) introducing at least one first targeted base modification into a first plant genomic target site of at least one plant cell to be modified, wherein the at least one targeted base modification causes expression of at least one phenotypically selectable trait; (b) introducing at least one second targeted modification into a second plant genomic target site of the at least one plant cell to be modified, wherein the at least one second targeted modification is introduced using at least one of a site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted base modification at the first plant genomic target site, and optionally by further selecting (ii) for the at least one second targeted modification in the second plant genomic target site.

In one embodiment according to the various aspects of the present invention, there is provided a method, wherein step (b) additionally comprises introducing a repair template to make a targeted sequence conversion or replacement at the at least second plant genomic target site.

In a further embodiment, the method according to the first aspect comprises a further step of (d) crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

In one embodiment, the at least one site-specific effector is temporarily or permanently linked to at least one base editing complex, wherein the base editing complex mediates the at least one first targeted base modification of step (a).

In a further embodiment, the at least one site-specific effector is selected from at least one of a nuclease, comprising a CRISPR nuclease, including Cas or Cpf1 nucleases, a TALEN, a ZFN, a meganuclease, an Argonaute nuclease, a restriction endonuclease, including FokI or a variant thereof, a recombinase, or two site-specific nicking endonucleases, or a base editor, or any variant or catalytically active fragment of the aforementioned effectors.

In yet a further embodiment, the at least one site-specific effector is a CRISPR-based nuclease, wherein the CRISPR-based nuclease comprises a site-specific DNA binding domain directing the at least one base editing complex, wherein the at least one CRISPR-based nuclease, or the nucleic acid sequence encoding the same, is selected from the group comprising (a) Cas9, including SpCas9, SaCas9, SaKKH-Cas9, VQR-Cas9, St1Cas9, (b) Cpf1, including AsCpf1, LbCpf1, FnCpf1, (c) CasX, or (d) CasY, or any variant or derivative of the aforementioned CRISPR-based nucleases, preferably wherein the at least one CRISPR-based nuclease comprises a mutation in comparison to the respective wild-type sequence so that the resulting CRISPR-based nuclease is converted to a single-strand specific DNA nickase, or to a DNA binding effector lacking all DNA cleavage ability.

In one embodiment, the at least one first targeted base modification according to the first aspect is made by at least one base editing complex comprising at least one base editor as component.

In one embodiment, the base editing complex comprises at least one cytidine deaminase, or a catalytically active fragment thereof.

In a further embodiment, the at least one first targeted base modification is a conversion of any nucleotide C, A, T, or G, to any other nucleotide.

In one embodiment according to the methods of the present invention, the base editing complex contains at least one of an APOBEC1 component, an UGI component, a XTEN component, or a PmCDA1 component. In a further embodiment, the at least one base editing complex comprises more than one component, and the at least two components are physically linked.

In one embodiment according to the methods of the present invention, the at least one base editing complex comprises more than one component, and the at least two components are provided as individual components.

In a further embodiment according to the methods of the present invention, the at least one component of the at least one base editing complex comprises at least one organelle localization signal to target the at least one base editing complex to a subcellular organelle. In one embodiment, the at least one organelle localization signal is a nuclear localization signal (NLS), in a further embodiment, the at least one organelle localization signal is a chloroplast transit peptide. In yet a further embodiment, the at least one organelle localization signal is a mitochondria transit peptide.

According to one embodiment of the methods of the present invention, the first plant genomic target site of the at least one plant cell is a genomic target site encoding at least one phenotypically selectable trait, wherein the at least one phenotypically selectable trait is a resistance/tolerance trait or a growth advantage trait, and wherein the at least one first targeted base modification at the first plant genomic target site of the at least one plant cell confers resistance/tolerance or a growth advantage towards a compound or trigger to be added to the at least one modified plant cell, tissue or plant, or a progeny thereof.

In one embodiment, the at least one phenotypically selectable trait of interest is or is encoded by at least one endogenous gene, or the at least one phenotypic trait of interest is or is encoded by at least one transgene, wherein the at least one endogenous gene or the at least one transgene encode(s) at least one phenotypic trait selected from the group consisting of resistance/tolerance to a phytotoxin, preferably a herbicide, inhibiting, damaging or killing cells lacking the at least one modification at the at least one phenotypic trait of interest, or wherein the at least one phenotypic trait is selected from the group consisting of boosters of cell division, growth rate, embryogenesis, or another phenotypically selectable property that provides an advantage to a modified cell, tissue, organ, or plant compared to an unmodified cell, tissue, organ, or plant.

In one embodiment, the at least one first plant genomic target site is at least one endogenous gene or a transgene encoding at least one phenotypically selectable trait selected from the group consisting of herbicide resistance/tolerance, wherein the herbicide resistance/tolerance is selected from the group consisting of resistance/tolerance to EPSPS-inhibitors, including glyphosate, resistance/tolerance to glutamine synthesis inhibitors, including glufosinate, resistance/tolerance to ALS- or AHAS-inhibitors, including imidazoline or sulfonylurea, resistance/tolerance to ACCase inhibitors, including aryloxyphenoxypropionate (FOP), resistance/tolerance to carotenoid biosynthesis inhibitors, including inhibitors of carotenoid biosynthesis at the phytoene desaturase step, inhibitors of 4-hydroxyphenyl-pyruvate-dioxygenase (HPPD), or inhibitors of other carotenoid biosynthesis targets, resistance/tolerance to cellulose inhibitors, resistance/tolerance to lipid synthesis inhibitors, resistance/tolerance to long-chain fatty acid inhibitors, resistance/tolerance to microtubule assembly inhibitors, resistance/tolerance to photosystem I electron diverters, resistance/tolerance to photosystem II inhibitors, including carbamate, triazines and triazinones, resistance/tolerance to PPO-inhibitors and resistance/tolerance to synthetic auxins, including dicamba (2,4-D, i.e., 2,4-dichlorophenoxyacetic acid).

In a further embodiment, the at least one phenotypically selectable trait is a phytotoxic resistance/tolerance trait, preferably a herbicide resistance/tolerance trait, and wherein the at least one first targeted base modification at the first plant genomic target site of the at least one plant cell to be modified confers resistance/tolerance for a phytotoxic compound, preferably a herbicide, said compound being an exogenous compound to be added to the at least one modified plant cell, tissue, organ, or whole plant, or a progeny thereof.

In one embodiment, the first plant genomic target site of the at least one plant cell is ALS. In another embodiment, the first plant genomic target site of the at least one plant cell is PPO. In yet another embodiment, the first plant genomic target site of the at least one plant cell is EPSPS, ALS, or PPO, and wherein the EPSPS, ALS or PPO comprises at least one nucleic acid conversion resulting in at least one corresponding amino acid conversion, wherein the at least one nucleic acid conversion is made by at least one base editor.

In one embodiment, the methods of the present invention comprises introduction of a targeted modification into the first plant genomic target site of the at least one plant cell, wherein the first plant genomic target site is ALS, and wherein the targeted modification occurs at the sequence encoding A122 in comparison to an ALS reference sequence according to SEQ ID NO:25, or at the sequence encoding P197 in comparison to an ALS reference sequence according to SEQ ID NO:25, or at the sequence encoding A205 in comparison to an ALS reference sequence according to SEQ ID NO:25, or at the sequence encoding D376 in comparison to an ALS reference sequence according to SEQ ID NO:25, or at the sequence encoding R377 in comparison to an ALS reference sequence according to SEQ ID NO:25. In still another embodiment, a targeted modification occurs at the sequence encoding W574 in comparison to an ALS reference sequence according to SEQ ID NO:25. According to one embodiment, a targeted modification occurs at the sequence encoding S653 in comparison to an ALS reference sequence according to SEQ ID NO:25. In one embodiment, a targeted modification occurs at the sequence encoding G654 in comparison to an ALS reference sequence according to SEQ ID NO:25.

In one embodiment of the methods of the present invention, the first plant genomic target site of the at least one plant cell is PPO, and a targeted modification occurs at the sequence encoding C215 in comparison to an PPO reference sequence according to SEQ ID NO:26. In another embodiment, a targeted modification occurs at the sequence encoding A220 in comparison to an PPO reference sequence according to SEQ ID NO:26. In a further embodiment, a targeted modification occurs at the sequence encoding G221 in comparison to an PPO reference sequence according to SEQ ID NO:26. In yet a further embodiment, wherein the first plant genomic target site of the at least one plant cell is PPO, a targeted modification occurs at the sequence encoding N425 in comparison to an PPO reference sequence according to SEQ ID NO:26, or at the sequence encoding Y426, or at the sequence encoding I475, in comparison to an PPO reference sequence according to SEQ ID NO:26.

In one embodiment according to the methods of the present invention, the first plant genomic target site of the at least one plant cell is EPSPS, and targeted modifications occur at the sequence encoding G101 and at G144, at the sequence encoding G101 and at A192, or at the sequence encoding T102 and at P106, all sequences in comparison to an EPSPS reference sequence according to SEQ ID NO:27.

Further combinations or additional modifications of targeted modifications of the first genomic target site are within the scope of the present invention.

In one embodiment of the methods of the present invention, the at least one phenotypically selectable trait is a visible phenotype that is useful in identifying or isolating at least one modified plant cell, tissue, organ or whole plant. The at least one phenotypically selectable trait can be a glossy phenotype, a golden phenotype, a growth advantage phenotype, or a pigmentation phenotype, or any other visually screenable phenotype.

In a second aspect according to the present invention, there is provided a method for isolating at least one modified plant cell or at least one modified plant tissue, organ, or whole plant comprising the at least one modified plant cell, without stably integrating a transgenic selectable marker sequence, the method comprising: (a) introducing at least one first targeted codon deletion modification into a first plant genomic target site of at least one plant cell to be modified using at least one first site-specific effector, comprising a nuclease, a recombinase, or a DNA modification reagent, wherein the at least one targeted codon deletion modification causes expression of at least one phenotypically selectable trait; (b) introducing at least one second targeted modification into a second plant genomic target site of the at least one plant cell to be modified, wherein the at least one second targeted modification is introduced using at least one second site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted codon deletion modification at the first plant genomic target site, and optionally by further selecting (ii) for the at least one second targeted modification in the second plant genomic target site, (d) optionally: crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

In a further aspect according to the present invention there is provided a method for isolating at least one modified plant cell or at least one modified tissue, organ, or whole plant comprising the at least one modified plant cell, without stably integrating a transgenic selectable marker sequence, the method comprising: (a) introducing at least one first targeted frameshift or deletion modification into a first plant genomic target site of at least one plant cell to be modified using at least one first site-specific effector, wherein the at least one targeted frameshift or deletion modification causes expression of at least one phenotypically selectable trait; (b) introducing at least one second targeted modification into a second plant genomic target site of the at least one plant cell to be modified, wherein the at least one second targeted modification is introduced using at least one second site-specific effector, comprising a nuclease, a recombinase, or a DNA modification reagent, to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or whole plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted frameshift or deletion modification at the first plant genomic target site, and optionally by further selecting (ii) for the at least one second targeted modification in the second plant genomic target site, (d) optionally: crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

In one embodiment according to the above aspects, preferably step (b) additionally comprises introducing a repair template to make a targeted sequence conversion or replacement at the at least one first and/or second plant genomic target site.

In a further embodiment, the at least one site-specific effector is selected from at least one of a CRISPR nuclease, including Cas or Cpf1 nucleases, a TALEN, a ZFN, a meganuclease, an Argonaute nuclease, a restriction endonuclease, including FokI or a variant thereof, a recombinase, or two site-specific nicking endonucleases, or any variant or catalytically active fragment of the aforementioned effectors.

In one embodiment according to the various aspects of the present invention, the at least one site-specific effector is a CRISPR-based nuclease, wherein the CRISPR-based nuclease comprises a site-specific DNA binding domain, wherein the at least one CRISPR-based nuclease, or the nucleic acid sequence encoding the same, is selected from the group comprising (a) Cas9, including SpCas9, SaCas9, SaKKH-Cas9, VQR-Cas9, St1Cas9, (b) Cpf1, including AsCpf1, LbCpf1, FnCpf1, (c) CasX, or (d) CasY, or any variant or derivative of the aforementioned CRISPR-based nucleases, optionally wherein the at least one CRISPR-based nuclease comprises a mutation in comparison to the respective wild-type sequence so that the resulting CRISPR-based nuclease is converted to a single-strand specific DNA nickase, or to a DNA binding effector lacking all DNA cleavage ability.

In a further embodiment according to the aspects of the present invention, the at least site-specific effector, or at least one component of a complex comprising the at least one site-specific effector, comprises at least one organelle localization signal to target the at least one base editing complex to a subcellular organelle, wherein the at least one organelle localization signal can be selected from a nuclear localization signal (NLS), a chloroplast transit peptide, or a mitochondria transit peptide.

In one embodiment of the above aspects, the first plant genomic target site of the at least one plant cell is a genomic target site encoding at least one phenotypically selectable trait, wherein the at least one phenotypically selectable trait is a resistance/tolerance trait or a growth advantage trait, and wherein the at least one first targeted base modification at the first plant genomic target site of the at least one plant cell confers resistance/tolerance or a growth advantage towards a compound or trigger to be added to the at least one modified plant cell, tissue or plant, or a progeny thereof.

In a further embodiment of the above aspects, the at least one phenotypically selectable trait of interest is or is encoded by at least one endogenous gene, or the at least one phenotypic trait of interest is or is encoded by at least one transgene, wherein the at least one endogenous gene or the at least one transgene encode(s) at least one phenotypic trait selected from the group consisting of resistance/tolerance to a phytotoxin, preferably a herbicide, inhibiting, damaging or killing cells lacking the at least one modification at the at least one phenotypic trait of interest, or wherein the at least one phenotypic trait is selected from the group consisting of boosters of cell division, growth rate, embryogenesis, or another phenotypically selectable property that provides an advantage to a modified cell, tissue, organ, or plant compared to an unmodified cell, tissue, organ, or plant.

In yet a further embodiment of the above aspects, the at least one first plant genomic target site is at least one endogenous gene or a transgene encoding at least one phenotypically selectable trait selected from the group consisting of herbicide resistance/tolerance, wherein the herbicide resistance/tolerance is selected from the group consisting of resistance/tolerance to EPSPS-inhibitors, including glyphosate, resistance/tolerance to glutamine synthesis inhibitors, including glufosinate, resistance/tolerance to ALS- or AHAS-inhibitors, including imidazoline or sulfonylurea, resistance/tolerance to ACCase inhibitors, including aryloxyphenoxypropionate (FOP), resistance/tolerance to carotenoid biosynthesis inhibitors, including inhibitors of carotenoid biosynthesis at the phytoene desaturase step, inhibitors of 4-hydroxyphenyl-pyruvate-dioxygenase (HPPD), or inhibitors of other carotenoid biosynthesis targets, resistance/tolerance to cellulose inhibitors, resistance/tolerance to lipid synthesis inhibitors, resistance/tolerance to long-chain fatty acid inhibitors, resistance/tolerance to microtubule assembly inhibitors, resistance/tolerance to photosystem I electron diverters, resistance/tolerance to photosystem II inhibitors, including carbamate, triazines and triazinones, resistance/tolerance to PPO-inhibitors and resistance/tolerance to synthetic auxins, including dicamba (2,4-dichlorophenoxyacetic acid).

In one embodiment of the above aspects, the at least one phenotypically selectable trait is a phytotoxic resistance/tolerance trait, preferably a herbicide resistance/tolerance trait, and the at least one first targeted codon deletion or frameshift or deletion modification at the first plant genomic target site of the at least one plant cell to be modified confers resistance/tolerance for a phytotoxic compound, preferably a herbicide, said compound being an exogenous compound to be added to the at least one modified plant cell, tissue, organ, or whole plant, or a progeny thereof.

In one embodiment according to the various aspects of the present invention, the first plant genomic target site of the at least one plant cell is a homolog of the PPX2L gene product from Amaranthus tuberculatus for the purpose of selection.

In one embodiment according to the various aspects of the present invention, the at least one first targeted base modification, targeted codon deletion, or targeted frameshift or deletion modification occurs at the position comparable to the G210 residue of the PPX2L gene product from Amaranthus tuberculatus according to SEQ ID NO:28.

In one embodiment according to the various aspects of the present invention, the at least one phenotypically selectable trait is a visible phenotype that is useful in identifying or isolating at least one modified plant cell, tissue, organ, or whole plant. The at least one phenotypically selectable trait according to the various aspects of the present invention can be a glossy phenotype, a golden phenotype, a growth advantage phenotype or a pigmentation phenotype, or any other visually screenable phenotype.

In one embodiment of the methods according to all aspects of the present invention, the at least one plant cell to be modified is preferably being derived from a plant selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

### BRIEF DESCRIPTION OF THE DRAWINGS

FIG. 1 (FIG. 1A to C) illustrates how the methods according to the present invention can be implemented for isolating cells of interest during selection, for example, for plant breeding and targeted selection strategies. FIG. 1A shows the treatment of cells with a base editor (BE), or a BE complex, and editing reagents, i.e. a site-specific effector comprising a site-specific nuclease (SSN) at two different genomic locations in parallel. Arrows indicate the target site, where the base editor (complex) and the site-specific effector will introduce two targeted site-specific modifications. FIG. 1B shows the result of the preceding step illustrated in FIG. 1A, i.e., the BE (complex) introduces a modified phenotype at a gene of interest, highlighted in white, whereas the site-specific effector introduces a targeted edit in a trait gene, highlighted in black. Therefore, the two distinct modifications within two different genomic target sites allow the isolation of plant cells or plants from treated cells. Plants can then be screened for an edit at a gene of interest, which is usually different from the modified phenotype used for screening purposes. FIG. 1C then shows the result after segregating plants to achieve a desired genotype. This desired genotype of interest comprises the targeted modification (black) introduced via a site-specific effector, but does no longer comprise the modified phenotype modification, the latter having been introduced for selection purposes, yet not as a genomic trait comprised by the genome of the resulting plant cell, tissue, organ or whole plant in this example.

FIG. 2 illustrates enhanced screening efficiency by co-editing TaALS S1 site.

FIG. 3 illustrates generation of herbicide resistant wheat by editing TaALS-P173.

FIG. 4 illustrates generation of herbicide resistant corn by editing ZmALS-P165.

FIG. 5 illustrates the sequence structure and herbicide resistant sites to be edited in corn.

FIG. 6 illustrates efficient editing of ZmALS-P197 and ZmALS-G654.

FIG. 7 illustrates the efficiency of converting ZmALS-P197 and ZmALS-G654 to desired herbicide resistance-conferring residues.

### SEQUENCES

SEQ ID NO: 1 is a nucleotide sequence of an APOBEC1 (rat cytidine deaminase)-XTEN linker (see, for example, Schellenberger et al., “A recombinant polypeptide extends the in vivo half-life of peptides and proteins in a tunable manner”, Nature Biotechnol. 27, 1186-1190 (2009))-nCas9(D10A)-UGI (uracil DNA glycosylase inhibitor)-NLS encoding construct, which was not codon optimized. The sequence includes a 3′ stop codon TAA.

SEQ ID NO: 2 is a nucleotide sequence of an APOBEC1-XTEN linker-nCas9(D10A)-UGI-NLS encoding construct, which was codon optimized for use in cereal plants. The sequence includes a 3′ stop codon TAG.

SEQ ID NO: 3 represents an exemplary protospacer sequence for Zm_ALS1&2_P197S/L/F for base editing for a B73 reference genotype. The position is based on the coordinates of the residue in the Arabidopsis ALS homolog. The sequence applies for a SpCas9-derived (Streptococcus pyogenes Cas9-derived) based editor.

SEQ ID NO: 4 represents an exemplary protospacer sequence for Zm_ALS1&2_P197S/L/F for base editing for a B73 reference genotype. The position is based on the coordinates of the residue in the Arabidopsis ALS homolog. The sequence applies for a SaKKH-BE3-derived based editor (Staphylococcus aureus Cas9 (SaCas9)-derived mutant of SaCas9 with a relaxed PAM specificity).

SEQ ID NO: 5 represents an exemplary protospacer sequence for Zm_ALS1&2_P197S/L/F for base editing for a B73 reference genotype. The position is based on the coordinates of the residue in the Arabidopsis ALS homolog. The sequence applies for a VQR-BE3-derived based editor (Staphylococcus aureus Cas9 (SaCas9)-derived mutant of SaCas9 with a different PAM specificity).

SEQ ID NO: 6 represents an exemplary protospacer sequence for Zm_ALS1&2_S653N for base editing for a B73 reference genotype. The position is based on the coordinates of the residue in the Arabidopsis ALS homolog. The sequence applies for a SpCas9-derived based editor.

SEQ ID NO: 7 represents an exemplary protospacer sequence for Zm_PPO_A220_&_G221 for base editing for a B73 reference genotype. The position is based on the coordinates of the residue in the Arabidopsis PPO homolog. The sequence applies for a SpCas9-derived based editor.

SEQ ID NO: 8 represents an exemplary protospacer sequence for Zm_PPO_A220_&_G221 for base editing for a B73 reference genotype. The position is based on the coordinates of the residue in the Arabidopsis PPO homolog. The sequence applies for a SaKKH-BE3-derived based editor.

SEQ ID NO: 9 represents an exemplary protospacer sequence for Zm_PPO_A220_&_G221 for base editing for a B73 reference genotype. The position is based on the coordinates of the residue in the Arabidopsis PPO homolog. The sequence applies for a VQR-BE3-derived based editor.

SEQ ID NO: 10 represents an exemplary protospacer sequence for Zm_PPO_C215 for base editing for base editing for a B73 reference genotype. The position is based on the coordinates of the residue in the Arabidopsis PPO homolog. The sequence applies for a SpCas9-derived based editor.

SEQ ID NO: 11 represents an exemplary protospacer sequence for Zm_PPO_C215 for base editing for base editing for a B73 reference genotype. The position is based on the coordinates of the residue in the Arabidopsis PPO homolog. The sequence applies for a SaKKH-BE3-derived based editor.

SEQ ID NO: 12 represents an exemplary protospacer sequence for Zm_PPO_C215 for base editing for base editing for a B73 reference genotype. The position is based on the coordinates of the residue in the Arabidopsis PPO homolog. The sequence applies for a SaKKH-BE3-derived based editor.

SEQ ID NO: 13 represents an exemplary protospacer sequence for Zm_PPO_C215 for base editing for base editing for a B73 reference genotype. The position is based on the coordinates of the residue in the Arabidopsis PPO homolog. The sequence applies for a VQR-BE3-derived based editor.

SEQ ID NO: 14 is a nucleotide sequence of an APOBEC1-XTEN linker-CasX1-UGI-NLS encoding construct, which was codon optimized. The sequence includes a 3′ stop codon TAG.

SEQ ID NO: 15 is a nucleotide sequence of an APOBEC1-XTEN linker-AsCpf1(R1226A) (Acidaminococcus sp. Cpf1 with R1226A mutation)-UGI-NLS encoding construct, which was codon optimized. The sequence includes a 3′ stop codon TAG.

SEQ ID NO: 16 is a nucleotide sequence of a construct encoding NLS-dCas9-NLS-Linker-PmCDA1 (activation-induced cytidine deaminase (AID) ortholog PmCDA1 from sea lamprey, see Nishida et al. (Science 2016, vol. 353, issue 6305, aaf8729))-UGI. The sequence includes a 3′ stop codon TAG.

SEQ ID NO: 17 is a nucleotide sequence encoding an exemplary Cas9 nickase n(i)Cas9 (D10A).

SEQ ID NO: 18 is a nucleotide sequence encoding an exemplary CasX.

SEQ ID NO: 19 is a nucleotide sequence encoding an exemplary AsCpf1 (R1226A).

SEQ ID NO: 20 is a nucleotide sequence encoding an exemplary APOBEC1.

SEQ ID NO: 21 is a nucleotide sequence encoding an exemplary UGI.

SEQ ID NO: 22 is a nucleotide sequence encoding an exemplary PmCDA1.

SEQ ID NO: 23 represents an exemplary protospacer sequence for Zm_PPO_N425_&Y426 for base editing for base editing for a B73 reference genotype. The position is based on the coordinates of the residue in the Arabidopsis PPO homolog. The sequence applies for a VQR-BE3-derived based editor.

SEQ ID NO: 24 is a sequence of Acidaminococcus sp BV3L6 Cpf1 (AsCpf1), UniProtKB/Swiss-Prot identifier: U2UMQ6.1.

SEQ ID NO: 25 is a sequence of acetolactate synthase (ALS) (chloroplastic) from Arabidopsis thaliana, GenBank: AAW70386.

SEQ ID NO: 26 is a sequence of Arabidopsis thaliana protoporphyrinogen oxidase (PPO).

SEQ ID NO: 27 is a sequence of Arabidopsis thaliana 5-enolpyruvylshikimate-3-phosphate synthase (EPSPS), mature protein after chloroplast transit peptide removal; NCBI accession AAY25438.

SEQ ID NO: 28 is a sequence of Amaranthus tuberculatus mitochondrial protoporphyrinogen oxidase (PPX2L), cf. NCBI accession DQ386114.

Definitions:

It must be noted that, as used herein, the singular forms “a” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a” “an” and “the” do not denote a limitation of quantity, but rather denote the presence of “at least one” of the referenced item. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up 5 to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The term “catalytically active fragment” as used herein referring to amino acid sequences denotes the core sequence derived from a given template amino acid sequence, or a nucleic acid sequence encoding the same, comprising all or part of the active site of the template sequence with the proviso that the resulting catalytically active fragment still possesses the activity characterizing the template sequence, for which the active site of the native enzyme or a variant thereof is responsible. Said modifications are suitable to generate less bulky amino acid sequences still having the same activity as a template sequence making the catalytically active fragment a more versatile or more stable tool being sterically less demanding.

“Complementary” or “complementarity” as used herein describes the relationship between two DNA, two RNA, or, regarding hybrid sequences according to the present invention, between an RNA and a DNA nucleic acid region. Defined by the nucleobases of the DNA or RNA, two nucleic acid regions can hybridize to each other in accordance with the lock-and-key model. To this end the principles of Watson-Crick base pairing have the basis adenine and thymine/uracil as well as guanine and cytosine, respectively, as complementary bases apply. Furthermore, also non-Watson-Crick pairing, like reverse-Watson-Crick, Hoogsteen, reverse-Hoogsteen and Wobble pairing are comprised by the term “complementary” as used herein as long as the respective base pairs can build hydrogen bonding to each other, i.e., two different nucleic acid strands can hybridize to each other based on said complementarity.

The term “construct”, especially “genetic construct” or “recombinant construct” or “expression construct” as used herein refers to a construct comprising, inter alfa, plasmids or plasmid vectors, cosmids, artificial yeast chromosomes or bacterial artificial chromosomes (YACs and BACs), phagemides, bacterial phage based vectors, an expression cassette, isolated single-stranded or double-stranded nucleic acid sequences, comprising DNA and RNA sequences, or amino acid sequences, viral vectors, including modified viruses, and a combination or a mixture thereof, for introduction or transformation, transfection or transduction into a target cell or plant, plant cell, tissue, organ or material according to the present disclosure. A recombinant construct according to the present invention can comprise an effector domain, either in the form of a nucleic acid or an amino acid sequence, wherein an effector domain represents a molecule, which can exert an effect in a target cell and includes a transgene, an single-stranded or double-stranded RNA molecule, including a guideRNA, a miRNA, a single or duplexed CRISPR tracr/crRNA, or an siRNA, or an amino acid sequences, including, inter alia, an enzyme or a catalytically active fragment thereof, a binding protein, an antibody, a transcription factor, a nuclease, preferably a site specific nuclease, and the like. Furthermore, the recombinant construct can comprise regulatory sequences and/or localization sequences. The recombinant construct can be integrated into a vector, including a plasmid vector, and/or it can be present isolated from a vector structure, for example, in the form of a polypeptide sequence or as a non-vector connected single-stranded or double-stranded nucleic acid. After its introduction, e.g. by transformation, the genetic construct can either persist extrachromosomally, i.e. non integrated into the genome of the target cell, for example in the form of a double-stranded or single-stranded DNA, a double-stranded or single-stranded RNA or as an amino acid sequence. Alternatively, the genetic construct, or parts thereof, according to the present disclosure can be stably integrated into the genome of a target cell, including the nuclear genome or further genetic elements of a target cell, including the genome of plastids like mitochondria or chloroplasts. The term “plasmid vector” as used in this connection refers to a genetic construct originally obtained from a plasmid.

The term “delivery construct” or “delivery vector” as used herein refers to any biological or chemical means used as a cargo for transporting a nucleic acid, including a hybrid nucleic acid comprising RNA and DNA, and/or an amino acid sequence of interest into a target cell, preferably a eukaryotic cell. The term delivery construct or vector as used herein thus refers to a means of transport to deliver a genetic or a recombinant construct according to the present disclosure into a target cell, tissue, organ or an organism. A vector can thus comprise nucleic acid sequences, optionally comprising sequences like regulatory sequences or localization sequences for delivery, either directly or indirectly, into a target cell of interest or into a plant target structure in the desired cellular compartment of a plant. A vector can also be used to introduce an amino acid sequence or a ribonucleo-molecular complex into a target cell or target structure. Usually, a vector as used herein can be a plasmid vector. Furthermore, according to certain preferred embodiments according to the present invention, a direct introduction of a construct or sequence or complex of interest is conducted. The term direct introduction implies that the desired target cell or target structure containing a DNA target sequence to be modified according to the present disclosure is directly transformed or transduced or transfected into the specific target cell of interest, where the material delivered with the delivery vector will exert its effect. The term indirect introduction implies that the introduction is achieved into a structure, for example, cells of leaves or cells of organs or tissues, which do not themselves represent the actual target cell or structure of interest to be transformed, but those structures serve as basis for the systemic spread and transfer of the vector, preferably comprising a genetic construct according to the present disclosure to the actual target structure, for example, a meristematic cell or tissue, or a stem cell or tissue. In case the term vector is used in the context of transfecting amino acid sequences and/or nucleic sequences, including hybrid nucleic acid sequences, into a target cell the term vector implies suitable agents for peptide or protein transfection, like for example ionic lipid mixtures, cell penetrating peptides (CPPs), or particle bombardment. In the context of the introduction of nucleic acid material, the term vector cannot only imply plasmid vectors but also suitable carrier materials which can serve as basis for the introduction of nucleic acid and/or amino acid sequence delivery into a target cell of interest, for example by means of particle bombardment. Said carrier material comprises, inter alia, gold or tungsten particles. Finally, the term vector also implies the use of viral vectors for the introduction of at least one genetic construct according to the present disclosure like, for example, modified viruses for example derived from the following virus strains: adenoviral or adeno-associated viral (AAV) vectors, lentiviral vectors, herpes simplex virus (HSV-1), vaccinia virus, Sendai virus, Sindbis virus, Semliki forest alphaviruses, Epstein-Barr-Virus (EBV), Maize Streak Virus (MSV), Barley Stripe Mosaic Virus (BSMV), Brome Mosaic virus (BMV, accession numbers: RNA 1: X58456; RNA2: X58457; RNA3: X58458), Maize stripe virus (MSpV), Maize rayado fino virus (MYDV), Maize yellow dwarf virus (MYDV), Maize dwarf mosaic virus (MDMV), positive strand RNA viruses of the family Benyviridae, e.g., Beet necrotic yellow vein virus (accession numbers: RNA 1: NC_003514; RNA2: NC_003515; RNA3: NC_003516; RNA4: NC_003517) or of the family Bromoviridae, e.g., viruses of the genus Alfalfa mosaic virus (accession numbers: RNA1: NC_001495; RNA2: NC_002024; RNA3: NC_002025) or of the genus Bromovirus, e.g., BMV (supra), or of the genus Cucumovirus, e.g., Cucumber mosaic virus (accession numbers: RNA1: NC_002034; RNA2: NC_002035; RNA3: NC_001440), or of the genus Oleavirus, dsDNA viruses of the family Caulimoviridae, particularly of the family Badnavirus or Caulimovirus, e.g., different Banana streak viruses (e.g., accession numbers: NC_007002, NC_015507, NC_006955 or NC_003381) or Cauliflower mosaic virus (accession number: NC_001497), or viruses of the genus Cavemovirus, Petuvirus, Rosadnavirus, Solendovirus, Soymovirus or Tungrovirus, positive strand RNA viruses of the family Closteroviridae, e.g., of the genus Ampelovirus, Crinivirus, e.g., Lettuce infectious yellows virus (accession numbers: RNA 1: NC_003617; RNA2: NC_003618) or Tomato chlorosis virus (accession numbers: RNA 1: NC_007340; RNA2: NC_007341), Closterovirus, e.g., Beet yellows virus (accession number: NC_001598), or Velarivirus, single-stranded DNA (+/−) viruses of the family Geminiviridae, e.g., viruses of the family Becurtovirus, Begomovirus, e.g., Bean golden yellow mosaic virus, Tobacco curly shoot virus, Tobacco mottle leaf curl virus, Tomato chlorotic mottle virus, Tomato dwarf leaf virus, Tomato golden mosaic virus, Tomato leaf curl virus, Tomato mottle virus, or Tomato yellow spot virus, or Geminiviridae of the genus Curtovirus, e.g., Beet curly top virus, or Geminiviridae of the genus Topocuvirus, Turncurtvirus or Mastrevirus, e.g., Maize streak virus (supra), Tobacco yellow dwarf virus, Wheat dwarf virus, positive strand RNA viruses of the family Luteoviridae, e.g., of the genus Luteovirus, e.g., Barley yellow dwarf virus-PAV (accession number: NC_004750), or of the genus Polerovirus, e.g., Potato leafroll virus (accession number: NC_001747), single-stranded DNA viruses of the family Nanoviridae, comprising the genus Nanovirus or Babuvirus, double-stranded RNA viruses of the family Partiviridae, comprising inter alia the families Alphapartitivirus, Betapartitivirus or Deltapartitivirus, viroids of the family Pospiviroidae, positive strand RNA viruses of the family Potyviridae, e.g., comprising the genus Brambyvirus, Bymovirus, Ipomovirus, Macluravirus, Poacevirus, e.g., Triticum mosaic virus (accession number: NC_012799), or Potyviridae of the genus Potyvirus, e.g., Beet mosaic virus (accession number: NC_005304), Maize dwarf mosaic virus (accession number: NC_003377), Potato virus Y (accession number: NC_001616), or Zea mosaic virus (accession number: NC_018833), or Potyviridae of the genus Tritimovirus, e.g., Brome streak mosaic virus (accession number: NC_003501) or Wheat streak mosaic virus (accession number: NC_001886), single-stranded RNA viruses of the family Pseudoviridae, e.g., of the genus Pseudovirus, or Sirevirus, double-stranded RNA viruses of the family Reoviridae, e.g., Rice dwarf virus (accession numbers: RNA1: NC_003773; RNA2: NC_003774; RNA3: NC_003772; RNA4: NC_003761; RNAS: NC_003762; RNA6: NC_003763; RNA7: NC_003760; RNAB: NC_003764; RNA9: NC_003765; RNA10: NC_003766; RNA11: NC_003767; RNA 12: NC_003768), positive strand RNA viruses of the family Tombusviridae, e.g., comprising the genus Alphanecrovirus, Aureusvirus, Betanecrovirus, Carmovirus, Dianthovirus, Gallantivirus, Macanavirus, Machlomovirus, Panicovirus, Tombusvirus, Umbravirus oder Zeavirus, e.g., Maize necrotic streak virus (accession number: NC_007729), or positive strand RNA viruses of the family Virgaviridae, e.g., viruses of the genus Furovirus, Hordeivirus, e.g., Barley stripe mosaic virus (accession numbers: RNA1: NC_003469; RNA2: NC_003481; RNA3: NC_003478), or of the genus Pecluvirus, Pomovirus, Tobamovirus or Tobravirus, e.g., Tobacco rattle virus (accession numbers: RNA1: NC_003805; RNA2: NC_003811), as well as negative strand RNA viruses of the order Mononegavirales, particularly of the family Rhabdoviridae, e.g., Barley yellow striate mosaic virus (accession number: KM213865) or Lettuce necrotic yellows virus (accession number/specimen: NC_007642/AJ867584), positive strand RNA viruses of the order Picornavirales, particularly of the family Secoviridae, e.g., of the genus Comovirus, Fabavirus, Nepovirus, Cheravirus, Sadwavirus, Sequivirus, Torradovirus, or Waikavirus, positive strand RNA viruses of the order Tymovirales, particularly of the family Alphaflexiviridae, e.g., viruses of the genus Allexivirus, Lolavirus, Mandarivirus, or Potexvirus, Tymovirales, particularly of the family Betaflexiviridae, e.g., viruses of the genus Capillovirus, Carlavirus, Citrivirus, Foveavirus, Tepovirus, or Vitivirus, positive strand RNA viruses of the order Tymovirales, particularly of the family Tymoviridae, e.g., viruses of the order Maculavirus, Marafivirus, or Tymovirus, and bacterial vectors, like for example Agrobacterium spp., like for example Agrobacterium tumefaciens. Finally, the term vector also implies suitable chemical transport agents for introducing linear nucleic acid sequences (single- or double-stranded), or amino sequences, or a combination thereof into a target cell combined with a physical introduction method, including polymeric or lipid-based delivery constructs.

Suitable delivery constructs or vectors thus comprise biological means for delivering nucleotide sequences into a target cell, including viral vectors, Agrobacterium spp., or chemical delivery constructs, including nanoparticles, e.g., mesoporous silica nanoparticles (MSNPs), cationic polymers, including PEI (polyethylenimine) polymer based approaches or polymers like DEAE-dextran, or non-covalent surface attachment of PEI to generate cationic surfaces, lipid or polymeric vesicles, or combinations thereof. Lipid or polymeric vesicles may be selected, for example, from lipids, liposomes, lipid encapsulation systems, nanoparticles, small nucleic acid-lipid particle formulations, polymers, and polymersomes.

The term “derivative” or “descendant” or “progeny” as used herein in the context of a prokaryotic or a eukaryotic cell, preferably an animal cell and more preferably a plant or plant cell or plant material according to the present disclosure relates to the descendants of such a cell or material which result from natural reproductive propagation including sexual and asexual propagation. It is well known to the person having skill in the art that said propagation can lead to the introduction of mutations into the genome of an organism resulting from natural phenomena which results in a descendant or progeny, which is genomically different to the parental organism or cell, however, still belongs to the same genus/species and possesses mostly the same characteristics as the parental recombinant host cell. Such derivatives or descendants or progeny resulting from natural phenomena during reproduction or regeneration are thus comprised by the term of the present disclosure. Furthermore, the term “derivative” can imply, in the context of a substance or molecule rather than referring to a cell or organism, directly or by means of modification indirectly obtained from another. This might imply a nucleic acid sequence derived from a cell or a plant metabolite obtained from a cell or material. These terms, therefore, do not refer to any arbitrary derivative, descendant or progeny, but rather to a derivative, or descendant or progenitor phylogenetically associated with, i.e., based on, a parent cell or virus or a molecule thereof, whereas this relationship between the derivative, descendant or progeny and the “parent” is clearly inferable by a person skilled in the art.

Furthermore, the terms “derived”, “derived from”, or “derivative” as used herein in the context of a biological sequence (nucleic acid or amino acid) or a molecule or a complex imply that the respective sequence is based on a reference sequence, for example from the sequence listing, or a database accession number, or the respective scaffold structure, i.e., originating from said sequence, whereas the reference sequence can comprise more sequences, e.g., the whole genome or a full polyprotein encoding sequence, of a virus, whereas the sequence “derived from” the native sequence may only comprise one isolated fragment thereof, or a coherent fragment thereof. In this context, a cDNA molecule or a RNA can be said to be “derived from” a DNA sequence serving as molecular template. The skilled person can thus easily define a sequence “derived from” a reference sequence, which will, by sequence alignment on DNA or amino acid level, have a high identity to the respective reference sequence and which will have coherent stretches of DNA/amino acids in common with the respective reference sequence (>75% query identity for a given length of the molecule aligned provided that the derived sequence is the query and the reference sequence represents the subject during a sequence alignment). The skilled person can thus clone the respective sequences based on the disclosure provided herein by means of polymerase chain reactions and the like into a suitable vector system of interest, or use a sequence as vector scaffold. The term “derived from” is thus no arbitrary sequence, but a sequence corresponding to a reference sequence it is derived from, whereas certain differences, e.g., certain mutations naturally occurring during replication of a recombinant construct within a host cell, cannot be excluded and are thus comprised by the term “derived from”. Furthermore, several sequence stretches from a parent sequence can be concatenated in a sequence derived from the parent. The different stretches will have high or even 100% homology to the parent sequence. The skilled person is well aware of the fact that a sequence of the artificial molecular complexes according to the present invention when provided or partially provided as nucleic acid sequence will then be transcribed and optionally translated in vivo and will possibly be further digested and/or processed within a host cell (cleavage of signal peptides, endogenous biotinylation etc.) so that the term “derived from” indicates a correlation to the sequence originally used according to the disclosure of the present invention.

As used herein, “fusion” can refer to a protein and/or nucleic acid comprising one or more non-native sequences (e.g., moieties). A fusion can be at the N-terminal or C-terminal end of the modified protein, or both, or within the molecule as separate domain. For nucleic acid molecules, the fusion molecule can be attached at the 5′ or 3′ end, or at any suitable position in between. A fusion can be a transcriptional and/or translational fusion. A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-specific effector or base editor (e.g., a nuclear localization signal (NLS) for targeting to the nucleus, a mitochondrial localization signal for targeting to the mitochondria, a chloroplast localization signal for targeting to a chloroplast, an 15 endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-25 galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles, or nanodiamonds, optionally linked to a nanoparticle Any optical or magnetic property or characteristic of the nanoparticle(s) can be detected. A fusion can comprise a helicase, a nuclease (e.g., FokI), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long non-coding RNA, a DNA repair protein (e.g., a protein involved in repair of either single and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

The term “genetically modified” or “genetic manipulation” or “genetic(ally) manipulated” is used in a broad sense herein and means any modification of a nucleic acid sequence or an amino acid sequence, a target cell, tissue, organ or organism, which is accomplished by human intervention, either directly or indirectly, to influence the endogenous genetic material or the transciptome or the proteinome of a target cell, tissue, organ or organism to modify it in a purposive way so that it differs from its state as found without human intervention. The human intervention can either take place in vitro or in vivo/in planta, or also both. Further modifications can be included, for example, one or more point mutation(s), e.g. for targeted protein engineering or for codon optimization, deletion(s), and one or more insertion(s) or deletion(s) of at least one nucleic acid or amino acid molecule (including also homologous recombination), modification of a nucleic acid or an amino acid sequence, or a combination thereof. The terms shall also comprise a nucleic acid molecule or an amino acid molecule or a host cell or an organism, including a plant or a plant material thereof which is/are similar to a comparable sequence, organism or material as occurring in nature, but which have been constructed by at least one step of purposive manipulation. A “targeted genetic manipulation” or “targeted (base) modification” as used herein is thus the result of a “genetic manipulation”, which is effected in a targeted way, i.e. at a specific position in a target cell and under the specific suitable circumstances to achieve a desired effect in at least one cell, preferably a plant cell, to be manipulated, wherein the term implies that the sequence to be targeted and the corresponding modification are based on preceding sequence considerations so that the resulting modification can be planned in advance, e.g., based on available sequence information of a target site in the genome of a cell and/or based on the information of the target specificity (recognition or binding properties of a nucleic acid or an amino acid sequence, complementary base pairing and the like) of a molecular tool of interest.

The term “genome” refers to the entire complement of genetic material (genes and non-coding sequences) that is present in each cell of an organism, or virus or organelle; and/or a complete set of chromosomes inherited as a (haploid) unit from one parent. The term “particle bombardment” as used herein, also named “biolistic transfection or “microparticle-mediated gene transfer”, refers to a physical delivery method for transferring a coated microparticle or nanoparticle comprising a nucleic acid or a genetic construct of interest into a target cell or tissue. The micro or nanoparticle functions as projectile and is fired on the target structure of interest under high pressure using a suitable device, often called gene-gun. The transformation via particle bombardment uses a microprojectile of metal covered with the gene of interest, which is then shot onto the target cells using an equipment known as “gene gun” (Sandford et al. 1987) at high velocity fast enough (1500 km/h) to penetrate the cell wall of a target tissue, but not harsh enough to cause cell death. For protoplasts, which have their cell wall entirely removed, the conditions are different logically. The precipitated nucleic acid or the genetic construct on the at least one microprojectile is released into the cell after bombardment, and integrated into the genome. The acceleration of microprojectiles is accomplished by a high voltage electrical discharge or compressed gas (helium).

Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

The terms “genome editing” and “genome engineering” are used interchangeably herein and refer to strategies and techniques for the targeted, specific modification of any genetic information or genome of a living organism. As such, the terms comprise gene editing, but also the editing of regions other than gene encoding regions of a genome. It further comprises the editing or engineering of the nuclear (if present) as well as other genetic information of a cell. Furthermore, the terms “genome editing” and “genome engineering” also comprise an epigenetic editing or engineering, i.e., the targeted modification of, e.g., methylation, histone modification or of non-coding RNAs possibly causing heritable changes in gene expression.

“Germplasm”, as used herein, is a term used to describe the genetic resources, or more precisely the DNA of an organism and collections of that material. In breeding technology, the term germplasm is used to indicate the collection of genetic material from which a new plant or plant variety can be created.

The terms “guide RNA”, “gRNA” or “single guide RNA” or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. The tracr and the crRNA moiety thus do not necessarily have to be present on one covalently attached RNA molecule, yet they can also be comprised by two individual RNA molecules, which can associate or can be associated by non-covalent or covalent interaction to provide a gRNA according to the present disclosure. The terms “gDNA” or “sgDNA” or “guide DNA” are used interchangeably herein and either refer to a nucleic acid molecule interacting with an Argonaute nuclease. Both, the gRNAs and gDNAs as disclosed herein are termed “guiding nucleic acids” or “guide nucleic acids” due to their capacity to interacting with a site-specific nuclease and to assist in targeting said site-specific nuclease to a genomic target site.

As used herein, the terms “mutation” and “modification” are used interchangeably to refer to a deletion, insertion, addition, substitution, edit, strand break, and/or introduction of an adduct in the context of nucleic acid manipulation in vivo or in vitro. A deletion is defined as a change in a nucleic acid sequence in which one or more nucleotides is absent. An insertion or addition is that change in a nucleic acid sequence which has resulted in the addition of one or more nucleotides. A “substitution” or edit results from the replacement of one or more nucleotides by a molecule which is a different molecule from the replaced one or more nucleotides. For example, a nucleic acid may be replaced by a different nucleic acid as exemplified by replacement of a thymine by a cytosine, adenine, guanine, or uridine. Pyrimidine to pyrimidine (e.g., C to Tor T to C nucleotide substitutions) or purine to purine (e.g., G to A or A to G nucleotide substitutions) are termed transitions, whereas pyrimidine to purine or purine to pyrimidine (e.g., G to T or G to C or A to T or A to C) are termed transversions. Alternatively, a nucleic acid may be replaced by a modified nucleic acid as exemplified by replacement of a thymine by thymine glycol. Mutations may result in a mismatch. The term mismatch refers to a non-covalent interaction between two nucleic acids, each nucleic acid residing on a different nucleotide sequence or nucleic acid molecule, which does not follow the base-pairing rules. For example, for the partially complementary sequences 5′-AGT-3′ and 5′-AAT-3′, a G-A mismatch (a transition) is present.

The terms “nucleotide” and “nucleic acid” with reference to a sequence or a molecule are used interchangeably herein and refer to a single or double-stranded DNA or RNA of natural or synthetic origin. The term nucleotide sequence is thus used for any DNA or RNA sequence independent of its length, so that the term comprises any nucleotide sequence comprising at least one nucleotide, but also any kind of larger oligonucleotide or polynucleotide. The term(s) thus refer to natural and/or synthetic deoxyribonucleic acids (DNA) and/or ribonucleic acid (RNA) sequences, which can optionally comprise synthetic nucleic acid analoga. A nucleic acid according to the present disclosure can optionally be codon optimized. “Codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. Nucleic acid sequences according to the present application can carry specific codon optimization for the following non limiting list of organisms: Hordeum vulgare, Sorghum bicolor, Secale cereale, Saccharum officinarium, Zea mays, Setaria italic, Oryza sativa, Oryza minuta, Oryza australiensis, Oryza alta, Triticum aestivum, Triticum durum, Triticale, Hordeum bulbosum, Brachypodium distachyon, Hordeum marinum, Aegilops tauschii, Malus domestica, Beta vulgaris, Helianthus annuus, Daucus glochidiatus, Daucus pusillus, Daucus muricatus, Daucus carota, Eucalyptus grandis, Erythranthe guttata, Genlisea aurea, Nicotiana sylvestris, Nicotiana tabacum, Nicotiana tomentosiformis, Nicotiana benthamiana, Solanum lycopersicum, Solanum tuberosum, Coffea canephora, Vitis vinifera, Cucumis sativus, Marus notabilis, Arabidopsis thaliana, Arabidopsis lyrata, Arabidopsis arenosa, Crucihimalaya himalaica, Crucihimalaya wallichii, Cardamine flexuosa, Lepidium virginicum, Capsella bursa-pastoris, Olmarabidopsis pumila, Arabis hirsuta, Brassica napus, Brassica oleracea, Brassica rapa, Brassica juncacea, Brassica nigra, Raphanus sativus, Eruca vesicaria sativa, Citrus sinensis, Jatropha curcas, Glycine max, Gossypium ssp., Populus trichocarpa, Mus musculus, Rattus norvegicus or Homo sapiens.

As used herein, “nucleotide” can thus generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example and not limitation, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited to fluorescein, 5-carboxyfluorescein (FAM), 2′7′-5 dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-l-sulfonic acid (EDANS).

As used herein, “non-native” or “non-naturally occurring” or “artificial” can refer to a nucleic acid or polypeptide sequence, or any other biomolecule like biotin or fluorescein that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence.

The term “phytotoxic” or “phytotoxicity” as used herein in the context of plant cells, tissues, organs or plants, refers to a cytotoxic effect or cytotoxicity in general for a plant, or any plant cell. The term thus implies a toxic effect by a compound or trigger on a plant inhibiting, damaging or even killing a plant cell, tissue, organ or whole plant. Such damage may be caused by a wide variety of compounds, including herbicides, pesticides, trace metals, toxic effectors induced by a pathogen, salinity phytotoxins or allelochemicals. Additionally, the term also refers to plant phytohormones, for example, but not restricted to hormones for the regulation of plant immune responses, like ethylene, jasmonic acid, and salicylic acid, or plant hormones, such as auxins, abscisic acid (ABA), cytokinins, gibberellins, and brassinosteroids, that regulate plant development and growth.

The term “plant” as used herein refers to a whole plant organism, a plant organ, differentiated and undifferentiated plant tissues, plant cells, seeds, and derivatives and progeny thereof. “Plant cells” include without limitation, for example, cells from seeds, from mature and immature embryos, meristematic tissues, seedlings, callus tissues in different differentiation states, leaves, flowers, roots, shoots, gametophytes, sporophytes, pollen and microspores, protoplasts, macroalgae and microalgae. The different plant cells can either be haploid, diploid or multiploid. The term “plant organ” refers to plant tissue or a group of tissues that constitute a morphologically and functionally distinct part of a plant.

A “plant material” as used herein refers to any material which can be obtained from a plant during any developmental stage. The plant material can be obtained either in planta or from an in vitro culture of the plant or a plant tissue or organ thereof The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

A “plasmid” refers to a circular autonomously replicating extrachromosomal element in the form of a double-stranded nucleic acid sequence. In the field of genetic engineering these plasmids are routinely subjected to targeted modifications by inserting, for example, genes encoding a resistance against an antibiotic or an herbicide, a gene encoding a target nucleic acid sequence, a localization sequence, a regulatory sequence, a tag sequence, a marker gene, including an antibiotic marker or a fluorescent marker, and the like. The structural components of the original plasmid, like the origin of replication, are maintained. According to certain embodiments of the present invention, the localization sequence can comprise a nuclear localization sequence, a plastid localization sequence, preferably a mitochondrion localization sequence or a chloroplast localization sequence. Said localization sequences are available to the skilled person in the field of plant biotechnology. A variety of plasmid vectors for use in different target cells of interest is commercially available and the modification thereof is known to the skilled person in the respective field.

“Polymerase chain reaction” (PCR) is a technique for synthesizing a specific DNA segment. PCR comprises a series of repetitive denaturation, annealing, and extension cycles. Typically, a double-stranded DNA is heat denatured, and two primers complementary to the 3′ boundaries of the target segment are annealed to the DNA at low temperature, and then extended at an intermediate temperature. One set of these three consecutive steps is referred to as a “cycle”.

“Progeny” comprises any subsequent generation of a plant, plant cell or plant tissue.

The term “regulatory sequence” as used herein refers to a nucleic acid or an amino acid sequence, which can direct the transcription and/or translation and/or modification of a nucleic acid sequence of interest.

The terms “protein”, “amino acid” or “polypeptide” are used interchangeably herein and refer to an amino acid sequence having a catalytic enzymatic function or a structural or a functional effect. The term “amino acid” or “amino acid sequence” or “amino acid molecule” comprises any natural or chemically synthesized protein, peptide, polypeptide and enzyme or a modified protein, peptide, polypeptide and enzyme, wherein the term “modified” comprises any chemical or enzymatic modification of the protein, peptide, polypeptide and enzyme, including truncations of a wild-type sequence to a shorter, yet still active portion.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S J. Higgins eds. (1985); Transcription and Translation (B. D. Hames & S. J. Higgins, eds. (1984); Animal Cell Culture (R I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

As used herein, “selectable phenotypes”, or “phenotypically selectable” or “phenotypically screenable” defines alterations in the cell or organism's performance or visual characteristics with respect to growth, metabolism, sensitivity to a phytotoxic (e.g., herbicide) or other compound, or consumption of nutrients. A “selectable phenotype” also includes the visible or invisible appearance as observed by eye or using special equipment. A phenotypically selectable trait is thus encoded by at least one genomic region and results in a phenotype which can be screened visually microscopically, or by any means of molecular or analytical biology.

Whenever the present disclosure relates to the percentage of the homology or identity of nucleic acid or amino acid sequences these values define those as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see www.ebi.ac.uk/Tools/psa/ and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

The term “strand break” when made in reference to a double-stranded nucleic acid sequence, e.g., a genomic sequence as DNA target sequence, includes a single-strand break and/or a double-strand break. A single-strand break (a nick) refers to an interruption in one of the two strands of the double-stranded nucleic acid sequence. This is in contrast to a double-strand break which refers to an interruption in both strands of the double-stranded nucleic acid sequence. Strand breaks according to the present disclosure may be introduced into a double-stranded nucleic acid sequence by enzymatic incision at a nucleic acid base position of interest using a suitable endonuclease, including a CRISPR endonuclease or a variant thereof, where the variant can be a mutated or truncated version of the wild-type protein or endonuclease, which still can exert the enzymatic function of the wild-type protein.

The term “target region”, “target site”, “target structure”, “target construct”, “target nucleic acid” or “target cell/tissue/organism”, or “DNA target region” as used herein refers to a target which can be any genomic or epigenomic region within any compartment of a target cell.

The term “targeted or “site-specific” or “site-directed” as used herein refers to an action of molecular biology which uses information on the sequence of a genomic region of interest to be modified, and which further relies on information of the mechanism of action of molecular tools, e.g., nucleases, including CRISPR nucleases and variants thereof, TALENs, ZFNs, meganucleases or recombinases, DNA-modifying enzymes, including base modifying enzymes like cytidine deaminase enzymes, histone modifying enzymes and the like, DNA-binding proteins, cr/tracr RNAs, guide RNAs and the like, which allow the in silico prediction of at least one modification to be effected within a genomic target region of interest. Therefore, the relevant molecular tools can be designed and constructed ex vivo or in silico.

The terms “transgene” or “transgenic” as used herein refer to at least one nucleic acid sequence that is taken from the genome of one organism, or produced synthetically, and which is then introduced into host a cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell.

The term “transient introduction” as used herein thus refers to the transient introduction of at least one nucleic acid sequence according to the present disclosure, preferably incorporated into a delivery vector or into a recombinant construct, with or without the help of a delivery vector, into a target structure, for example, a plant cell, wherein the at least one nucleic acid sequence is introduced under suitable reaction conditions so that no integration of the at least one nucleic acid sequence into the endogenous nucleic acid material of a target structure, the genome as a whole, occurs, so that the at least one nucleic acid sequence will not be integrated into the endogenous DNA of the target cell. As a consequence, in the case of transient introduction, the introduced genetic construct will not be inherited to a progeny of the target structure, for example a prokaryotic, an animal or a plant cell. The at least one nucleic acid sequence or the products resulting from transcription or translation thereof are only present temporarily, i.e., in a transient way, in constitutive or inducible form, and thus can only be active in the target cell for exerting their effect for a limited time. Therefore, the at least one nucleic acid sequence introduced via transient introduction will not be heritable to the progeny of a cell. The effect which a nucleic acid sequence introduced in a transient way can, however, potentially be inherited to the progeny of the target cell.

A “variant” of any site-specific effector or base editor disclosed herein represents a molecule comprising at least one mutation, deletion or insertion in comparison to the respective wild-type enzyme to alter the activity of the wild-type enzyme as naturally occurring. A “variant” can, as non-limiting example, be a catalytically inactive Cas9 (dCas9), or a site-specific nuclease, which has been modified to function as nickase.

### DETAILED DESCRIPTION

The present invention provides methods for targeted editing in a plant cell, tissue, organ or material, which methods specifically combined and use a parallel introduction strategy. The methods provided herein thus rely on the parallel introduction of a phenotypically selectable trait at a first genomic target site, wherein this phenotypically selectable trait as such allows for an easy screening and does not comprise the introduction of a transgenic marker sequence or marker cassette. In addition, the introduction of a targeted modification at a first genomic target site to obtain a selectable phenotype does not rely on the provision of an exogenous polynucleotide template, nor does it rely on the introduction of a double-stand (ds) break at the target site, which steps are usually needed for a variety of genome editing approaches using site-specific nucleases (SSNs) introducing a double-strand break at a genomic target site, which is often cured by providing a repair template for homologous repair (HR) as exogenous nucleic acid material.

There are thus provided methods with specific relevance for plant breeding strategies, where traits of agronomic interest have to be combined within a plant of interest, which usually requires iterative and usually time-consuming steps of selection. Furthermore, the specific method steps provided herein parallelize transgenic marker-free selection and targeted editing at different genomic target sites which results in conferring a selectable or other phenotype to a plant or plant cell. This in turn enables the isolation of such modified plant material without a selection marker cassette, whereas this phenotypical selection can dramatically reduce the costs for screening for a second targeted modification of interest, which is usually not phenotypically screenable as such. Due to this synergistic interplay of the simultaneous introduction of two targeted modifications, one modification guaranteeing transgenic marker-free selection, and the second modification allowing the introduction of a highly site-specific and predictable edit into a genomic target site of interest, the methods of the present invention allow precision breeding strategies comprising significantly reduced selection efforts for identifying a genotype of interest, which in turn helps to reduce time and costs necessary to identify relevant modifications within a plant cell or germplasm of interest.

In a first aspect, there is provided a method for isolating at least one modified plant cell or at least one modified plant tissue, organ, or whole plant comprising the at least one modified plant cell, without stably integrating a transgenic selectable marker sequence, the method comprising: (a) introducing at least one first targeted base modification into a first plant genomic target site of at least one plant cell to be modified, wherein the at least one targeted base modification causes expression of at least one phenotypically selectable trait; (b) introducing at least one second targeted modification into a second plant genomic target site of the at least one plant cell to be modified, wherein the at least one second targeted modification is introduced using at least one of a site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted base modification at the first plant genomic target site, and optionally by further selecting (ii) for the at least one second targeted modification in the second plant genomic target site.

No stable integration of a transgenic exogenous sequence to be used as selectable marker is necessary according to the methods of the present invention. Instead, a phenotypically selectable trait or phenotype is made at a first plant genomic target site. This has the advantage of providing a selectable edit, which does not rely on the integration of an exogenous nucleic acid construct to be used as marker during selection.

A “phenotypically selectable trait” as used herein refers to a trait encoded by at least one gene causing a visible or otherwise selectable phenotype after expression of the relevant genomic trait. Selection for said trait can be accomplished visually, or by using a selection agent, compound or trigger to be applied to a plant cell, tissue, organ, material or whole plant.

The first and the second plant genomic target site can be the same, or different genomic loci. Preferably, the first and the second plant genomic target site reside within different genomic loci, which genomic loci can be located on the same, or on different chromosomes.

According to the methods of the present invention, a parallel introduction strategy of a first and a second targeted modification is made, wherein this parallelization of the different targeted modifications introduced at a first and at a second plant genomic target site significantly improves the later screening steps. Usually, the second modification will have no opportunity for selection because the phenotype it confers will not be expressed or relevant in the process of generating the plants. So the purpose underlying the methods of the present invention is to use the first modification causing a phenotypically selectable phenotype as a tool to enable selection. Compared to traditional methods, the methods disclosed herein have the advantage of not incorporating a transgenic marker gene. Compared to not using a selectable phenotype with a selection agent, it has the advantage of increased efficiency by eliminating all or most untreated cells, which would otherwise comprise the majority the cells producing plants. By eliminating untreated cells, the number of plants that have to be produced is greatly reduced, and the number of plants that have to be molecularly screened for the second targeted modification is greatly reduced, which in turn increases the efficiency of the disclosed methods for plant breeding.

Preferably, the methods according to the various aspects of the present invention rely on the simultaneous or subsequent introduction of the at least one first targeted base modification, codon deletion or frameshift or deletion modification into the same at least one plant cell to be modified also receiving the at least one second targeted modification into a second plant genomic target site of interest. The modifications at the first and the second target site are thus preferably introduced at the same time into the same cell, i.e., in a simultaneous way, i.e., in parallel. The subsequent introduction in this sense thus refers to the fact that the different tools introduced comprising at least one base editing complex and/or at least one site-specific effector might act shortly before each other. Still, the term subsequently in this context implies that the parallel and simultaneous introduction of the tools of interest within the same cell. This in turn has the effect of improving screening possibilities due to the fact that coupling of the introduction processes for the molecular tools mediating the at least one first and second targeted modification the modifications are not completely independent of each other. Cells to be modified having one modification are thus much more likely to also have the second targeted modification. Compared to selecting cells at random, particularly for the second modification usually not having a clear phenotype from the whole population of treated and untreated cells, the methods of the present invention provide selection advantages. Selection is thus significantly improved, as the delivery of the respective tools in a functional way, which usually represents a bottleneck during genome editing, is synchronized and done simultaneously. Due to the possibility of selecting for the first modification in a targeted way, a limited number of screening efforts for the at least one targeted modification of the second plant genomic target site thus has to be done, as cells which did not receive any tool or complex according to the present invention in a functional way at all will not have received a modification leading to a phenotypically selectable trait at the first plant genomic target site. As the chance that said plant cells received the second site-specific effector complex according to the present invention added to the cells in parallel is low, no time-consuming screening will have to be done for the second targeted modification, in case that the screening for the first targeted modification is negative.

The methods according to the present invention thus make it possible for cells to select for cells that did, or did not receive the at least one first modification by selecting for the phenotypically selectable trait targeted with the first targeted modification by suitable reagents, or by visual screening. Therefore, this screening eliminates cells not comprising the at least one first modification, or the screening allows the visual inspection and separation of cells into modified cells having received, or not having received the first targeted modification. Of the cells having successfully received the first targeted modification, a reasonable number can be expected to also have the at least one second targeted modification as well due to the parallel introduction and delivery approach according to the present invention. “Reasonable” in this context implies any improvement, i.e., a decrease, of the number of cells to be screened for the presence of the at least one second targeted modification by selecting for the at least one phenotypically selectable trait caused by the at least one first targeted base modification. The actual frequency of the presence of the at least one second targeted modification is usually hard to predict as it will be variable depending on several factors. This makes screening for any modification introduced via genome engineering cumbersome to screen for using common molecular techniques, e.g., relying on PCR: According to the methods of the present invention, the frequency of cells having received both, the first and the second targeted modification can be in the range of between 2:1 and 1,000:1 plant cells or plants having the first modification compared to those having the first and second modifications. Therefore, there is an intrinsic advantage during any screening or selection step, as the total number of cells which has to be screened for the second modification will be reduced. Particularly, those cells, where the delivery of the tools for introducing the first and the second targeted modification failed will likely not have received any molecular tool(s) and thus neither the first nor the second targeted modification can be present. No first phenotypically selectable trait will thus be apparent, i.e., selectable. Under selective pressure, or after visual selection, the corresponding plant cells, tissues, organs or whole plants “negative” for the phenotypically selectable trait will not have to be subjected to subsequent screenings for the second targeted modification, as the likelihood that the second modification was introduced, when the first modification is not present, is low due to the parallel introduction of the respective tools.

If desired, the first modification can be removed by crossing the derived plant and genetically segregating it away from the second modification.

The methods disclosed herein can thus be used for enriching recovery of plants with the targeted modification at a second gene of interest by eliminating or removing the cells that did not receive the editing reagents or did not undergo the targeted modification as screened for the at least one first targeted modification of interest.

A targeted base modification according to the various embodiments of the present invention refers to a to genome editing that enables the direct, irreversible conversion of one target DNA base into another in a programmable manner, without requiring dsDNA backbone cleavage or a donor template (cf. Komor et al., Nature, Vol. 533, 2016).

In one embodiment, the methods according to the first aspect of the present invention additionally comprise, within step (b), introducing a repair template to make a targeted sequence conversion or replacement at the at least second plant genomic target site. A repair template (RT) represents a single-stranded or double-stranded nucleic acid sequence, which can be provided during any genome editing causing a double-strand or single-strand DNA break to assist the targeted repair of said DNA break by providing a RT as template of known sequence assisting homology-directed repair. The size of the at least one repair template nucleic acid sequence according to the present invention as part can vary. It can be in the range from about 20 bp to about 5,000 bp or even 8,000 bp depending on the DNA target sequence to be modified in a site-directed way. The RT can be provided as individual physical entity, or as part of a complex according to the present invention. The use of a RT might be favorable for certain applications to avoid undesired insertions or deletions due to a cellular NHEJ repair mechanism.

In one embodiment according to the various aspects of the present invention, the methods provided herein comprise a further step of (d) crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

The further plant or plant material of interest can be any plant material comprising genomic material of interest, wherein this material, comprising, for example, an elite event or any trait of interest, is intended, e.g., for subsequent rounds of breeding to create a genotype and thus a plant of interest. The genotype of interest is thus the result of preceding breeding steps combining traits from different plants of interest.

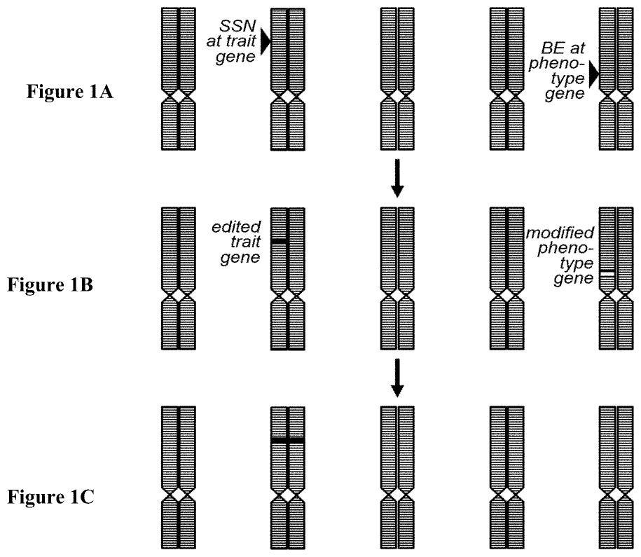

In one embodiment according to all aspects of the present invention, the final genotype of interest does not comprise the at least one first targeted modification, i.e. the at least one phenotypically selectable trait. As illustrated in FIG. 1, the methods of the present invention are particularly suitable to remove the first targeted modification causing a phenotypically selectable trait by crossing the derived plant and genetically segregating it away from the second targeted modification (cf. FIG. 1C), if desired for certain applications. In another embodiment, the first targeted modification encoding a phenotypically selectable trait of interest can be kept in the genotype of interest in case that the phenotypically selectable trait as such has a value for the resulting genotype of interest and the corresponding plant or plant material.

In one embodiment according to the first aspect of the present invention, wherein the at least one site-specific effector is temporarily or permanently linked to at least one base editing complex, wherein the base editing complex mediates the at least one first targeted base modification of step (a). The at least one site-specific effector can thus be non-covalently (temporarily) or covalently (permanently) be attached to at least one base editing complex. Any component of the at least one base editing complex can be temporarily or permanently linked to the at least one site-specific effector. The terms “temporarily” and “permanently” are thus to be construed broadly and comprise both covalent and/or non-covalent bonds or attachments to achieve physical proximity of the at least one site-specific effector and the least one base editing complex. The linkage of at least on component of the at least one base editing complex and the at least one site-specific effector, or also the any other component, for example a gRNA or a RT associated with the at least one site-specific effector, might be of interest in case the at least one first and the at least one second genomic target site are in close proximity within a genome of interest.

In one embodiment according to the various aspects of the present invention, the at least one site-specific effector is selected from at least one of a nuclease, comprising a CRISPR nuclease, including Cas or Cpf1 nucleases, a TALEN, a ZFN, a meganuclease, an Argonaute nuclease, a restriction endonuclease, including FokI or a variant thereof, a recombinase, or two site-specific nicking endonucleases, or a base editor, or any variant or catalytically active fragment of the aforementioned effectors.

A “site-specific effector” as used herein can thus be defined as any nuclease, nickase, recombinase, or base editor, having the capacity to introduce a single- or double-strand cleavage into a genomic target site, or having the capacity to introduce a targeted modification, including a point mutation, an insertion, or a deletion, into a genomic target site of interest. The at least one “site-specific effector” can act on its own, or in combination with other molecules as part of a molecular complex. The “site-specific effector” can be present as fusion molecule, or as individual molecules associating by or being associated by at least one of a covalent or non-covalent interaction so that the components of the site-specific effector complex are brought into close physical proximity.

A “base editor” as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Preferably, the at least one base editor according to the present invention temporarily or permanently linked to at least one site-specific effector, or optionally to a component of at least one site-specific effector complex. The linkage can be covalent and/or non-covalent.

Any base editor or site-specific effector, or a catalytically active fragment thereof, or any component of a base editor complex or of a site-specific effector complex as disclosed herein can be introduced into a cell as a nucleic acid fragment, the nucleic acid fragment representing or encoding a DNA, RNA or protein effector, or it can be introduced as DNA, RNA and/or protein, or any combination thereof.

A key toolset that eliminates the requirement for making selectable modifications with an endonuclease, a DSB, and a repair template is the use of base editors or targeted mutagenesis domains. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalysed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. We reasoned that a subset of this stretch of ssDNA in the R-loop might serve as an efficient substrate for a dCas9-tethered cytidine deaminase to effect direct, programmable conversion of C to U in DNA (Komor et al., supra).

Any base editing complex according to the present invention can thus comprise at least one cytidine deaminase, or a catalytically active fragment thereof The at least one base editing complex can comprise the cytidine deaminase, or a domain thereof in the form of a catalytically active fragment, as base editor.

In another embodiment, the at least one first targeted base modification is a conversion of any nucleotide C, A, T, or G, to any other nucleotide. Any one of a C, A, T or G nucleotide can be exchanged in a site-directed way as mediated by a base editor, or a catalytically active fragment thereof, to another nucleotide. The at least one base editing complex can thus comprise any base editor, or a base editor domain or catalytically active fragment thereof, which can convert a nucleotide of interest into any other nucleotide of interest in a targeted way.

The present invention provides methods combining the knowledge of the base editor tools as such and uses this technology in a combined method for achieving a phenotypically selectable phenotype of interest to avoid the need of a transgenic marker, as the base edit can artificially create an endogenous marker having a phenotypical output being selectable. To this end, a base editor is combined with a modified site-specific effector that retains the ability to recognize and bind a genomic target region, optionally guided by a gRNA for CRISPR-based nucleases, to mediate the conversion of C to U, or G to A, to introduce a site directed mutagenesis. In turn, targeted mutations can be effected which result in a phenotype of interest. This paves the way for targeted breeding strategies, particularly as the methods disclosed herein additionally combine the use of at least one base editor or base editing complex to introduce a targeted base modification into a first plant genomic target site of at least one plant cell to be modified with a second modification mediated by at least one site-specific effector in a parallel way. This approach allows marker-free selection and screening for a modification or a genotype of interest in a synergistic way, without the need to introduce a DSB or a RT for the at least one first modification according to the various aspects of the present invention, i.e., for a targeted base modification, a targeted codon deletion, or a targeted frameshift or deletion modification.

The addition of a uracil DNA glycosylase (UGI) domain further increased the base-editing efficiency. A nuclear localization signal (NLS), or any other organelle targeting signal, can be further required to ensure proper targeting of the complex.

In one embodiment according to all aspects of the present invention, the at least one site-specific effector is a CRISPR-based nuclease, wherein the CRISPR-based nuclease comprises a site-specific DNA binding domain directing the at least one base editing complex, wherein the at least one CRISPR-based nuclease, or the nucleic acid sequence encoding the same, is selected from the group comprising (a) Cas9, including SpCas9, SaCas9, SaKKH-Cas9, VQR-Cas9, St1Cas9, (b) Cpf1, including AsCpf1, LbCpf1, FnCpf1, (c) CasX, or (d) CasY, or any variant or derivative of the aforementioned CRISPR-based nucleases, preferably wherein the at least one CRISPR-based nuclease comprises a mutation in comparison to the respective wild-type sequence so that the resulting CRISPR-based nuclease is converted to a single-strand specific DNA nickase, or to a DNA binding effector lacking all DNA cleavage ability.

A “CRISPR-based nuclease”, as used herein, is any nuclease which has been identified in a naturally occurring CRISPR system, which has subsequently been isolated from its natural context, and which preferably has been modified or combined into a recombinant construct of interest to be suitable as tool for targeted genome engineering. Any CRISPR-based nuclease can be used and optionally reprogrammed or additionally mutated to be suitable for the various embodiments according to the present invention as long as the original wild-type CRISPR-based nuclease provides for DNA recognition, i.e., binding properties. Said DNA recognition can be PAM dependent. CRISPR nucleases having optimized and engineered PAM recognition patterns can be used and created for a specific application. The expansion of the PAM recognition code can be suitable to target the site-specific effector complexes to a target site of interest, independent of the original PAM specificity of the wild-type CRISPR-based nuclease. Cpf1 variants can comprise at least one of a S542R, K548V, N552R, or K607R mutation, preferably mutation S542R/K607R or S542R/K548V/N552R in AsCpf1 from Acidaminococcus (cf. SEQ ID NO:24).

Furthermore, modified Cas variant, e.g., Cas9 variants, can be used according to the methods of the present invention as part of a base editing complex, e.g. BE3, VQR-BE3, EQR-BE3, VRER-BE3, SaBE3, SaKKH-BE3 (see Kim et al., Nat. Biotech., 2017, doi:10.1038/nbt.3803). Therefore, according to the present invention, artificially modified CRISPR nucleases are envisaged, which might indeed not be any “nucleases” in the sense of double-strand cleaving enzymes, but which are nickases or nuclease-dead variants, which still have inherent DNA recognition and thus binding ability. Exemplary Cas- or Cpf1-based constructs suitable for the purpose of the present invention are disclosed in SEQ ID NOs:17 to 19. An AsCpf1 wild-type sequence is disclosed in SEQ ID NO:24. Other suitable Cpf1-based effectors for use in the methods of the present invention are derived from Lachnospiraceae bacterium (LbCpf1, e.g., NCBI Reference Sequence: WP_051666128.1), or from Francisella tularensis (FnCpf1, e.g., UniProtKB/Swiss-Prot: A0Q7Q2.1). Variants of Cpf1 are known (cf. Gao et al., BioRxiv, dx.doi.org/10.1101/091611). Variants of AsCpf1 with the mutations S542R/K607R and S542R/K548V/N552R that can cleave target sites with TYCV/CCCC and TATV PAMs, respectively, with enhanced activities in vitro and in vivo are thus envisaged as site-specific effectors according to the present invention. Genome-wide assessment of off-target activity indicated that these variants retain a high level of DNA targeting specificity, which can be further improved by introducing mutations in non-PAM-interacting domains. Together, these variants increase the targeting range of AsCpf1 to one cleavage site for every ˜8.7 bp in non-repetitive regions of the human genome, providing a useful addition to the CRISPR/Cas genome engineering toolbox (see Gao et al., supra).

In one embodiment according to the first aspect of the present invention, the at least one first targeted base modification is made by at least one base editing complex comprising at least one base editor as component. The base editing complex according to the present invention comprises the base editor as well as further optional components.

In one embodiment, the base editing complex contains an APOBEC1 component, preferably a rat APOBEC1. In another embodiment, the base editing complex can comprise any cytidine/cytosine deaminase enzyme as base editor, for example a human AID, e.g., UniProtKB/Swiss-Prot: Q9GZX7.1, a human APOBEC3G, e.g., GenBank: CAK54752.1, or a lamprey CDA1, e.g. GenBank: ABO15150.1, but any enzyme or catalytically active fragment thereof is envisaged within the scope of the present invention. An exemplary APOBEC component suitable for use in the methods of the present invention is represented by SEQ ID NO:20. Furthermore, a modified base editor can be used according to the methods of the present invention, preferably a base editor having a narrow editing width of below 6 nt, below 5 nt, below 4 nt, below 3 nt, or event 2 nt or 1 nt. The narrower the editing window, the more precise an edit can be introduced at a genomic target site of interest.

In one embodiment, the base editing complex contains an UGI (uracil DNA glycosylase inhibitor) component. In certain embodiments, a UGI derived from Bacillus subtilis can be used, or any other domain inhibiting UDG activity to repress the activity of endogenous base-excision repair (BER) active in certain cells. An exemplary UGI component suitable for use in the methods of the present invention is represented by SEQ ID NO:21.

In yet a further embodiment, the base editing complex contains a XTEN component i.e., a specific linker to provide optimum deamination activity of the at least one base editor linked to the at least one site-specific effector. Other linkers having a length of at least 2 nucleotide (nt) between the base editor and the site-specific effector can be used, which do not influence the binding activity as conferred by the site-specific effector and/or the base editing activity of the base editor. A suitable XTEN linker sequence is provided with SEQ ID NO:1 (position 688 to 735), SEQ ID NO:2 (position 706 to 753), SEQ ID NO:14 (position 706 to 753), or SEQ ID NO:15 (position 706 to 753). There is a variety of further linkers known to the skilled person as well as literature on linker design. Both, rigid as well as flexible linkers can thus be used according to the various methods of the present invention.

Exemplary fusion constructs according to the present invention are provided with SEQ ID NOs:1, 2, 14, 15, or 16.

In one embodiment, the at least one base editing complex comprises more than one component, and wherein the at least two components are physically linked. A physical linkage can comprise a covalent linkage, e.g., by fusing DNA fragments to each other to create a fusion protein after expression, or by chemically crosslinking different components of a complex according to the present disclosure to each other. A physical linkage can additionally comprise a non-covalent interaction. Non-covalent interactions or attachments thus comprise electrostatic interactions, van der Waals forces, TT-effects and hydrophobic effects. Of special importance in the context of nucleic acid molecules are hydrogen bonds as electrostatic interaction. A hydrogen bond (H-bond) is a specific type of dipole-dipole interaction that involves the interaction between a partially positive hydrogen atom and a highly electronegative, partially negative oxygen, nitrogen, sulfur, or fluorine atom not covalently bound to said hydrogen atom.

In a further embodiment, the base editing complex contains a PmCDA1 (activation-induced cytidine deaminase (AID) ortholog PmCDA1 from sea lamprey, see Nishida et al. (Science 2016, vol. 353, issue 6305, aaf8729)) component as base editor. An exemplary PmCDA1 for use according to the methods of the present invention is provided with SEQ ID NO:22.

CRISPR-based nucleases act via recognition of a protospacer-adjacent motif (PAM) present within a genomic target region of interest to be modified. To further increase the scope and precision of base editing using modified CRISPR-based nucleases, the introduction of different PAM specificities to expand the number of sites that can be targeted is thus of great interest (Kim et al., Nat. Biotech., 2017, doi:10.1038/nbt.3808). As it is known to the skilled person, wild-type CRISPR nucleases have intrinsic PAM specificities varying from nuclease to nuclease. According to the present invention, CRISPR-based nucleases are this envisaged, which have an altered PAM specificity and thus a modified targeting range, for example, SpCas9 mutants that accept NGA (VQR-Cas9), NGAG (EQR-Cas9), or NGCG (VRER-Cas9) PAM sequences, as well as an engineered SaCas9 variant containing three mutations (SaKKH-Cas9) that relax the variant's PAM requirement to NNNRRT (Kleinstiver et al., Nat. Biotechnol. 33, 1293-1298 (2015)). Exemplary PAM sequences according to the present invention suitable for different CRISPR-based nucleases are represented by SEQ ID NOs: 3 to 13 and 23.

In one embodiment, the at least one base editing complex comprises more than one component, wherein the at least two components are provided as individual components. This approach can be suitable for certain transformation or transfection strategies.

In certain embodiments according to the methods of the present invention, at least one component of any complex according to the present invention can comprise a part or portion, which can specifically interact or associate with a cognate binding partner within a cell of interest so that a complex will form within the cell, or the complex can be formed ex vivo before transformation or transfection. The binding pairs can associate via a docking domain or association domain, or the nucleic acid sequence encoding the same, which is selected from at least one of biotin, an aptamer, a DNA, RNA or protein dye, comprising fluorophores, comprising fluorescein, or a variant thereof, maleimides, or Tetraxolium (XTT), a guide nucleic acid sequence specifically configured to interact with a at least one repair template nucleic acid sequence, a streptavidin, or a variant thereof, preferably a monomeric steptavidin, an avidin, or a variant thereof, an affinity tag, preferably a streptavidin-tag, an antibody, a single-chain variable fragment (scFv), an antigen specific for a given antibody or scFv, a single-domain antibody (nanobody), an anticalin, an Agrobacterium VirD2 protein or a domain thereof, a Picornavirus VPg, a topoisomerase or a domain thereof, a PhiX174 phage A protein, a PhiX A* protein, a VirE2 protein or a domain thereof, or digoxigenin. Other suitable binding pairs are known to the skilled person. Most preferably, the cognate binding partners have a high affinity constant or bonding affinity and thus a low dissociation constant (K_{d}) for each other under physiological conditions, i.e. a K_{d} value in the low μM, or preferably nM range, and preferably below to assist in complex formation of the at least one base editing complex, or the at least one site-specific effector complex according to the present invention.

In one embodiment according to all aspects of the methods of the present invention, at least one component of the at least one base editing complex, and/or at least one component of the at least one site-specific effector complex comprises at least one organelle localization signal to target the at least one base editing complex to a subcellular organelle. In one embodiment, the at least one organelle localization signal is a nuclear localization signal (NLS). In a further embodiment, the at least one organelle localization signal is a chloroplast transit peptide. In yet a further embodiment, the at least one organelle localization signal is a mitochondria transit peptide. One or more localization signal(s) can be present being associated with at least one component of the base editing, or the site-specific effector complex.

On one embodiment according to the various aspects of the present invention, the first plant genomic target site of the at least one plant cell is a genomic target site encoding at least one phenotypically selectable trait, wherein the at least one phenotypically selectable trait is a resistance/tolerance trait or a growth advantage trait, and wherein the at least one first targeted base modification at the first plant genomic target site of the at least one plant cell confers resistance/tolerance or a growth advantage towards a compound or trigger to be added to the at least one modified plant cell, tissue or plant, or a progeny thereof.

A “growth advantage” as used herein refers to any physiologically or metabolically favourable property during all stages of plant development and reproduction, for example, favouring the resistance to biotic and abiotic stress, or influencing plant growth and development, e.g. under stress conditions like drought, salinity, and the like.

A “compound” or “trigger” according to the present invention can thus be a herbicide, for example being selected from cell metabolism inhibitors, for example: EPSPS inhibition (glycines, e.g., glyphosate); ALS/AHAS (branched amino acid production) inhibition (for example, imidazolines, sulfonylurea); lipid synthesis inhibition/ACCases (aryloxyphenoxypropionate (FDPs), cyclohexanedione (DIMs), phenylpyrazolin (DENs); inhibitors of glutamine synthetase (glufosinate/phosphinotricin), growth/cell division inhibitors, for example, disruptors of plant cell growth (phenoxycarboxylic acids, e.g., 2,4-D), synthetic auxins (benzoic acid e.g., dicamba), auxin transport inhibition (phtalamates); and interference with light processes, for example: bleachers/inhibitors of HPPDs (pyrazoles and isoxazole); inhibitors of photosystem II (PS II inhibitors) (triazines, triazinones, pyridazones, C3: ioxynil and bromoxynil and many others); inhibitors of protoporphyrinogen oxidase (PPO/PPX) (e.g., diphenylethers and N-phenylphtalimides).

Furthermore, a “compound” or “trigger” according to the present invention can be a plant growth factor or any other substance, endogenously produced by a plant, or exogenously applied, which influences plant metabolism.

For all embodiments of the methods disclosed herein, the compound or trigger can be exogenously applied to allow selection for a trait of interest, the phenotypically selectable trait encoded by the at least one plant cell, tissue, organ, material or whole plant, an modified in a targeted way according to the various methods of all aspects of the present invention. The provision of a specific interaction pair in the form of the modification of a phenotypically selectable trait and the provision of a corresponding compound or trigger during subsequent selection and crossing steps, therefore, can improve any breeding effort.

In one embodiment according to the various aspects of the present invention, the at least one phenotypically selectable trait of interest is or is encoded by at least one endogenous gene, or wherein the at least one phenotypic trait of interest is or is encoded by at least one transgene, wherein the at least one endogenous gene or the at least one transgene encode(s) at least one phenotypic trait selected from the group consisting of resistance/tolerance to a phytotoxin, preferably a herbicide, inhibiting, damaging or killing cells lacking the at least one modification at the at least one phenotypic trait of interest, or wherein the at least one phenotypic trait is selected from the group consisting of boosters of cell division, growth rate, embryogenesis, or another phenotypically selectable property that provides an advantage to a modified cell, tissue, organ, or plant compared to an unmodified cell, tissue, organ, or plant.

In a further embodiment according to the various aspects of the present invention, the at least one first plant genomic target site is at least one endogenous gene or a transgene encoding at least one phenotypically selectable trait selected from the group consisting of herbicide resistance/tolerance, wherein the herbicide resistance/tolerance is selected from the group consisting of resistance/tolerance to EPSPS-inhibitors, including glyphosate, resistance/tolerance to glutamine synthesis inhibitors, including glufosinate, resistance/tolerance to ALS- or AHAS-inhibitors, including imidazoline or sulfonylurea, resistance/tolerance to ACCase inhibitors, including aryloxyphenoxypropionate (FOP), resistance/tolerance to carotenoid biosynthesis inhibitors, including inhibitors of carotenoid biosynthesis at the phytoene desaturase step, inhibitors of 4-hydroxyphenyl-pyruvate-dioxygenase (HPPD), or inhibitors of other carotenoid biosynthesis targets, resistance/tolerance to cellulose inhibitors, resistance/tolerance to lipid synthesis inhibitors, resistance/tolerance to long-chain fatty acid inhibitors, resistance/tolerance to microtubule assembly inhibitors, resistance/tolerance to photosystem I electron diverters, resistance/tolerance to photosystem II inhibitors, including carbamate, triazines and triazinones, resistance/tolerance to PPO-inhibitors and resistance/tolerance to synthetic auxins, including dicamba (2,4-D, i.e., 2,4-dichlorophenoxyacetic acid).

In a further embodiment according to the various aspects of the present invention the at least one endogenous gene or the at least one transgene encode(s) at least one phenotypic trait selected from the group consisting of resistance/tolerance to biotic stress, including pathogen resistance/tolerance, wherein the pathogen is selected from a virus, a bacterial, fungal, or an animal pathogen, resistance/tolerance to abiotic stress, including chilling resistance/tolerance, drought stress resistance/tolerance, osmotic resistance/tolerance, heat stress resistance/tolerance, cold stress resistance/tolerance, oxidative stress resistance/tolerance, heavy metal stress resistance/tolerance, salt stress or waterlogging resistance/tolerance, lodging resistance/tolerance, shattering resistance/tolerance, or wherein the at least one phenotypic trait of interest is selected from the group consisting of the modification of a further agronomic trait of interest, including yield increase, flowering time modification, seed color modification, endosperm composition modification, nutritional content modification, or metabolic engineering of a pathway of interest.

In one embodiment according to the various aspects of the present invention, the at least one phenotypically selectable trait is a phytotoxic resistance/tolerance trait, preferably a herbicide resistance/tolerance trait, and wherein the at least one first targeted base modification at the first plant genomic target site of the at least one plant cell to be modified confers resistance/tolerance for a phytotoxic compound, preferably a herbicide, said compound being an exogenous compound to be added to the at least one modified plant cell, tissue, organ, or whole plant, or a progeny thereof.

Any further phenotypically selectable trait encoded by the genome of a plant cell of interest can be made the target of the at least one first targeted modification according to the various aspects of the present invention provided that at least one gene is known encoding a phenotypically selectable trait of interest, and provided that a corresponding and complementary compound or trigger is available or can be designed to screen for a targeted modification. For visible phenotypes no compound or trigger is necessary for screening purposes, instead, a suitable read-out and determination strategy based on the observation of visually screenable traits has to be at hand.

In one embodiment according to the various aspects, the first plant genomic target site of the at least one plant cell is a gene conferring resistance or tolerance to a herbicide or a phytotoxic compound, wherein the first plant genomic target site comprises at least one nucleic acid conversion resulting in at least one corresponding amino acid conversion, wherein the at least one nucleic acid conversion is made by at least one base editor.

In one embodiment according to the various aspects of the present invention, the first plant genomic target site of the at least one plant cell is ALS. Any ALS sequence is suitable for the purpose of the present invention. An exemplary ALS sequence is represented by SEQ ID NO:25.

In one embodiment according to the various aspects of the present invention, the first plant genomic target site of the at least one plant cell is PPO. Any PPO sequence is suitable for the purpose of the present invention. An exemplary PPO sequence is represented by SEQ ID NO:26.

In one embodiment according to the various aspects of the present invention, the first plant genomic target site of the at least one plant cell is EPSPS. Any EPSPS sequence is suitable for the purpose of the present invention. An exemplary EPSPS sequence is represented by SEQ ID NO:27.

In one embodiment according to the various aspects of the present invention, the first plant genomic target site of the at least one plant cell is EPSPS, ALS, or PPO, or any allelic or plant variant thereof, and wherein the EPSPS, ALS or PPO comprises at least one nucleic acid conversion resulting in at least one corresponding amino acid conversion, wherein the at least one nucleic acid conversion is made by at least one base editor.

One such target encoding a phenotypically selectable trait according to the present invention is the 5-enolpyruvylshikimate-3-phosphate synthase (EPSPS) gene. Several single and double amino acid substitutions have been shown to reduce glyphosate sensitivity of the enzyme (Sammons, R. D. and Gaines, T. A. (2014), Glyphosate resistance: state of knowledge. Pest. Manag. Sci., 70: 1367-1377.)

Another target is the acetolactate synthase (ALS) gene, for which a variety of single amino acid mutations have been linked to tolerance to one or more herbicides from the classes triazolopyrimidines, sulfonylureas, pyrimidinylthiobenzonates, imidazolinones, and sulfonylaminocarbonyltriazolinone. Suitable residue substitutions for the purpose of the present invention include A122, P197, A205, D376, W574, and S653).

Yet another selectable modification would be in the protoporphyrinogen oxidase (PPO) gene of Zea mays and Arabidopsis thaliana. Here, a modification of cysteine at position 215 into Phenylalanine (A215F), leucine (A215L), or lysine (A215K), as well as the alanine at position 220 into valine (A220V), threonine (A220T), or leucine (A220L), as well as the glycine at position 221 into serine (A221S) or leucine (A221L) refers resistance to PPO herbicides such as diphenylethers, N-phenylphthalimides, oxadiazoles, oxazolidinediones, phenylpyrazoles, pyrimidinidiones, thiadiazoles, triazolinones, as well as others (Li, Xianggan et al. “Development of Protoporphyrinogen Oxidase as an Efficient Selection Marker for Agrobacterium Tumefaciens-Mediated Transformation of Maize.” Plant Physiology 133.2 (2003): 736-747. PMC. Web. 15 Mar. 2017). In addition to the above mentioned residue substitutions, a single amino acid deletion of the glycine at positon 178 in N tabacum or its homologue hinders PPO inhibitor binding and provides resistance to the above mentioned inhibitors (Patzoldt, W. L. et al. (2006). “A codon deletion confers resistance to herbicides inhibiting protoporphyrinogen oxidase” PNAS 103(33):12329-12334) and can be used according to the various aspects of the present invention.

Furthermore, the technology presented in the present application allows for the precise amino acid modification and deletion as well as the introduction of stop codons to alter or interrupt the sequence of gene that gives rise to a selectable phenotype. Of 61 codons that encode for amino acids, five amino acids can be converted to a stop codon by at least one cytosine/cytidine to thymine/thymidine conversion on either strand.

A tool for making these modifications is a CRISPR nuclease by itself. CRISPR nucleases that were shown to provide single or multiple base pair deletions include Cas9, Cpf1, CasX, and CasY. Although these are the most convenient options at this point, future development of site-directed nucleases will easily be adaptable to the procedures described in this document.

In one embodiment according to the various aspects of the present invention, the first plant genomic target site of the at least one plant cell is ALS, and a targeted modification occurs at the sequence encoding A122 in comparison to an ALS reference sequence according to SEQ ID NO:25, or a targeted modification occurs at the sequence encoding P197 in comparison to an ALS reference sequence according to SEQ ID NO:25, or a targeted modification occurs at the sequence encoding A205 in comparison to an ALS reference sequence according to SEQ ID NO:25, or a targeted modification occurs at the sequence encoding D376 in comparison to an ALS reference sequence according to SEQ ID NO:25, or a targeted modification occurs at the sequence encoding R377 in comparison to an ALS reference sequence according to SEQ ID NO:25, or a targeted modification occurs at the sequence encoding W574 in comparison to an ALS reference sequence according to SEQ ID NO:25, or a targeted modification occurs at the sequence encoding S653 in comparison to an ALS reference sequence according to SEQ ID NO:25, or a targeted modification occurs at the sequence encoding G654 in comparison to an ALS reference sequence according to SEQ ID NO:25, or any combination of the aforementioned mutations.

In one embodiment according to the various aspects of the present invention, the first plant genomic target site of the at least one plant cell is PPO, and a targeted modification occurs at the sequence encoding C215, A220, G221, N425, or Y426 in comparison to an PPO reference sequence according to SEQ ID NO:26, or any combination of the aforementioned mutations.

In one embodiment according to the various aspects of the present invention, the first plant genomic target site of the at least one plant cell is PPX2L gene product from Amaranthus tuberculatus for the purpose of selection. In one embodiment according to the various aspects of the present invention, the first targeted modification, comprising a targeted base modification, a targeted codon deletion, or a targeted frameshift or deletion modification, occurs at the position comparable to the G210 residue of the PPX2L gene product from Amaranthus tuberculatus according to SEQ ID NO:28.

In one embodiment according to the various aspects of the present invention, the first plant genomic target site of the at least one plant cell is EPSPS, and at least one targeted modification occurs at any one of targeted modifications occurs at the sequence encoding G101, T102, P106, G144, or A192 in comparison to an EPSPS reference sequence according to SEQ ID NO:27, or any combination of the aforementioned mutations. In certain preferred embodiments, targeted modifications occur at the sequence encoding G101 and at G144 in comparison to an EPSPS reference sequence according to SEQ ID NO:27, or targeted modifications occur at the sequence encoding G101 and at A192 in comparison to an EPSPS reference sequence according to SEQ ID NO:27, or targeted modifications occur at the sequence encoding T102 and at P106 in comparison to an EPSPS reference sequence according to SEQ ID NO:27.

The person having ordinary skill in the art, based on the disclosure provided herein, can also define further suitable phytotoxic resistance/tolerance traits and corresponding mutations to create at least one phenotypically selectable trait according to the present invention.

In certain embodiments according to the various aspects of the present invention, the at least one phenotypically selectable trait is a visible phenotype that is useful in identifying or isolating at least one modified plant cell, tissue, organ or whole plant. A “visible” phenotype is any phenotype which can be detected by means of observation with the eyes, either macroscopically or microscopically, so that no screening by means of molecular biology becomes necessary.

In one embodiment according to the various aspects of the present invention, the at least one phenotypically selectable trait is a glossy phenotype, a golden phenotype, a pigmentation phenotype, or a growth advantage phenotype. Several other visible phenotypes are known to the skilled person. Said visible phenotypes will vary depending on the plant or plant cell of interest due to its genetic background.

In a second aspect according to the present invention, there is provided a method for isolating at least one modified plant cell or at least one modified plant tissue, organ, or whole plant comprising the at least one modified plant cell, without stably integrating a transgenic selectable marker sequence, the method comprising: (a) introducing at least one first targeted codon deletion modification into a first plant genomic target site of at least one plant cell to be modified using at least one first site-specific effector, comprising a nuclease, a recombinase, or a DNA modification reagent, wherein the at least one targeted codon deletion modification causes expression of at least one phenotypically selectable trait; (b) introducing at least one second targeted modification into a second plant genomic target site of the at least one plant cell to be modified, wherein the at least one second targeted modification is introduced using at least one second site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted codon deletion modification at the first plant genomic target site, and optionally by further selecting (ii) for the at least one second targeted modification in the second plant genomic target site, (d) optionally: crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

In a further aspect according to the present invention there is provided a method for isolating at least one modified plant cell or at least one modified tissue, organ, or whole plant comprising the at least one modified plant cell, without stably integrating a transgenic selectable marker sequence, the method comprising: (a) introducing at least one first targeted frameshift or deletion modification into a first plant genomic target site of at least one plant cell to be modified using at least one first site-specific effector, wherein the at least one targeted frameshift or deletion modification causes expression of at least one phenotypically selectable trait; (b) introducing at least one second targeted modification into a second plant genomic target site of the at least one plant cell to be modified, wherein the at least one second targeted modification is introduced using at least one second site-specific effector, comprising a nuclease, a recombinase, or a DNA modification reagent, to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or whole plant thereof comprising the at least one first targeted modification to obtain at least one modified plant cell; and (c) isolating at least one modified plant cell, tissue, organ, or whole plant, or isolating at least one progeny cell, tissue, organ, or plant thereof by selecting (i) for the at least one phenotypically selectable trait caused by the at least one first targeted frameshift or deletion modification at the first plant genomic target site, and optionally by further selecting (ii) for the at least one second targeted modification in the second plant genomic target site, (d) optionally: crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

As detailed above, the methods according to the present invention provide a new way of combining two different molecular complexes, one complex being configured to introduce at least one first targeted modification resulting in a selectable phenotype without inserting a transgenic marker, and the other complex configured to introducing at least one second targeted modification, wherein the first modification serves for screening purposes, whilst the second modification represents a genomic edit to be introduced. Therefore, the methods of the present invention synergistically combine genome editing strategies at different genomic target sites to achieve different targeted modifications ultimately resulting in an efficient breeding process to achieve a plant having a genotype of interest.

In certain embodiments, step b. of the methods of the present invention additionally comprises introducing a repair template (RT) to make a targeted sequence conversion or replacement at the at least one first and/or second plant genomic target site. This RT adds another level of precision to the genome editing approach, as the provision of a suitable RT, provided separately, or as part of at least one complex according to the present invention, as the break resulting from a nuclease or nickase can be repaired in a predetermined way by providing a RT of interest to assist homology-directed repair instead of relying on an error prone endogenous NHEJ pathway as repair mechanism. In one embodiment, a CRISPR-based nuclease is used as site-specific effector interacting with a gRNA, wherein the gRNA can be covalently linked to a RT, or wherein the CRISPR-based nuclease and/or the gRNA interact non-covalently with the RT. In another embodiment, the RT is provided separately, including addition on a construct encoding a RT of interest, and the RT will associate with a site-specific effector complex by means of complementary base pairing mediated by homology arms within the RT annealing to at least one genomic target site of interest.

In one embodiment a fusion protein or a non-covalently associated active Cpf1 and an inactive dCas9 as interaction domain can be provided as site-specific effector. The gRNA for Cas9 can target the repair template or an extension thereof, forming a Cpf1-dCas9-RT complex. The crRNA (Cpf1) targets the genomic locus defined for the double strand cut to initiate HDR. Likewise, a highly active zinc finger protein, a megaTAL or an inactive meganuclease can be used.

In one embodiment according to the various aspects of the present invention, a plant cell, tissue, organ, material or whole plant, or a progeny thereof, obtainable by any one of the methods disclosed herein is provided.

Due to the fact that the methods provided herein are specifically designed to assist in the provision of new plants having agronomically favorable traits, but do not comprise a transgenic marker sequence, the methods disclosed herein are suitable for creating a variety of different plant genotypes in a fast and reliable way.

In one embodiment according to the various aspects of the present invention, the at least one plant cell to be modified is preferably being derived from a plant selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

Method for Producing Genetically Modified Transgene-Free Plants:

In a further aspect, the present invention provides a method of generating a genetically modified plant by genome editing, the method comprising the steps of:
a) providing cells or tissues of the plant to be genetically modified;
b) providing a first genome editing system and a second genome editing system, wherein the first genome editing system can target and modify a plant selectable marker gene, and the second genome modification system can target and modify a gene of interest in the plant;
c) co-transforming the cells or tissues with the first and second genome editing systems;
d) regeneration plants from said transformed cells or tissues, preferably without selection pressure;
e) selecting plants in which the selectable marker gene has been modified from the plants regenerated in step d); and
f) identifying a plant whose target gene is modified from the plants selected in step e).

The cells or tissues of the plant include any cells or tissues that can be regenerated into intact plants, such as protoplasts, callus, explants, immature embryos, and the like.

As used herein, “genetical modification” includes altering the sequence of a gene and/or altering the expression of a gene.

As used herein, the term “gene of interest” means any nucleotide sequence to be modified in a plant, including both structural and non-structural genes. Preferably, the gene of interest is associated with a trait of the plant, preferably a agronomic trait.

As used herein, “selectable marker gene” means a plant endogenous gene that, after suitably modified, confers the plant a selectable trait that can be selected. Preferably, when suitably modified, the selectable marker gene does not substantially altering other traits of the plant.

For example, the selectable marker gene may be a plant endogenous herbicide resistance gene, which confer herbicide resistance to the plant when suitably modified. The plant endogenous herbicide resistance genes include but are not limited to PsbA, ALS, EPSPS, ACCase, PPO, and HPPD, PDS, GS, DOXPS, and P450. The ALS mutation sites capable of conferring herbicide resistance include, but are not limited to, A122, P197, A205, and S653 (the amino acid numbering refers to the amino acid sequence of the ALS in Arabidopsis thaliana). The EPSPS mutation sites capable of conferring herbicide resistance include, but are not limited to, T102, P106 (amino acid numbering refers to the EPSPS amino acid sequence in Arabidopsis thaliana). ACCase mutation sites capable of conferring herbicide resistance include, but are not limited to, I1781, W2027, I2041, D2078, and G2096 (amino acid numbering refers to the amino acid sequence of the chloroplast ACCase in Alopecurus myosuroides). HPPD mutation sites capable of conferring herbicide resistance include, but are not limited to, P277, L365, G417, and G419 (amino acid numbering refers to the amino acid sequence of the HPPD enzyme in rice).

In some embodiments of the invention, the ALS mutation site capable of conferring herbicide resistance in wheat includes TaALS P173. In some embodiments, the ALS mutation site capable of conferring herbicide resistance in corn includes ZmALS P165. In some embodiments, the ALS mutation site capable of conferring herbicide resistance in rice includes OsALS P171.

Alternatively, the selectable marker gene may be a gene that, when modified appropriately, causes the plant to produce visually-observable trait changes, such as genes controlling ligule, leaf color, leaf wax, including but not limited to LIG, PDS, zb7, and GL2.

Traditional methods of plant modification (transgenic methods) require the application of certain selective pressures during plant regeneration (eg, screening using different antibiotics depending on the transgene vector used) to increase the efficiency. However, this will lead to the integration of foreign genes, in particular antibiotic resistance genes, in the plant genome, resulting in potential safety issues.

By using the genome editing technology for plant modification, the genome editing system can achieve the target gene modification without integration into the plant genome. Thus, in the method of the invention, the regeneration of step d) is preferably carried out without selective pressure. This avoids the integration of foreign genes and results in genetically modified (genomically edited) transgenic plants. However, regeneration of plants without selective pressure will greatly reduce screening efficiency.

This problem is solved in the present invention by co-transforming a genome editing system that targets the gene of interest and a genome editing system that targets the endogenous selectable marker gene.

Without being bound by any theory, in the method of the present invention, a genome editing system that targets the gene of interest and a genome editing system that targets the endogenous selectable marker gene are co-transformed into a plant (such as a plant cell or tissue), then editing of the gene of interest and endogenous selectable marker genes will tend to occur together. Therefore, a plant selected based on an endogenous selectable marker gene will have a high probability that its gene of interest will also be modified. The first screen for the editing of endogenous selectable marker genes will greatly improve the screening efficiency of editing of the gene of interest. And, because only endogenous selectable marker genes are used, transgene concerns are avoided. In the present invention, the endogenous selectable marker gene preferably does not affect the trait of interest after being modified, for example, does not reduce yield and the like. More preferably, the modification of the endogenous selectable marker gene confers the plant additional traits of interest, such as herbicide resistance. That is, it is preferred that the traits available for selection of plants in the present invention are also agronomically useful traits such as herbicide resistance.

The method of performing the selection in step e) depends on the nature of the selectable marker gene. For example, if the selectable marker gene is modified to confer herbicide resistance to the plant, the regenerated plant can be placed at a suitable concentration at which the plant having the wild-type selectable marker gene cannot survive or grow poorly. Then, plants that survive or grow well at this concentration of herbicide are selected.

The identification in step f) can be performed by, for example, PCR/RE, or sequencing methods. The person skilled in the art is well acquainted with how to identify whether a gene has been mutated or not.

Suitable methods for transforming a plant (cell or tissue) of the present invention include, but are not limited to, particle bombardment, PEG-mediated protoplast transformation, and Agrobacterium-mediated transformation.

The present invention does not particularly limit to a specific genome editing system, as long as it enables accurate editing of the plant genome. For example, genome editing systems suitable for use with the present invention include, but are not limited to, precise base editor (PBE) systems, CRISPR-Cas9 systems, CRISPR-Cpf1 systems, CRISPRi systems, zinc finger nuclease systems, and TALEN systems. The choose or design of suitable genome editing systems that target the gene of interest and the endogenous selectable marker gene are within the skills of one skilled in the art.

CRISPR systems are produced by bacteria during evolution to protect against foreign gene invasion. It has been modified and widely used in genome editing of eukaryotes.

CRISPR-Cas9 system refers to a Cas9 nuclease-based genome CRISPR editing system. “Cas9 nuclease” and “Cas9” are used interchangeably herein and refer to an RNA Guided nuclease that include a Cas9 protein or fragment thereof (eg, a protein comprising the active DNA cleavage domain of Cas9 and/or the gRNA binding domain of Cas9). Cas9 is a component of the prokaryotic immune system of CRISPR/Cas that can target and cleave DNA target sequences to form DNA double-strand breaks (DSBs) under the guidance of guide RNA. CRISPR-Cas9 systems suitable for use in the present invention include, but are not limited to, those described in Shan, Q. et al. Targeted genome modification of crop plants using a CRISPR-Cas system. Nat. Biotechnol. 31, 686-688 (2013).

“guide RNA” and “gRNA” can be used interchangeably herein, which typically are composed of crRNA and tracrRNA molecules forming complexes through partial complement, wherein crRNA comprises a sequence that is sufficiently complementary to a target sequence for hybridization and directs the CRISPR complex (Cas9+crRNA+tracrRNA) to specifically bind to the target sequence. However, it is known in the art that single guide RNA (sgRNA) can be designed, which comprises the characteristics of both crRNA and tmcrRNA.

The CRISPR-Cas9 system of the present invention may include one of the following:

i) a Cas9 protein, and a guide RNA;

ii) an expression construct comprising a nucleotide sequence encoding a Cas9 protein, and a guide RNA;

iii) a Cas9 protein, and an expression construct comprising a nucleotide sequence encoding a guide RNA;

iv) an expression construct comprising a nucleotide sequence encoding a Cas9 protein, and an expression construct comprising a nucleotide sequence encoding a guide RNA; or

v) an expression construct comprising a nucleotide sequence encoding a Cas9 protein and a nucleotide sequence encoding a guide RNA.

The CRISPR-Cpf1 system is a CRISPR genome editing system based on the Cpf1 nuclease. The difference between Cpf1 and Cas9 is that the molecular weight of the Cpf1 protein is small, and only crRNA is required as the guide RNA, and the PAM sequence is also different. The CRISPR-Cpf1 system suitable for use in the present invention includes, but is not limited to, the system described in Tang et al., 2017.

The CRISPR-Cpf1 system of the present invention may include one of the following:

i) a Cpf1 protein, and a guide RNA (crRNA);

ii) an expression construct comprising a nucleotide sequence encoding a Cpf1 protein, and a guide RNA;

iii) a Cpf1 protein, and an expression construct comprising a nucleotide sequence encoding a guide RNA;

iv) an expression construct comprising a nucleotide sequence encoding a Cpf1 protein, and an expression construct comprising a nucleotide sequence encoding a guide RNA; or

v) an expression construct comprising a nucleotide sequence encoding a Cpf1 protein and a nucleotide sequence encoding a guide RNA.

CRISPR interference (CRISPRi) is a gene silencing system derived from the CRISPR-Cas9 system that uses a nuclease-inactivated Cas9 protein. Although this system does not change the sequence of the target gene, it is also defined herein as a genome editing system. CRISPRi systems suitable for use with the present invention include, but are not limited to, the system described in Seth and Harish, 2016.

The CRISPRi system of the present invention may include one of the following:

i) a nuclease-inactivated Cas9 protein, and a guide RNA;

ii) an expression construct comprising a nucleotide sequence encoding a nuclease-inactivated Cas9 protein, and a guide RNA;

iii) a nuclease-inactivated Cas9 protein, and an expression construct comprising a nucleotide sequence encoding a guide RNA;

iv) an expression construct comprising a nucleotide sequence encoding a nuclease-inactivated Cas9 protein, and an expression construct comprising a nucleotide sequence encoding a guide RNA; or

v) an expression construct comprising a nucleotide sequence encoding a nuclease-inactivated Cas9 protein and a nucleotide sequence encoding a guide RNA.

The precise base editor system is a system that has recently been developed based on CRISPR-Cas9, which enables accurate single-base editing of a genome using a nuclease-inactivated fusion protein of Cas9 protein and cytidine deaminase. Nuclease-inactivated Cas9 (due to mutations in the HNH subdomain and/or RuvC subdomain of the DNA cleavage domain) retains gRNA-directed DNA-binding ability, and the cytidine deaminase can catalyze deamination of cytidine(C) on DNA to form uracil (U). The nuclease-inactivated Cas9 is fused with a cytidine deaminase. Under the guidance of the guide RNA, the fusion protein can target the target sequence in the plant genome. Due to the absence of the Cas9 nuclease activity, the DNA double strand is not cleaved. The deaminase domain in the fusion protein converts the cytidine of the single-stranded DNA produced in the formation of the Cas9-gRNA-DNA complex to U, and the substitution of C to T is achieved by base mismatch repair. The precise base editor system suitable for use in the present invention includes, but is not limited to, the system described in Zong et al., 2017.

The precise base editor system of the present invention may include one of the following:

i) a fusion protein of nuclease-inactivated Cas9 and cytidine deaminase, and guide RNA;

ii) an expression construct comprising the nucleotide sequence encoding a fusion protein of a nuclease-inactivated Cas9 protein and a cytidine deaminase, and a guide RNA;

iii) a fusion protein of nuclease-inactivated Cas9 protein and cytidine deaminase, and an expression construct comprising a nucleotide sequence encoding a guide RNA;

iv) an expression construct comprising a nucleotide sequence encoding a fusion protein of a nuclease-inactivated Cas9 protein and a cytidine deaminase, and an expression construct comprising a nucleotide sequence encoding a guide RNA; or

v) an expression construct comprising a nucleotide sequence encoding a fusion protein of a nuclease-inactivated Cas9 protein and a cytidine deaminase and a nucleotide sequence encoding a guide RNA.

In some embodiments, the nuclease-inactivated Cas9 protein comprises amino acid substitutions D 10A and/or H840A relative to wild-type Cas9 (S. pyogenes SpCas9). Examples of the cytidine deaminase include, but are not limited to, APOBEC1 deaminase, activation-induced cytidine deaminase (AID), APOBEC3G, or CDA1(PmCDA1).

“Zinc finger nuclease (ZFN)” is an artificial restriction enzyme prepared by fusing a zinc finger DNA binding domain with a DNA cleavage domain. The zinc finger DNA binding domain of a single ZFN typically contains 3-6 individual zinc finger repeats, each zinc finger repeat recognizing, for example, 3 bp. ZFN systems suitable for use in the present invention can be obtained, for example, from Shukla et al., 2009 and Townsend et al., 2009.

“Transactivator-like effector nucleases (TALENs)” are restriction enzymes that can be engineered to cleave specific DNA sequences, usually prepared by fusion of the DNA binding domain of the transcriptional activator-like effector (TALE) and a DNA cleavage domain. TALE can be engineered to bind almost any desired DNA sequences. The TALEN system suitable for use in the present invention can be obtained, for example, from Li et al., 2012.

Those skilled in the art can appropriately determine the combination of the first genome editing system and the second genome editing system in the method of the present invention according to the respective characteristics of different genome editing systems and the specific type of genome editing desired to be implemented, for example, selecting a suitable combination to avoid interference with each other, for example, interference between different systems that can share a same gRNA.

For example, if the endogenous selectable marker gene requires a single base editing system for precise mutation to generate selectable traits, the CRISPR-Cas9 system is generally not used to target the gene of interest because the two systems can share a same gRNA and thus Cas9 for knockout of the gene of interest may also knock out the endogenous selectable marker gene, vice versa.

In some preferred embodiments of the methods of the present invention, wherein both the first and second genome editing systems are precise base editor systems.

In some embodiments of the invention, the components of the first and second genome editing systems may be expressed by the same expression construct or by different expression constructs, which can be conveniently selected by those skilled in the art. For example, guide RNAs for a gene of interest and a selectable marker gene can be transcribed with the same expression construct. Preferably, the components of the first and second genome editing systems are expressed by the same expression construct.

In some embodiments of the method of the present invention, wherein the first and second genome editing systems are both precise base editor systems, and wherein fusion protein of nuclease-inactivated Cas9 protein and cytidine deaminase and guide RNAs for gene of interest and the selectable marker gene are expressed by a same expression construct.

In some embodiments of the method of the present invention, the plant is monocotyledonous or dicotyledonous. For example, the plant is selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Hehanthus tuberosus and Allium tuberosum, or any variety or subspecies belonging to one of the aforementioned plants. In some embodiments, the plant is a crop plant.

In some embodiments of the invention, the method further comprises obtaining progeny of the genetically modified transgene-free plant.

In another aspect, the present invention also provides a genetically modified plant or a progeny thereof or a part thereof, wherein the plant is obtained by the above method of the present invention.

In another aspect, the present invention also provides a plant breeding method comprising crossing a first genetically modified plant obtained by the above method of the present invention with a second plant not containing the genetic modification, thereby introducing said genetic modification into the second plant.

By simultaneously targeting the gene of interest to be modified and the endogenous selectable marker gene in the plant, the screening efficiency of genetically modified transgene-free plants can be greatly improved. By the method of the present invention, the screening efficiency of transgene-free mutants can be improved by about 10-100 times for a gene of interest having a mutation rate of less than 1%.

Delivery Methods:

A variety of suitable delivery techniques for introducing genetic material into a plant cell are known to the skilled person., e.g. by choosing direct delivery techniques ranging from polyethylene glycol (PEG) treatment of protoplasts (Potrykus et al. 1985), procedures like electroporation (D′Halluin et al., 1992), microinjection (Neuhaus et al., 1987),silicon carbide fiber whisker technology (Kaeppler et al., 1992), viral vector mediated approaches (Gelvin, Nature Biotechnology 23, “Viral-mediated plant transformation gets a boost”, 684-685 (2005)) and particle bombardment (see e.g. Sood et al., 2011, Biologia Plantarum, 55, 1-15).

Despite transformation methods based on biological approaches, like Agrobacterium transformation or viral vector mediated plant transformation, and methods based on physical delivery methods, like particle bombardment or microinjection, have evolved as prominent techniques for introducing genetic material into a plant cell or tissue of interest. Helenius et al. (“Gene delivery into intact plants using the Helios™ Gene Gun”, Plant Molecular Biology Reporter, 2000, 18 (3):287-288) discloses a particle bombardment as physical method for introducing material into a plant cell. Currently, there thus exists a variety of plant transformation methods to introduce genetic material in the form of a genetic construct into a plant cell of interest, comprising biological and physical means known to the skilled person on the field of plant biotechnology and which can be applied to introduce the at least on base editor and the at least one site-specific effector as well as the corresponding complexes comprising the at least on base editor and the at least one site-specific effector. Notably, said delivery methods for transformation and transfection can be applied to introduce the tools of the present invention simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alfa transfection with calcium phosphate, transfection using liposomes, .e.g., cationic liposomes, or transfection with cationic polymers, including DEAD-dextran or polyethylenimine, or combinations thereof. Said delivery methods and delivery vehicles or cargos thus inherently differ from delivery tools as used for other eukaryotic cells, including animal and mammalian cells and every delivery method has to be specifically fine-tuned and optimized so that a construct of interest for mediating genome editing can be introduced into a specific compartment of a target cell of interest in a fully functional and active way. The above delivery techniques, alone or in combination, can be used to insert the at least one molecular complex according to the present invention, i.e., a base editor complex and/or a site-specific effector complex, or at least one subcomponent thereof, i.e., at least one SSN, at least one gRNA, at least one RT, or at least one base editor, or the sequences encoding the aforementioned subcomponents, according to the present invention into a target cell, in vivo or in vitro.

Physical and chemical delivery methods are particularly preferred according to the present invention, as said methods allow the co-delivery and thus the parallel introduction of various tools of interest into at least one plant cell.

In certain embodiments, the crRNA portion of the gRNA comprises a stem loop or an optimized stem loop structure or an optimized secondary structure. In another embodiment the mature crRNA comprises a stem loop or an optimized stem loop structure in the direct repeat sequence, wherein the stem loop or optimized stem loop structure is important for cleavage activity. In certain embodiments, the mature crRNA preferably comprises a single stem loop. In certain embodiments, the direct repeat sequence preferably comprises a single stem loop. In certain embodiments, the cleavage activity of the effector protein complex is modified by introducing mutations that affect the stem loop RNA duplex structure. In preferred embodiments, mutations which maintain the RNA duplex of the stem loop may be introduced, whereby the cleavage activity of the effector protein complex is maintained. In other preferred embodiments, mutations which disrupt the RNA duplex structure of the stem loop may be introduced, whereby the cleavage activity of the effector protein complex is completely abolished.

Notably, the methods according to the various aspects of the present invention are not restricted to a first and/or second targeted modification being a modification within a coding region encoding an amino acid. The modification of a regulatory sequence is envisaged as well. Any modification having an epigenetic effect can also be addressed by the methods of the present invention.

In one embodiment the at least one genomic target sequence to be modified can be a regulatory sequence such as a promoter wherein the editing of the promoter comprises replacing the promoter, or promoter fragment with a different promoter (also referred to as replacement promoter) or promoter fragment (also referred to as replacement promoter fragment), wherein the promoter replacement results in any one of the following or any one combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer, for example, extending the timing of gene expression in the tapetum of anthers, a mutation of DNA binding elements and/or a deletion or addition of DNA binding elements. The promoter (or promoter fragment) to be modified can be a promoter (or promoter fragment) that is endogenous, artificial, pre-existing, or transgenic to the cell that is being edited. The replacement promoter or fragment thereof can be a promoter or fragment thereof that is endogenous, artificial, pre-existing, or transgenic to the cell that is being edited.

In one embodiment the at least one genomic target sequence can be a promoter wherein the editing of the promoter comprises replacing a native EPSPS1 promoter from with a plant ubiquitin promoter. In another embodiment the at least one genomic target sequence to be modified can be a promoter wherein the promoter to be edited is selected from the group comprising a Zea mays-PEPC1 promoter (Kausch et al., Plant Molecular Biology, 45: 1-15, 2001), a Zea mays ubiquitin promoter (UBIlZM PRO, Christensen et al., plant Molecular Biology 18: 675-689, 1992), a rice actin promoter (McElroy et al., The Plant Cell, Vol 2, 163-171, February 1990), a Zea mays-GOS2 promoter (U.S. Pat. No. 6,504,083), or a Zea mays oleosin promoter (U.S. Pat. No. 8,466,341).

In one embodiment, the at least one site-specific effector complex can be used in combination with a co-delivered RT to allow for the insertion of a promoter or promoter element into a genomic nucleotide sequence of interest without incorporating a selectable transgene marker, wherein the promoter insertion (or promoter element insertion) results in any one of the following or any one combination of the following: an increased promoter activity. i.e., increased promoter strength, increased promoter tissue specificity, a decreased promoter activity, a decreased promoter tissue specificity, a new promoter activity, an inducible promoter activity, an extended window of gene expression, a modification of the timing or developmental progress of gene expression a mutation of DNA binding elements and/or an addition of DNA binding elements. Promoter elements to be inserted can be, but are not limited to, promoter core elements, such as, but not limited to, a CAAT box, a CCAAT box, a Pribnow box, a and/or TATA box, translational regulation sequences and/or a repressor system for inducible expression, such as TET operator repressor/operator/inducer elements, or sulphonylurea repressor/operator/inducer elements. The dehydration-responsive element (DRE) was first identified as a cis-acting promoter element in the promoter of the drought-responsive gene rd29A, which contains a 9 bp conserved core sequence, TACCGACAT (Yamaguchi-Shinozaki, K., and Shinozaki, K. (1994) Plant Cell 6, 251-264). Insertion of DRE into an endogenous promoter may confer a drought inducible expression of the downstream gene. Another example is ABA-responsive elements (ABREs) which contains a (C/T)ACGTGGC consensus sequence found to be present in numerous ABA and/or stress-regulated genes (Busk P. K., Pages M. (1998) Plant Mol. Biol. 37:425-435). Insertion of 35S enhancer or MMV enhancer into an endogenous promoter region will increase gene expression (U.S. Pat. No. 5,196,525). The promoter, or promoter element, to be inserted can be a promoter, or promoter element, that is endogenous, artificial, pre-existing, or transgenic to the cell that is being edited.

In one embodiment, the at least one site-specific effector complex can be used to insert an enhancer element, such as but not limited to a Cauliflower Mosaic Virus 35 S enhancer, in front of an endogenous FMT1 promoter to enhance expression of the FTM1. In a further embodiment, the at least one site-specific effector complex can be used to insert a component of the TET operator repressor/operator/inducer system, or a component of the sulphonylurea repressor/operator/inducer system into plant genomes to generate or control inducible expression systems without incorporating a selectable transgene marker.

In another embodiment, the at least one site-specific effector complex can be used to allow for the deletion of a promoter or promoter element, wherein the promoter deletion (or promoter element deletion) results in any one of the following or any one combination of the following: a permanently inactivated gene locus, an increased promoter activity (increased promoter strength), an increased promoter tissue specificity, a decreased promoter activity, a decreased promoter tissue specificity, a new promoter activity, an inducible promoter activity, an extended window of gene expression, a modification of the timing or developmental progress of gene expression, a mutation of DNA binding elements and/or an addition of DNA binding elements. Promoter elements to be deleted can be, but are not limited to, promoter core elements, promoter enhancer elements or 35S enhancer elements. The promoter or promoter fragment to be deleted can be endogenous, artificial, pre-existing, or transgenic to the cell that is being edited.

In yet another embodiment, the at least one genomic target site of interest to be modified can be a terminator wherein the editing of the terminator comprises replacing the terminator, also referred to as a “terminator swap” or “terminator replacement”, or terminator fragment with a different terminator, also referred to as replacement terminator, or terminator fragment, also referred to as replacement terminator fragment, wherein the terminator replacement results in any one of the following or any one combination of the following: an increased terminator activity, an increased terminator tissue specificity, a decreased terminator activity, a decreased terminator tissue specificity, a mutation of DNA binding elements and/or a deletion or addition of DNA binding elements. The terminator or fragment thereof to be modified can be a terminator that is endogenous, artificial, pre-existing, or transgenic to the cell that is being edited. The replacement terminator can be a terminator or fragment thereof that is endogenous, artificial, pre-existing, or transgenic to the cell that is being edited.

In one embodiment the at least one genomic target site of interest to be modified can be a terminator wherein the terminator to be edited is selected from the group comprising terminators from maize Argos 8 or SRTF18 genes, or other terminators, such as potato PinII terminator, sorghum actin terminator (WO 2013/184537 A1), rice T28 terminator (WO 2013/012729 A2), AT-T9 TERM (WO 2013/012729 A2) or GZ-W64A TERM (U.S. Pat. No. 7,053,282).

In one embodiment, the at least one site-specific effector complex according to the present invention can be used in combination with a co-delivered RT sequence to allow for the insertion of a terminator or terminator element into a genomic nucleotide sequence of interest, wherein the terminator (element) insertion results in any one of the following or any one combination of the following: an increased terminator activity, i.e., increased terminator strength, an increased terminator tissue specificity, a decreased terminator activity, a decreased terminator tissue specificity, a mutation of DNA binding elements and/or an addition of DNA binding elements.

The terminator or element or fragment thereof to be inserted can be a terminator (or terminator element) that is endogenous, artificial, pre-existing, or transgenic to the cell that is being edited.

In yet another embodiment, the at least one site-specific effector complex can be used to allow for the deletion of a terminator or terminator element, wherein the terminator deletion (or terminator element deletion) results in any one of the following or any one combination of the following: an increased terminator activity (increased terminator strength), an increased terminator tissue specificity, a decreased terminator activity, a decreased terminator tissue specificity, a mutation of DNA binding elements and/or an addition of DNA binding elements. The terminator or terminator fragment to be deleted can be endogenous, artificial, pre-existing, or transgenic to the cell that is being edited.

In one embodiment, the at least one site-specific effector complex of the present invention can be used to modify or replace a regulatory sequence in the genome of a cell without incorporating a selectable transgene marker. A regulatory sequence is a segment of a nucleic acid molecule which is capable of increasing or decreasing the expression of specific genes within an organism and/or is capable of altering tissue specific expression of genes within an organism. Examples of regulatory sequences include, but are not limited to, 3′ UTR (untranslated region) region, 5′ UTR region, transcription activators, transcriptional enhancers transcriptions repressors, translational repressors, splicing factors, miRNAs, siRNA, artificial miRNAs, promoter elements, CAMV 35 S enhancer, MMV enhancer elements, SECIS elements, polyadenylation signals, and polyubiquitination sites. In some embodiments the editing in the form of at least one targeted modification of the present invention, or the replacement of a regulatory element results in altered protein translation, RNA cleavage, RNA splicing, transcriptional termination or post translational modification. In one embodiment, regulatory elements can be identified within a promoter and these regulatory elements can be edited or modified do to optimize these regulatory elements for up or down regulation of the promoter.

In one embodiment, the at least one genomic target site of interest to be modified is a polyubiquitination site, wherein the modification of the polyubiquitination sites results in a modified rate of protein degradation. The ubiquitin tag condemns proteins to be degraded by proteasomes or autophagy. Proteasome inhibitors are known to cause a protein overproduction. Modifications made to a DNA sequence encoding a protein of interest can result in at least one amino acid modification of the protein of interest, wherein said modification allows for the polyubiquitination of the protein (a post translational modification) resulting in a modification of the protein degradation.

In a further embodiment, the at least one genomic target site of interest to be modified is a polyubiquitination site on a maize EPSPS gene, wherein the polyubiquitination site modified resulting in an increased protein content due to a slower rate of EPSPS protein degradation.

In yet a further embodiment, the at least one genomic target site of interest to be modified is a an intron site, wherein the modification consist of inserting an intron enhancing motif into the intron which results in modulation of the transcriptional activity of the gene comprising said intron.

The present invention will now be illustrated by the following Examples, which are not construed to limit the scope of the present invention.

### EXAMPLES

### Example 1

### Next Generation Sequencing to Verify Base Editing

To test the activity using the base editor coupled nickase for the targets described earlier, a plasmid encoding APOBEC-XTEN-Cas9 (nickase)-UGI (SEQ ID NO:1 and SEQ ID NO:2) was constructed by standard methods and the base editor and sgRNA were transiently expressed in cells derived from Zea mays tissues. Together with the complex, gRNAs designed for examples 2 to 6 were tested. Furthermore, specific PAM motifs (see SEQ ID NOs:3 to 13 and 23) were defined in relation to a target site of interest.

In addition, to increase the range of target sites available for conversion of the relevant amino acids in certain herbicide target genes, the SaKKH-BE3 and VQR-BE3 proteins (Komor A. et al., Increasing the genome-targeting scope and precision of base editing with engineered Cas9-cytidine deaminase fusion, Nat. Biotech. (2017)) was be codon-optimized for expression in corn, synthesized, and cloned into a plasmid together with the appropriate sgRNAs for expression in the same corn cell systems.

Total genomic DNA was extracted from the cell populations 12-96 hours after treatment with the base editor expressing plasmids and subjected to targeted deep sequencing to analyze the frequency and pattern of base conversions at the targets. The ability to make conversions causing herbicide-resistant amino acid substitutions in ALS1 (especially P197, S653), ALS2 (especially P197, S653), and PPO (especially C215, A220, G221, N425, Y426) genes was assessed.

### Example 2

### Transformation of Base Editing Components and Selection Against Sulfonylureas or Imidazolinones

To demonstrate the feasibility of the base editing to confer herbicide resistance using the methods described in this document, the base editors described in Example 1, and using several specifically designed gRNAs targeted to the corn ALS1, ALS2 genes that were validated by NGS in Example 1 were transformed into tissues from Zea mays and regenerated on selection media either containing a sulfonylurea (for P197 or S653 substitutions) or an imidazolinone (for S653 substitutions). A herbicide resistant plant will have undergone a base conversion due to the action of the base editor, resulting in a substitution of the proline in position 197 or the serine in position 653, depending on which base editor was delivered. To verify the base conversion event, the ALS genes in herbicide resistant plants was selected using the complementary herbicide and it was analyzed using molecular techniques.

### Example 3

### Co-Selection for Herbicide Resistance Due to Action of a Base Editor to Enrich the Frequency of a Non-Selectable Modification at an Unlinked Locus

To demonstrate that the transgene-free selection for isolating plants with gene editing events provides a suitable and straightforward tool during genome engineering, the methodology described in Example 2 was combined with the co-delivery of a site-specific nuclease to simultaneously generate base-conversions of a herbicide gene and targeted modifications of a gene of interest in the same cell in parallel. On the same plasmid, or a second plasmid, a nuclease is encoded together with a sgRNA and optionally a repair template to make a targeted modification in the same cells undergoing a base conversion due to the action of the base editor. At a later stage, plants can be regenerated under herbicide selection as described in Example 2, and then screened by molecular and other appropriate techniques for targeted modifications at the gene of interest, whereas the herbicide selection allows a significant decrease in the number of cells to be screened for the at least one second modification, i.e., the at least one targeted modification at the second genomic locus representing the gene of interest to be modified.

### Example 4

### Design of a Functional CRISPR/Cpf1 Base editor and Definition of the Base Editing Window

In this example, a second CRISPR protein, Cpf1, was used to deliver a base editor complex to the genomic target. Like CRISPR/Cas9, CRISPR/Cpf1 also forms an R loop like structure when binding its DNA target, leaving the non-target strand available in single-strand form for base conversions. However, because the exact position of the base conversion window with a Cpf1-derived base editor is unknown, it is necessary to analyze the base conversion pattern with respect to the PAM sequence in the target. The base conversion window can be defined by targeted NGS on GC-rich sequences of the corn genome, after delivery of Cpf1 based editors targeted to those sequences in cell populations as described in Example 1. For other target plants, the strategy can be adapted accordingly.

### Example 5

### Use of a Single-Nucleotide Deletion in a PPO Gene to Produce a Selectable Modification without a Repair Template or Homologous Recombination

A single amino acid deletion of glycine at position 210 of the PPO gene in Amaranthus tuberculatus has rendered this weed resistant to PPO-inhibiting herbicides (Patzoldt, W. L. et al. (2006). “A codon deletion confers resistance to herbicides inhibiting protoporphyrinogen oxidase” PNAS 103(33):12329-12334). This isoform is also called PPX2L. The equivalent amino acid in Nicotiana tabacum is a glycine in position 178 of the PPO2 gene. In Zea mays, the equivalent amino acid is an alanine, but the surrounding residues are highly conserved and likely still constitute a functional active site that would become resistant due to deletion of the alanine.

In this example, a site-directed nuclease such as Cas9 or Cpf1 can be used with appropriate crRNA or sgRNA to make a double-strand cut near the codon for this amino acid. Three-base deletions that preserve an active PPO enzyme while inhibiting herbicide binding will result in herbicide resistant plants. Thus, this selectable modification can be made without the use of a repair template or homologous recombination thus providing a transgenic marker free strategy.

### Example 6

### Additional Applications

Additional examples are conceivable using the CRISPR nucleases CasX, CasY, and Cpf1 together with the applications described for CRISPR Cas9 in Examples 1-3 above. Additionally, the introduction of early stop codons using the Cas9-linked base editor described in Example 1 or the base editor linked to Cpf1 as described in Example 4 into selectable gene targets or phenotypic markers for plant screening. Specific examples can be stop codons in phenotypic genes (e.g., the many glossy genes, golden, etc).

Further targets for selection based on herbicide-resistance also include other amino acid deletions, introduction of early stop codons, or amino acid changes in the PPO, ALS, and EPSPS genes as described earlier. gRNA protospacer sequences suitable for base editing in the PPO gene are provided (see SEQ ID NOs:7 to 13).

Further provided is a sequence for a CasX-linked base editing complex (SEQ ID NO:14), a sequence for a AsCpf1-linked base editing complex (SEQ ID NO:15), and a sequence for incorporation of the cytidine deaminase PmCDA1 into a Cas9-linked base editing complex (SEQ ID NO:16).

For optimization, and particularly for de novo design of CRISPR nuclease-linked base editing complexes, any order and combination of the following components can be used: niCas9 (D10A; SEQ ID NO:17), CasX (SEQ ID NO:18), niAsCpf1 (R1226A; SEQ ID NO:19), APOBEC1 (SEQ ID NO:20), UGI (SEQ ID NO:21), PmCDA1 (SEQ ID NO:22), as well as linkers, including XTEN linkers, and nuclear localization signals or other organelle targeting signals depending on the genomic site of interest, or any combination of the aforementioned components.

### Example 7

### Screening of Rice Mutant Plants

According to Yuan Zong et al. (Zong, Y. et al. Precise base editing in rice, wheat and maize with a Cas9-cytidine deaminase fusion. Nat. Biotechnol. 2017, doi: 10.1038/nbt.3811), vector pH-nCas9-PBE-OsALS-S1/S2 that simultaneously targets two different sites (S1 and S2) of the OsALS gene (Genbank No.: AY885674.1) was constructed based on pH-nCas9-PBE. The S1 site of OsALS is used as a site for herbicide selection. If the S1 locus is mutated, the plants will gain resistance to herbicides such as nicosulfuron (Tranel and Wright, 2002). The sgRNA target sequence in the experiment is shown in Table 1.

TABLE 1
Rice sgRNA Target sequence

| sgRNA | Target sequence |
|---|---|
| sgRNA-OsALS-S1 | CAGGTCCCCCGCCGCATGATCGG |
| sgRNA-OsALS-S2 | CCTACCCGGGCGGCGCGTCCATG |

PAM is underlined.

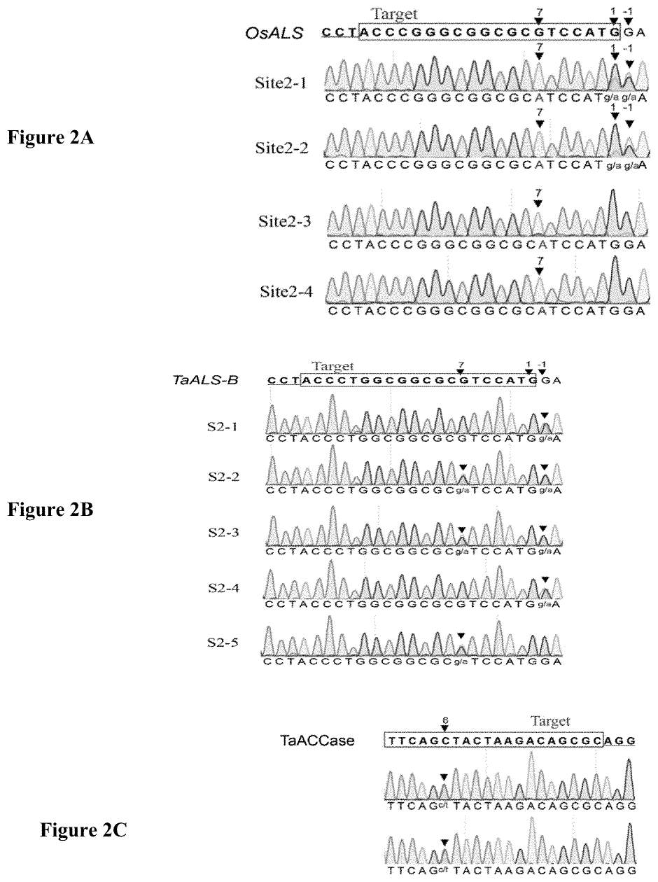

The pH-nCas9-PBE-OsALS-S1/S2 binary vector was transformed into Agrobacterium strain AGL1 by electroporation. Agrobacterium-mediated transformation, tissue culture and regeneration in rice cultivar Zhonghua 11 were performed according to Shan et al. (Shan, Q. et al. Targeted genome modification of crop plants using a CRISPR-Cas system. Nat. Biotechnol. 31, 686-688 (2013)). Hygromycin selection (50 μg/ml) was used during tissue culture. (This experiment is a proof of concept, so the plants were selected with hygromycin first, then by nicosulfuron. The objective was to first obtain transgenic plants and then screen for herbicide resistance). After regeneration of rice plants, 10 regenerated seedlings were grown on a selection medium containing 0.0065 PPM Nicosulfuron at which wild-type plants cannot survive. Four seedlings survived after 14 days. DNA was extracted from the four seedlings. The ALS gene was amplified by PCR, sequenced to determine the mutant genotype. The results showed that all four seedlings had base mutations at the S2 locus, and the herbicide-resistant plants had a mutation rate of 100% (4/4) at S2 site. The mutation pattern is shown in FIG. 2A.

### Example 8

### Screening of Wheat Mutant Plants

According to Yuan Zong et al. (Zong, Y. et al. Precise base editing in rice, wheat and maize with a Cas9-cytidine deaminase fusion. Nat. Biotechnol. 2017, doi: 10.1038/nbt.3811), the following constructs were prepared based on pTaU6:

1) pTaU6-TaALS-S2 targeting the S2 site of TaALS gene of B genome (GenBank No.: AY210406),

2) pTaU6-TaACCase targeting a site in TaACCase gene of B genome and D genome (Genbank No. EU660901 and EU660902),

3) pTaU6-TaALS-S1/S2 that targets two sites of the TaALS gene in parallel, and

4) pTaU6-TaALS-S1/TaACCase that targets TaALS and TaACCase genes in parallel.

TaALS S1 site was used as a herbicide selection site. If the site is mutated, plants will gain herbicide (such as nicosulfuron) resistance, while only mutation in TaALS S2 site will not confer resistance (Tranel and Wright, 2002). The sgRNA target sequence used in the experiment is shown in Table 2.

TABLE 2
Wheat sgRNA target sequence

| sgRNA | Target sequence |
|---|---|
| sgRNA-TaALS-S1 | CAGGTCCCCCGCCGCATGATCGG |
| sgRNA-TaALS-S2 | CCTACCCTGGCGGCGCGTCCATG |
| sgRNA-TaACCase | TTCAGCTACTAAGACAGCGCAGG |

PAM is underlined.

Plasmid DNA (mixture of equal proportions of pnCas9-PBE and pTaU6 vector series) was used to bombard young embryos of Konun 199 as previously described for transformation (Zhang, K., Liu, J., Zhang, Y., Yang, Z. & Gao, C. Biolistic genetic transformation of a wide range of Chinese elite wheat (Triticum aestivum L.) varieties. J. Genet. Genomics. 42, 39-42 (2015). After bombardment, embryos were processed according to the literature and no selective agent was used during tissue culture.

For wheat plants obtained by solely targeting the S2 site of the TaALS gene of B genome, every 3-4 plants were pooled as one sample to detect mutations by PCR/RE. 258 samples were detected by PCR/RE (approximately 1000 individual plants) and no mutation was detected.

For wheat plants obtained by solely targeting the site of TaACCase gene, every 3-4 plants were pooled as one sample and subjected to Sanger sequencing. 64 samples (about 256 individual plants) were sequenced, and no mutation was detected.

Wheat plants (approximately 800 plants) obtained by targeting the TaALS gene S1 and S2 sites in parallel were first grown on a selection medium containing 0 13 PPM Nicosulfuron (on which wild-type plants were unable to survive). 30 days later, twelve seedlings survived, and 9 of them had base mutations at the TaALS-S2 site. The efficiency of selecting the ALS-S2 site mutant plants using nicosulfuron selection medium was 75% (9/12). The mutation types of five mutants are shown in FIG. 2B.

Wheat plants (about 800 plants) obtained by targeting the TaALS and TaACCase genes in parallel were grown on a selection medium containing 0.13 PPM Nicosulfuron. After 30 days, 9 seedlings survived, and 2 plants had base mutations at the TaACCase site. The efficiency of selecting TaACCase site-mutant plants using nicosulfuron selection medium was 22% (2/9). The mutation pattern of the TaACCase site was shown in FIG. 2C.

The experimental results show that for the target gene whose mutation rate is very low (eg, the target gene has a mutation rate of 0.5%), the method of the invention can increase the probability of obtaining target mutation by 10-100 times.

### Example 9

### Development of Base Co-Editing System in Wheat Based on TaALS-P173

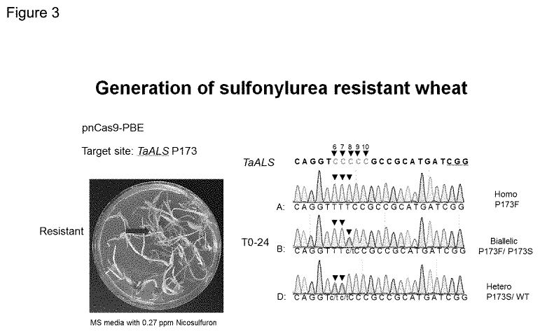

In this study, the sgRNA site corresponding to TaALS-P173 was used to establish the herbicide selection system during wheat transformation. PnCas9-PBE and TaALS-P173-sgRNA constructs were delivered into 640 immature embryo cells of the bread wheat variety Kenong 199 by particle bombardment. After seedlings (2-3 cm high) were regenerated, PCR restriction enzyme digestion assay (PCR-RE assay) was used to analyze the mutation frequency. Simultaneously, the same seedlings were transfer to the media containing 0.27 ppm nicosulfuron (FIG. 3). Ten (1.56%) out of fourteen (2.1%) mutant seedlings which are identified using PCR-RE assay showed resistance after 3 weeks growth on the herbicide containing media, and three sensitive mutants did not contain any amino acid substitution (Table 3).

TABLE 3

| | | A genome | B genome | D genome |
|---|---|---|---|---|
| Resistance | | substitution | substitution | substitution |
| R | T0-1 | F/S | F/S | F/S |
| R | T0-2 | F(hetero) | F(homo) | S(homo) |
| R | T0-3 | S(hetero) | F/S | S(hetero) |
| S | T0-4 | WT | WT | SM |
| R | T0-5 | S(homo) | S(hetero) | F/S |
| R | T0-6 | S(homo) | F, C(hetero) | WT |
| S | T0-7 | S(homo) | F, C(hetero) | WT |
| R | T0-8 | WT | WT | F(hetero) |
| R | T0-9 | F(homo) | F/S | S(hetero) |
| R | T0-10 | F(homo) | F/S | S(hetero) |
| R | T0-11 | S(hetero) | F(hetero) | F/S |
| R | T0-12 | S(hetero) | F(hetero) | F/S |
| S | T0-13 | WT | SM | WT |
| S | T0-14 | WT | SM | WT |

SM: Silent mutation;
S: Sensitive;
R: Resistant;
Homo: homozygous;
Hetero: heterozygous

The results confirmed that TaALS-P173 substitution can be recognized from herbicide containing media. The inventors then tested whether this site can also be used to select for other genome edited events. So three other sites (TaALS-A98, TaALS-A181, as well as TaACCase-A2004) were combined with TaALS-P173 separately. To evaluate the selection efficiency, the regenerated seedlings co-bombarded with TaALS-P173 site targeting systems were place on media containing nicosulfuron and the survived seedlings were submitted for genotyping. Targeted mutants were detected at all three sites (Table 4) at selection efficiencies up to 78%. In sites TaALS-A181 and TaACCase-A2004, the selection efficiencies were relative low (˜25%), which was possibly caused by the low conversion ability of deaminase APOBEC1 at GC context.

To increase the selection efficiency on sites with GC context, APOBEC1 was replaced by another deaminase-PmCDA1, which has different sequence preference compared with APOBEC1. Newly generated base editor pPmCDA1-PBE, TaACCase-A2004-sgRNA and TaALS-P173-sgRNA constructs were delivered into 640 immature embryo cells by particle bombardment. Out of 2 survived seedlings, both (100%) contained mutant alleles at target site TaACCase-A2004 (Table 4).

TABLE 4

| | | No. of | No. of | Selection |
|---|---|---|---|---|
| | | survived | mutants on | efficiency |
| | Deaminase | plants | second site | (%) |
| TaALS-P173 + | APOBEC1 | 18 | 14 | 78 |
| A98 | | | | |
| TaALS-P173 + | APOBEC1 | 8 | 2 | 25 |
| A181 | | | | |
| TaALS-P173 + | APOBEC1 | 9 | 2 | 22 |
| TaACCase A2004 | PmCDA1 | 2 | 2 | 100 |

### Example 10

### Development of Base Co-Editing System in Corn Based on ZmALS-P165

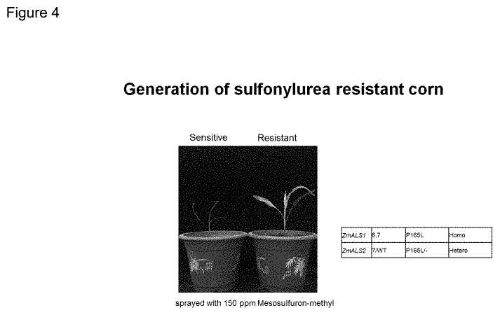

To establish the co-editing system in corn, acetolactate synthase site corresponding TaALS-P173 was targeted to test the herbicide resistance. It has been reported single edited allele on ZmALS2 could confer plants herbicide resistance (Svitashev et al, 2016). So the binary vector targeting ZmALS-P165 was transformed to immature embryos (ZmALS-P165 site is conserved in both ZmALS1 and ZmALS2). Three independent mutants were obtained from the regenerated plants and their genotypes are same. Two ZmALS1 alleles and one ZmALS2 allele containing C to T substitutions resulted in the single amino acid residue change: proline to leucine at position 165. One mutant plant with heterozygous P165L substitution on ZmALS2 showed resistance to Mesosulfuron-methyl, a sulfonylurea class of herbicides (FIG. 4).

After confirming ZmALS-P165 site could work well as a selectable marker, other two sites—ZmAccase A2004 and ZmSbe2 Stop, were combined with this selectable site separately. Both biolistic and Agrobacterium-mediated delivery were used for transformation. As ZmAccase A2004 site was within GC context, PmCDA1 was used to replace APOBEC1.

To evaluate the selection efficiency using biolistic delivery, bombarded calli as well as Agrobacterium transformed immature embryos were placed on Mesosulfuron-methyl containing medium. Surviving seedling showed target site mutation.

### Example 11

### Development of Base Co-Editing System in Rice Based on OsALS-P171

To establish the co-editing system in rice, acetolactate synthase site corresponding TaALS-P173 was targeted to test the herbicide resistance. It has been reported single edited allele could confer plants herbicide resistance (Kawai, K., Kaku, K., Izawa, N., Shimizu, M., Kobayashi, H., & Shimizu, T. (2008). Herbicide sensitivities of mutated enzymes expressed from artificially generated genes of acetolactate synthase. Journal of pesticide science, 33(2), 128-137.). So the binary vector targeting OsALS-P171 was transformed to immature embryos. Mutants were obtained from the regenerated plants.

After confirming OsALS-P171 site could work well as a selectable marker, other three sites—OsAccase W2125, OsBDAH2 Stop and OsSbe2 Stop, were combined with this selectable site separately. Both biolistic and Agrobacterium-mediated delivery were used for transformation. Surviving seedling showed target site mutation.

### Example 12

### Development of Base Co-Editing System in Corn Based on ZmALS-P197 or ZmALS-G654

1. Generation of Amino Acid Conversions that Confer Herbicide Resistance Made with Base Editors

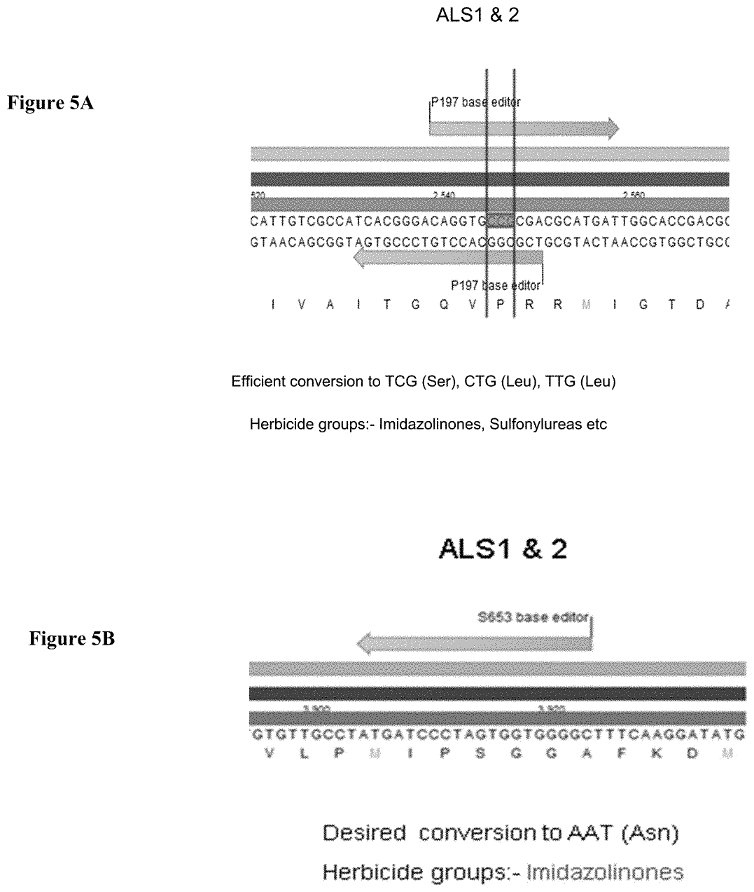

Target amino acids of Zea mays were chosen for conversion to amino acids that have been seen in weeds resistant to the herbicide groups like imidazolinones and sulfonylureas. The green arrows in FIG. 5 are guide sequences to the coding or non-coding strand for obtaining desired conversion. Note: coordinates of the amino acid residues numbered in this Example are standardized to the archetypal ALS gene from Arabidopsis thaliana. Positions of these residues in the corn and wheat peptide sequences will be somewhat different.

2. The Herbicide-Sensitive P197 Codon in Corn ALS can be Efficiently Edited by Base Editors

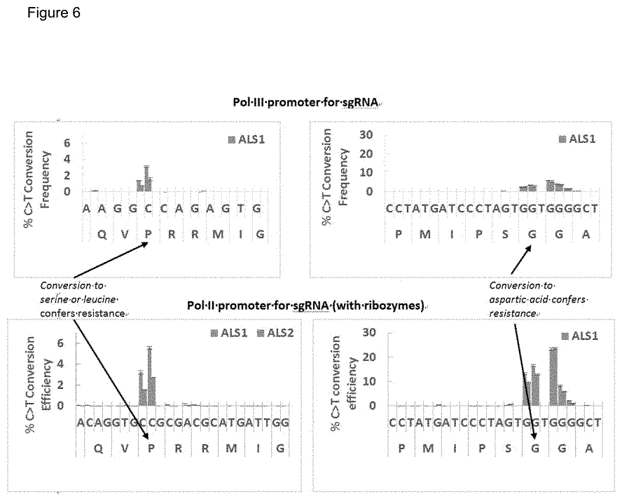

All experiments carried out in Corn Protoplast system. Pol III promoter for sgRNA:—Guides were made to modify ALS1 and ALS2 genes at the P197 locus (FIG. 6, left graph , top) and G654 locus (FIG. 6, Right graph, top). The base-editor system is a single vector system, in this case with a pUbi1 driven base editor and a ZmU3-driven guide RNA. The results shown above is a % C to T conversion frequency calculated for every C in the guide RNA and minus the background from the negative control for both ALS1 and ALS2. The frequency shown here does not specify whether one or both C's at P197 codon changed in the same cell. At the G654 locus the changes were also evident but to a lesser extent.

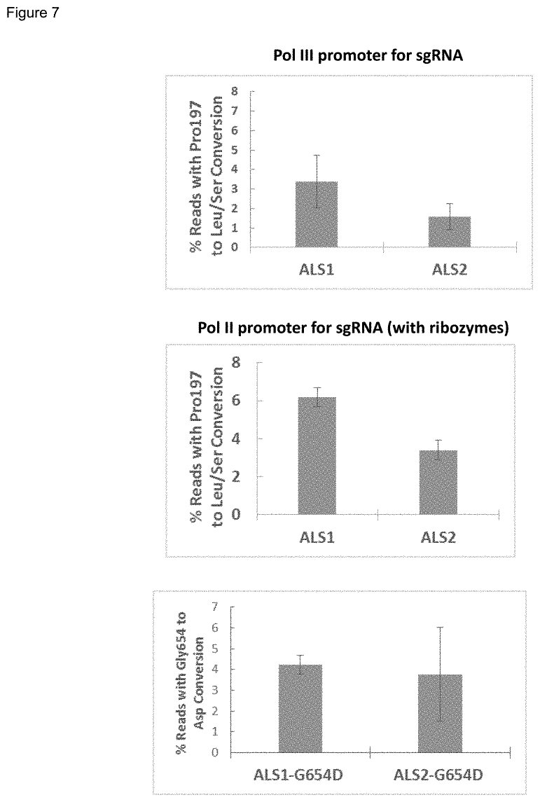

3. The Herbicide-Sensitive Residue is Converted to Herbicide-Resistant up to 6% Frequency of Treated Cells (FIG. 7)

Another way of analyzing the data shown in FIG. 6 is by counting the number of reads which show the desired amino acid codon conversion. The final % data is normalized to the protoplast transformation efficiency.

Top panel:—Shows the % of reads where the proline197 has been converted to a Leucine or a Serine at both ALS1 and ALS2 loci. The data is from experiment where the Pol III promoter was used.

Middle Panel:—Shows the % of reads where the proline197 has been converted to a Leucine or a Serine at both ALS1 and ALS2 loci. The data is from experiment where the Pol II promoter and Ribozyme delivery strategy for sgRNA was used.

Bottom Panel:—Shows the % of reads where the Glycine654 has been converted to an Aspartic Acid at both ALS 1 and ALS2 loci. The data is from experiment where the Pol III promoter and Ribozyme delivery strategy for sgRNA was used.

1. A method for isolating at least one modified plant cell or at least one modified plant tissue, organ, or whole plant comprising the at least one modified plant cell, without stably integrating a transgenic selectable marker sequence, the method comprising:
a. introducing at least one first targeted base modification or at least one first targeted frameshift or deletion modification, into a first plant genomic target site of at least one plant cell to be modified, wherein the at least one targeted base modification causes expression of at least one phenotypically selectable trait, and wherein the at least one first targeted frameshift or deletion modification causes expression of at least one phenotypically selectable trait;
b. introducing at least one second targeted modification into a second plant genomic target site of the at least one plant cell to be modified, wherein the at least one second targeted modification is introduced using at least one of a site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and
c. isolating at least one modified plant cell, tissue, organ, or whole plant, or isolating at least one progeny cell, tissue, organ, or plant thereof by selecting
i. for the at least one phenotypically selectable trait caused by the at least one first targeted base modification at the first plant genomic target site, and optionally by further selecting
ii. for the at least one second targeted modification in the second plant genomic target site.

2. The method of claim 1, wherein step b. additionally comprises introducing a repair template to make a targeted sequence conversion or replacement at the at least second plant genomic target site.

3. The method of claim 1, comprising a further step of
d. crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

4. The method of claim 1, wherein the at least one site-specific effector is temporarily or permanently linked to at least one base editing complex, wherein the base editing complex mediates the at least one first targeted base modification of step a.

5. The method of claim 1, wherein the at least one site-specific effector is selected from at least one of a nuclease, comprising a CRISPR nuclease, including Cas or Cpf1 nucleases, a TALEN, a ZFN, a meganuclease, an Argonaute nuclease, a restriction endonuclease, including FokI or a variant thereof, a recombinase, or two site-specific nicking endonucleases, or a base editor, or any variant or catalytically active fragment of the aforementioned effectors.

6. (canceled)

7. The method of claim 1, wherein the at least one first targeted base modification is made by at least one base editing complex comprising at least one base editor as component.

8. The method of claim 7, wherein the base editing complex comprises at least one cytidine deaminase, or a catalytically active fragment thereof.

9. (canceled)

10. The method of claim 7, wherein the base editing complex contains an APOBEC1 component.

11-14. (canceled)

15. The method of claim 7, wherein at least one component of the at least one base editing complex comprises at least one organelle localization signal to target the at least one base editing complex to a subcellular organelle.

16-18. (canceled)

19. The method of claim 1, wherein the first plant genomic target site of the at least one plant cell is a genomic target site encoding at least one phenotypically selectable trait, wherein the at least one phenotypically selectable trait is a resistance/tolerance trait or a growth advantage trait, and wherein the at least one first targeted base modification at the first plant genomic target site of the at least one plant cell confers resistance/tolerance or a growth advantage towards a compound or trigger to be added to the at least one modified plant cell, tissue or plant, or a progeny thereof.

20-41. (canceled)

42. The method of claim 1, wherein the at least one phenotypically selectable trait is a visible phenotype that is useful in identifying or isolating at least one modified plant cell, tissue, organ or whole plant.

43-45. (canceled)

46. The method of claim 1, wherein the at least one plant cell to be modified is preferably being derived from a plant selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

47. A method for isolating at least one modified plant cell or at least one modified plant tissue, organ, or whole plant comprising the at least one modified plant cell, without stably integrating a transgenic selectable marker sequence, the method comprising:
a. introducing at least one first targeted codon deletion modification into a first plant genomic target site of at least one plant cell to be modified using at least one first site-specific effector, comprising a nuclease, a recombinase, or a DNA modification reagent, wherein the at least one targeted codon deletion modification causes expression of at least one phenotypically selectable trait;
b. introducing at least one second targeted modification into a second plant genomic target site of the at least one plant cell to be modified, wherein the at least one second targeted modification is introduced using at least one second site-specific effector to create the at least one second targeted modification at the second plant genomic target site, wherein the at least one second targeted modification is introduced simultaneously or subsequently to the introduction of the at least one first targeted base modification into the same at least one plant cell to be modified, or into at least one progeny cell, tissue, organ, or plant thereof comprising the at least one first targeted modification to obtain at least one modified plant cell; and
c. isolating at least one modified plant cell, tissue, organ, or whole plant, or isolating at least one progeny cell, tissue, organ, or plant thereof by selecting
i. for the at least one phenotypically selectable trait caused by the at least one first targeted codon deletion modification at the first plant genomic target site, and optionally by further selecting
ii. for the at least one second targeted modification in the second plant genomic target site,
d. optionally: crossing at least one modified plant or plant material comprising the at least one first and the at least one second targeted modification with a further plant or plant material of interest to segregate the resulting progeny plants or plant material to achieve a genotype of interest, optionally wherein the genotype of interest does not comprise the at least one first targeted modification.

48-50. (canceled)

51. The method of claim 47, wherein the at least site-specific effector, or at least one component of a complex comprising the at least one site-specific effector, comprises at least one organelle localization signal to target the at least one base editing complex to a subcellular organelle.

52-57. (canceled)

58. The method of claim 47, wherein the at least one phenotypically selectable trait is a phytotoxic resistance/tolerance trait, preferably a herbicide resistance/tolerance trait, and wherein the at least one first targeted codon deletion modification at the first plant genomic target site of the at least one plant cell to be modified confers resistance/tolerance for a phytotoxic compound, preferably a herbicide, said compound being an exogenous compound to be added to the at least one modified plant cell, tissue, organ, or whole plant, or a progeny thereof.

59-61. (canceled)

62. The method of claim 47, wherein the at least one plant cell to be modified is preferably being derived from a plant selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

63-66. (canceled)

67. The method of claim 47, wherein the at least site-specific effector, or at least one component of a complex comprising the at least one site-specific effector, comprises at least one organelle localization signal to target the at least one base editing complex to a subcellular organelle.

68-79. (canceled)

80. The method of claim 47, wherein the at least one plant cell to be modified is preferably derived from a plant selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

81. A plant cell, tissue, organ, material or whole plant, or a progeny thereof, obtainable by a method according to claim 1.

82. A plant cell, tissue, organ, material or whole plant, or a progeny thereof, obtainable by a method according to claim 47.

83. (canceled)

84. A method of generating a genetically modified plant by genome editing, the method comprising the steps of:
a) providing cells or tissues of the plant to be genetically modified;
b) providing a first genome editing system and a second genome editing system, wherein the first genome editing system can target and modify a plant selectable marker gene, and the second genome modification system can target and modify a gene of interest in the plant;
c) co-transforming the cells or tissues with the first and second genome editing systems;
d) regeneration plants from said transformed cells or tissues, preferably without selection pressure;
e) selecting plants in which the selectable marker gene has been modified from the plants regenerated in step d); and
f) identifying a plant whose target gene is modified from the plants selected in step e).

85. (canceled)

86. The method of claim 84, wherein the modification of the selectable marker gene results in a selectable trait in the plant, preferably, the modification of the selectable marker gene does not alter the other traits of the plant.

87. The method of claim 86, wherein the selectable trait is herbicide resistance.

88. The method of claim 87, wherein the selectable marker gene is selected from the group consisting of sbA, ALS, EPSPS, ACCase, PPO, HPPD, PDS, GS, DOXPS, and P450.

89-91. (canceled)

92. The method of claim 84, wherein the plant is selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

93. (canceled)


SEQUENCE LISTING (28 sequences)

SEQ ID NO: 1 - DNA, 5142 bp, Artificial Sequence
  APOBEC1 XTEN nCas9(D10A) UGI NLS construct
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaagacgtctaa

SEQ ID NO: 2 - DNA, 5214 bp, Artificial Sequence
  APOBEC1 XTEN nCas9(D10A) UGI NLS construct codon optimized
atgccaaagaagaagaggaaggtttcatcggagaccggccctgttgctgttgaccccaccctgcggcggagaatcgagccacacgagttcgaggtgttcttcgacccaagggagctccgcaaggaaacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgaaacgccaggcacctccgagagcgctacgcctgaacttaaggacaagaagtactcgatcggcctcgccatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgatgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtttccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgaaacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgaaacgaggatcgacctctcacaactcggcggggataagagacccgcagcaaccaagaaggcagggcaagcaaagaagaagaagacgcgtgactccggcggcagcaccaacctgtccgacatcatcgagaaggaaacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtcgggggggagcccaaagaagaagcggaaggtgtag

SEQ ID NO: 3 - DNA, 20 bp, Artificial Sequence
  Protospacer sequence
caggtgccgcgacgcatgat

SEQ ID NO: 4 - DNA, 21 bp, Artificial Sequence
  Protospacer sequence
cacgggacaggtgccgcgacg

SEQ ID NO: 5 - DNA, 20 bp, Artificial Sequence
  Protospacer sequence
gggacaggtgccgcgacgca

SEQ ID NO: 6 - DNA, 20 bp, Artificial Sequence
  Protospacer sequence
gccccaccactagggatcat

SEQ ID NO: 7 - DNA, 20 bp, Artificial Sequence
  Protospacer sequence
atcaccagcatagacacctt

SEQ ID NO: 8 - DNA, 21 bp, Artificial Sequence
  Protospacer sequence
aggatcaccagcatagacacc

SEQ ID NO: 9 - DNA, 20 bp, Artificial Sequence
  Protospacer sequence
cttagaaggatcaccagcat

SEQ ID NO: 10 - DNA, 20 bp, Artificial Sequence
  Protospacer sequence
ctgagcagaaaggctcaatg

SEQ ID NO: 11 - DNA, 21 bp, Artificial Sequence
  Protospacer sequence
ataagcacctgagcagaaagg

SEQ ID NO: 12 - DNA, 21 bp, Artificial Sequence
  Protospacer sequence
cctttctgctcaggtgcttat

SEQ ID NO: 13 - DNA, 20 bp, Artificial Sequence
  Protospacer sequence
acctgagcagaaaggctcaa

SEQ ID NO: 14 - DNA, 4047 bp, Artificial Sequence
  APOBEC1 XTEN linker CasX1 UGI NLS codon optimized
atgccaaagaagaagaggaaggtttcatcggagaccggccctgttgctgttgaccccaccctgcggcggagaatcgagccacacgagttcgaggtgttcttcgacccaagggagctccgcaaggaaacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgaaacgccaggcacctccgagagcgctacgcctgaacttaaggagaagagaattaacaagatcagaaaaaaattgagcgccgacaatgcgactaaaccagtttccagaagcggccctatgaaaacgctcctcgtgcgggtcatgacagatgaccttaaaaaacgccttgagaagcgcagaaagaaaccggaagtgatgcctcaagttatttccaataatgccgccaataacctccgcatgcttttggatgactacaccaaaatgaaggaagcgatacttcaagtttactggcaagagttcaaagatgatcacgttggtcttatgtgtaaatttgcccaaccggcctctaagaagatagatcagaacaagctgaagccagagatggacgagaagggaaatctcacgactgcgggcttcgcgtgctcgcaatgtggtcagcctctctttgtgtataaacttgagcaagtctcagagaaggggaaagcatatacgaactacttcggtagatgcaacgtggcagagcatgaaaaacttattttgctcgctcagctgaaaccggagaaagactcggacgaagcagttacttatagccttggcaaatttggccaaagggcactcgacttctatagcatccacgtgacgaaggaatctacgcatccagtgaaaccattggcgcagattgcaggaaatcgctatgcgtcgggaccggtgggcaaggccctttcggatgcctgtatgggtacgatagcttcctttttgtcaaagtaccaagatataattatcgaacaccaaaaggtcgtcaaggggaatcaaaagagattggaaagtttgagggagctcgctggcaaggagaatctcgaatatccatcagtcacgctccctccgcagccacataccaaggaaggggttgacgcttataatgaggttatcgcgcgggtccgcatgtgggtcaacttgaatctttggcaaaaactcaaactgtccagagatgatgcaaagcctttgctcaggttgaagggcttcccttcgttcccagtcgttgaaaggagagaaaacgaagtcgattggtggaacactatcaatgaagtgaaaaagctcattgatgctaagagagacatgggtagggtcttttggtctggagttaccgcagaaaagcggaatactattctggaaggctacaactatcttcccaacgaaaacgaccacaagaaaagggaggggagcctcgaaaatcccaaaaaaccggcgaaacgccaatttggggatctgcttctttatctggagaagaagtatgcaggcgactggggaaaagtgtttgacgaggcttgggagcgcatcgacaaaaagatcgctggcctcacatcacacatagaaagggaggaggcaaggaatgcagaagatgcgcagagcaaagcagttcttacggattggttgcgcgctaaggcttcctttgttttggagcgcttgaaggaaatggacgaaaaggaattttatgcgtgcgaaatccagctgcaaaaatggtatggtgatttgagggggaaccccttcgctgtggaagccgaaaaccgggtcgtggacatatccgggttttccatagggtcggacggtcactccattcaataccggaatttgcttgcatggaaatatcttgagaacggtaagcgggagttttatttgctgatgaactacggaaaaaagggtcgcattaggttcactgatggcacagatattaaaaaaagcggtaagtggcaaggtcttctgtacggcggaggaaaggcgaaggttatcgacttgacctttgacccagacgatgagcagttgattattttgcctttggcattcggtacaagacaagggagggaattcatctggaacgatctgctctcccttgaaacgggtctcatcaagctggctaacggcagagtcatagagaaaaccatatataataagaagattggtagagatgagccggctctttttgtggcgctcactttcgagaggcgcgaggtcgttgacccgtccaacatcaagcccgttaacctgatcggtgttgataggggagaaaacataccggcggtgatagcacttaccgacccagagggatgccccctcccagaattcaaagattcttcggggggaccaactgacattctcaggataggtgagggctataaggagaagcagcgcgctatccaagcggcgaaggaagtcgagcaacggagagcggggggctattctcggaaattcgcatcgaaaagccggaatcttgccgacgacatggtcaggaactcagccagggacctcttctatcacgcggttacgcacgacgccgttcttgtttttgaaaatctctcgcggggttttggacggcaaggtaagcggacctttatgacggaaagacagtacaccaaaatggaagattggctcaccgcgaagctcgcgtacgaggggcttacatctaaaacgtacttgtccaaaacactcgcccagtacactagcaaaacgtgttctaactgcggctttacgatcactaccgcggactacgacggcatgctcgtcaggctcaagaaaacgtctgacggatgggcaaccacacttaacaataaagagctcaaggctgaaggtcagatcacatattataatagatataagaggcagaccgtggagaaggagctgtcagctgagcttgacaggttgtctgaggagtccggcaacaacgatatttctaagtggacaaaaggacggagagatgaagcattgtttctgctcaaaaagcggttctcgcacaggcccgttcaggagcagtttgtttgtcttgattgcggtcacgaggtccacgcggatgagcaggccgctctcaatatagcgaggagctggttgtttttgaactctaattccacagaattcaaaagctataagtccgggaagcaaccgttcgtgggcgcttggcaagccttttataagcgcaggctcaaggaggtttggaaaccaaacgctaaacgccccgcggctacaaagaaggctggccaggcaaagaagaagaagaccaacctgtccgacatcatcgagaaggaaacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtcgggggggagcccaaagaagaagcggaaggtgtag

SEQ ID NO: 15 - DNA, 4962 bp, Artificial Sequence
  APOBEC1 XTEN linker AsCpf1(R1226A) UGI NLS codon optimized
atgccaaagaagaagaggaaggtttcatcggagaccggccctgttgctgttgaccccaccctgcggcggagaatcgagccacacgagttcgaggtgttcttcgacccaagggagctccgcaaggaaacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgaaacgccaggcacctccgagagcgctacgcctgaacttaagacccaatttgagggatttacgaatctttatcaagtttcaaagacgcttaggtttgagctcattccacaaggaaaaaccttgaagcacattcaagagcagggctttatcgaggaagacaaggcacggaatgaccattataaagaattgaaacccataatcgatcgcatatacaaaacttatgccgaccaatgcttgcagcttgtccaactcgactgggaaaatctctcggctgcgatagactcttacaggaaggaaaagacagaagaaacaagaaacgccctcattgaagagcaggctacgtatagaaatgctattcacgactatttcattggcagaacagataacttgacggacgccataaacaaaagacatgcggagatctacaagggattgttcaaagcggagcttttcaacggaaaagttctcaagcagcttggcacggtcaccactaccgaacacgaaaacgccttgttgaggagcttcgataagttcacgacatatttctctggtttctatgagaatcggaagaatgtcttctctgcagaagacatttcaaccgcaatcccacaccggattgtgcaagataactttccgaaatttaaggaaaactgtcacatcttcactaggttgattacggctgttccatctcttagagaacacttcgaaaacgtcaaaaaagctataggcattttcgtctcaacgagcatagaggaggtcttctcgttccctttctataaccagcttctcacccagacacagattgatctctataatcaactccttggtggtatttcaagggaagccgggacggagaagattaaggggttgaatgaagttctcaatctggcgatacagaagaatgacgaaaccgcccatattatagcttccctcccacatcggtttataccgttgttcaagcagatcctgtcggaccgcaacacgctttctttcatactcgaagagttcaaaagcgacgaggaagtcatacagagcttctgtaagtataaaacacttttgaggaatgaaaacgttcttgaaactgccgaggccttgtttaacgagctcaacagcatagatcttacgcatatttttatttcccacaaaaaattggaaactataagctcagcgctgtgtgatcactgggatacgcttcgcaatgccctttatgagcgcaggatcagcgaactgacggggaagattacgaaatctgcgaaagagaaagttcaaaggtcccttaagcacgaggatattaatctccaagaaataataagcgcggctggtaaagaactttccgaagctttcaagcaaaagacatccgaaatactctcccatgcgcatgcagccctggaccaaccattgccaacaactttgaagaaacaagaagagaaggaaatcctgaagtcccaactcgactctttgctcggcctctatcacttgcttgattggttcgcggttgatgagtccaacgaagttgaccctgagttcagcgccaggttgaccggtataaagttggaaatggaaccaagcctctcattttacaacaaggcgaggaactacgcgaccaagaaaccatacagcgtcgaaaagtttaagcttaactttcaaatgccaacgctcgcttccggttgggatgttaacaaagaaaaaaataacggcgccatcttgtttgttaaaaacggtttgtattacctcggcatcatgccaaaacaaaagggtcggtacaaggctctgagcttcgagccaacagagaaaacaagcgaaggcttcgacaagatgtattatgattactttcccgatgcagctaaaatgatccccaagtgctcaacacagcttaaagcggttaccgcccatttccagactcacacgaccccaattctcttgtcaaataactttattgaacccttggaaataaccaaagagatatatgaccttaataacccggagaaagaacccaagaagttccagacggcgtacgctaagaaaacaggagatcagaagggctatagggaggccctttgtaaatggattgactttacaagggactttttgtcgaaatatacgaagaccacttcaattgacctttcgtccctgcggccgtctagccagtataaagatttgggtgagtactatgcggaacttaatcctttgttgtaccacatatcttttcaacggattgcagagaaggagataatggatgcggtcgaaacaggaaagctctatctgttccagatttacaataaagattttgccaagggacaccatggaaaacctaacctgcatactctttactggacgggtcttttctcgccggaaaatttggctaagacgtctatcaagttgaatgggcaggcagaactcttctatcgccctaagtctaggatgaaacggatggctcatcggctgggtgaaaaaatgctcaacaaaaagcttaaggatcaaaagacaccaatcccggacacactttatcaagaattgtacgattacgttaatcacagactctcacatgacctttcagatgaggcccgcgctttgcttcccaatgttattactaaagaggtctcgcatgagatcataaaagatagaagattcacgtctgataagttcttttttcatgtgccaataactctcaactatcaggccgcaaattcgccgtccaagttcaaccaaagggtgaatgcctacctcaaggagcacccggagacgccaataataggtatcgatcggggcgaacgcaaccttatttatataacagttatcgatagcacagggaaaatactggagcagcggagcctgaatactattcaacagtttgactaccaaaagaaactggacaatagagagaaggagcgcgtcgccgcccggcaagcttggtccgtggtcggaactataaaagatcttaaacagggatacctgtcacaggtcatccatgaaatcgtggatctgatgatacactatcaagctgttgtcgtgctcgaaaacttgaattttggattcaaatcgaagagaactggaatcgctgaaaaagcggtgtaccaacagttcgagaagatgctcatcgataagcttaattgtttggtgcttaaggactatcccgccgaaaaggttgggggggtgctgaacccgtatcagctcacagatcaatttacttcattcgcgaagatgggaacgcagtcaggatttctgttctacgttccagccccttatacgtcgaaaattgaccctcttacggggttcgtggacccctttgtttggaaaacgataaaaaaccacgagtcacgcaagcactttctcgagggatttgattttcttcattatgatgtgaagaccggggacttcattttgcactttaagatgaacaggaacttgtctttccaaaggggcttgcctggattcatgccggcctgggatatcgtgtttgaaaagaacgaaacacagttcgatgcgaaagggacgcccttcatagctggaaagcgcatagttccagtgattgagaaccacagattcactggtcgctacagagacctgtatccggcaaatgaactgatagcactccttgaggaaaagggtatcgtgtttcgcgatggttcaaatattctcccgaagcttttggagaacgacgattctcatgctatagatactatggtcgctctcatccggtccgtccttcaaatggccaattcgaatgcagcgaccggtgaggattacataaattcaccagtccgggaccttaatggggtttgcttcgactcgcgctttcaaaaccccgaatggccaatggacgccgatgctaacggtgcctaccatatagcacttaaaggacagcttctgttgaatcaccttaaagaatcaaaagaccttaagctgcagaatggaatttcaaatcaggattggctcgcgtacatacaggagcttcgcaataccaacctgtccgacatcatcgagaaggaaacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtcgggggggagcccaaagaagaagcggaaggtgtag

SEQ ID NO: 16 - DNA, 5121 bp, Artificial Sequence
  NLS dCas9 NLS Linker PmCDA1 UGI construct
atgccaaagaagaagaggaaggttgacaagaagtactcgatcggcctcgccatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgatgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtttccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgaaacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgaaacgaggatcgacctctcacaactcggcggggataagagacccgcagcaaccaagaaggcagggcaagcaaagaagaagaagacgcgtgactccggcggcagcccaaagaagaagaggaaggttggtggaggaggttctggaggtggaggttctatgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgttaccaacctgtccgacatcatcgagaaggaaacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtga

SEQ ID NO: 17 - DNA, 4101 bp, Artificial Sequence
  nCas9 (D10A)
gataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 18 - DNA, 3003 bp, Artificial Sequence
  CasX
gagaagagaattaacaagatcagaaaaaaattgagcgccgacaatgcgactaaaccagtttccagaagcggccctatgaaaacgctcctcgtgcgggtcatgacagatgaccttaaaaaacgccttgagaagcgcagaaagaaaccggaagtgatgcctcaagttatttccaataatgccgccaataacctccgcatgcttttggatgactacaccaaaatgaaggaagcgatacttcaagtttactggcaagagttcaaagatgatcacgttggtcttatgtgtaaatttgcccaaccggcctctaagaagatagatcagaacaagctgaagccagagatggacgagaagggaaatctcacgactgcgggcttcgcgtgctcgcaatgtggtcagcctctctttgtgtataaacttgagcaagtctcagagaaggggaaagcatatacgaactacttcggtagatgcaacgtggcagagcatgaaaaacttattttgctcgctcagctgaaaccggagaaagactcggacgaagcagttacttatagccttggcaaatttggccaaagggcactcgacttctatagcatccacgtgacgaaggaatctacgcatccagtgaaaccattggcgcagattgcaggaaatcgctatgcgtcgggaccggtgggcaaggccctttcggatgcctgtatgggtacgatagcttcctttttgtcaaagtaccaagatataattatcgaacaccaaaaggtcgtcaaggggaatcaaaagagattggaaagtttgagggagctcgctggcaaggagaatctcgaatatccatcagtcacgctccctccgcagccacataccaaggaaggggttgacgcttataatgaggttatcgcgcgggtccgcatgtgggtcaacttgaatctttggcaaaaactcaaactgtccagagatgatgcaaagcctttgctcaggttgaagggcttcccttcgttcccagtcgttgaaaggagagaaaacgaagtcgattggtggaacactatcaatgaagtgaaaaagctcattgatgctaagagagacatgggtagggtcttttggtctggagttaccgcagaaaagcggaatactattctggaaggctacaactatcttcccaacgaaaacgaccacaagaaaagggaggggagcctcgaaaatcccaaaaaaccggcgaaacgccaatttggggatctgcttctttatctggagaagaagtatgcaggcgactggggaaaagtgtttgacgaggcttgggagcgcatcgacaaaaagatcgctggcctcacatcacacatagaaagggaggaggcaaggaatgcagaagatgcgcagagcaaagcagttcttacggattggttgcgcgctaaggcttcctttgttttggagcgcttgaaggaaatggacgaaaaggaattttatgcgtgcgaaatccagctgcaaaaatggtatggtgatttgagggggaaccccttcgctgtggaagccgaaaaccgggtcgtggacatatccgggttttccatagggtcggacggtcactccattcaataccggaatttgcttgcatggaaatatcttgagaacggtaagcgggagttttatttgctgatgaactacggaaaaaagggtcgcattaggttcactgatggcacagatattaaaaaaagcggtaagtggcaaggtcttctgtacggcggaggaaaggcgaaggttatcgacttgacctttgacccagacgatgagcagttgattattttgcctttggcattcggtacaagacaagggagggaattcatctggaacgatctgctctcccttgaaacgggtctcatcaagctggctaacggcagagtcatagagaaaaccatatataataagaagattggtagagatgagccggctctttttgtggcgctcactttcgagaggcgcgaggtcgttgacccgtccaacatcaagcccgttaacctgatcggtgttgataggggagaaaacataccggcggtgatagcacttaccgacccagagggatgccccctcccagaattcaaagattcttcggggggaccaactgacattctcaggataggtgagggctataaggagaagcagcgcgctatccaagcggcgaaggaagtcgagcaacggagagcggggggctattctcggaaattcgcatcgaaaagccggaatcttgccgacgacatggtcaggaactcagccagggacctcttctatcacgcggttacgcacgacgccgttcttgtttttgaaaatctctcgcggggttttggacggcaaggtaagcggacctttatgacggaaagacagtacaccaaaatggaagattggctcaccgcgaagctcgcgtacgaggggcttacatctaaaacgtacttgtccaaaacactcgcccagtacactagcaaaacgtgttctaactgcggctttacgatcactaccgcggactacgacggcatgctcgtcaggctcaagaaaacgtctgacggatgggcaaccacacttaacaataaagagctcaaggctgaaggtcagatcacatattataatagatataagaggcagaccgtggagaaggagctgtcagctgagcttgacaggttgtctgaggagtccggcaacaacgatatttctaagtggacaaaaggacggagagatgaagcattgtttctgctcaaaaagcggttctcgcacaggcccgttcaggagcagtttgtttgtcttgattgcggtcacgaggtccacgcggatgagcaggccgctctcaatatagcgaggagctggttgtttttgaactctaattccacagaattcaaaagctataagtccgggaagcaaccgttcgtgggcgcttggcaagccttttataagcgcaggctcaaggaggtttggaaaccaaacgctaaacgccccgcggctacaaagaaggctggccaggcaaagaagaagaag

SEQ ID NO: 19 - DNA, 3918 bp, Artificial Sequence
  AsCpf1 (R1226A)
acccaatttgagggatttacgaatctttatcaagtttcaaagacgcttaggtttgagctcattccacaaggaaaaaccttgaagcacattcaagagcagggctttatcgaggaagacaaggcacggaatgaccattataaagaattgaaacccataatcgatcgcatatacaaaacttatgccgaccaatgcttgcagcttgtccaactcgactgggaaaatctctcggctgcgatagactcttacaggaaggaaaagacagaagaaacaagaaacgccctcattgaagagcaggctacgtatagaaatgctattcacgactatttcattggcagaacagataacttgacggacgccataaacaaaagacatgcggagatctacaagggattgttcaaagcggagcttttcaacggaaaagttctcaagcagcttggcacggtcaccactaccgaacacgaaaacgccttgttgaggagcttcgataagttcacgacatatttctctggtttctatgagaatcggaagaatgtcttctctgcagaagacatttcaaccgcaatcccacaccggattgtgcaagataactttccgaaatttaaggaaaactgtcacatcttcactaggttgattacggctgttccatctcttagagaacacttcgaaaacgtcaaaaaagctataggcattttcgtctcaacgagcatagaggaggtcttctcgttccctttctataaccagcttctcacccagacacagattgatctctataatcaactccttggtggtatttcaagggaagccgggacggagaagattaaggggttgaatgaagttctcaatctggcgatacagaagaatgacgaaaccgcccatattatagcttccctcccacatcggtttataccgttgttcaagcagatcctgtcggaccgcaacacgctttctttcatactcgaagagttcaaaagcgacgaggaagtcatacagagcttctgtaagtataaaacacttttgaggaatgaaaacgttcttgaaactgccgaggccttgtttaacgagctcaacagcatagatcttacgcatatttttatttcccacaaaaaattggaaactataagctcagcgctgtgtgatcactgggatacgcttcgcaatgccctttatgagcgcaggatcagcgaactgacggggaagattacgaaatctgcgaaagagaaagttcaaaggtcccttaagcacgaggatattaatctccaagaaataataagcgcggctggtaaagaactttccgaagctttcaagcaaaagacatccgaaatactctcccatgcgcatgcagccctggaccaaccattgccaacaactttgaagaaacaagaagagaaggaaatcctgaagtcccaactcgactctttgctcggcctctatcacttgcttgattggttcgcggttgatgagtccaacgaagttgaccctgagttcagcgccaggttgaccggtataaagttggaaatggaaccaagcctctcattttacaacaaggcgaggaactacgcgaccaagaaaccatacagcgtcgaaaagtttaagcttaactttcaaatgccaacgctcgcttccggttgggatgttaacaaagaaaaaaataacggcgccatcttgtttgttaaaaacggtttgtattacctcggcatcatgccaaaacaaaagggtcggtacaaggctctgagcttcgagccaacagagaaaacaagcgaaggcttcgacaagatgtattatgattactttcccgatgcagctaaaatgatccccaagtgctcaacacagcttaaagcggttaccgcccatttccagactcacacgaccccaattctcttgtcaaataactttattgaacccttggaaataaccaaagagatatatgaccttaataacccggagaaagaacccaagaagttccagacggcgtacgctaagaaaacaggagatcagaagggctatagggaggccctttgtaaatggattgactttacaagggactttttgtcgaaatatacgaagaccacttcaattgacctttcgtccctgcggccgtctagccagtataaagatttgggtgagtactatgcggaacttaatcctttgttgtaccacatatcttttcaacggattgcagagaaggagataatggatgcggtcgaaacaggaaagctctatctgttccagatttacaataaagattttgccaagggacaccatggaaaacctaacctgcatactctttactggacgggtcttttctcgccggaaaatttggctaagacgtctatcaagttgaatgggcaggcagaactcttctatcgccctaagtctaggatgaaacggatggctcatcggctgggtgaaaaaatgctcaacaaaaagcttaaggatcaaaagacaccaatcccggacacactttatcaagaattgtacgattacgttaatcacagactctcacatgacctttcagatgaggcccgcgctttgcttcccaatgttattactaaagaggtctcgcatgagatcataaaagatagaagattcacgtctgataagttcttttttcatgtgccaataactctcaactatcaggccgcaaattcgccgtccaagttcaaccaaagggtgaatgcctacctcaaggagcacccggagacgccaataataggtatcgatcggggcgaacgcaaccttatttatataacagttatcgatagcacagggaaaatactggagcagcggagcctgaatactattcaacagtttgactaccaaaagaaactggacaatagagagaaggagcgcgtcgccgcccggcaagcttggtccgtggtcggaactataaaagatcttaaacagggatacctgtcacaggtcatccatgaaatcgtggatctgatgatacactatcaagctgttgtcgtgctcgaaaacttgaattttggattcaaatcgaagagaactggaatcgctgaaaaagcggtgtaccaacagttcgagaagatgctcatcgataagcttaattgtttggtgcttaaggactatcccgccgaaaaggttgggggggtgctgaacccgtatcagctcacagatcaatttacttcattcgcgaagatgggaacgcagtcaggatttctgttctacgttccagccccttatacgtcgaaaattgaccctcttacggggttcgtggacccctttgtttggaaaacgataaaaaaccacgagtcacgcaagcactttctcgagggatttgattttcttcattatgatgtgaagaccggggacttcattttgcactttaagatgaacaggaacttgtctttccaaaggggcttgcctggattcatgccggcctgggatatcgtgtttgaaaagaacgaaacacagttcgatgcgaaagggacgcccttcatagctggaaagcgcatagttccagtgattgagaaccacagattcactggtcgctacagagacctgtatccggcaaatgaactgatagcactccttgaggaaaagggtatcgtgtttcgcgatggttcaaatattctcccgaagcttttggagaacgacgattctcatgctatagatactatggtcgctctcatccggtccgtccttcaaatggccaattcgaatgcagcgaccggtgaggattacataaattcaccagtccgggaccttaatggggtttgcttcgactcgcgctttcaaaaccccgaatggccaatggacgccgatgctaacggtgcctaccatatagcacttaaaggacagcttctgttgaatcaccttaaagaatcaaaagaccttaagctgcagaatggaatttcaaatcaggattggctcgcgtacatacaggagcttcgcaat

SEQ ID NO: 20 - DNA, 687 bp, Artificial Sequence
  APOBEC1
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 21 - DNA, 249 bp, Artificial Sequence
  UGI
accaacctgtccgacatcatcgagaaggaaacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctg

SEQ ID NO: 22 - DNA, 624 bp, Artificial Sequence
  PmCDA1
atgaccgacgctgagtacgtgagaatccatgagaagttggacatctacacgtttaagaaacagtttttcaacaacaaaaaatccgtgtcgcatagatgctacgttctctttgaattaaaacgacggggtgaacgtagagcgtgtttttggggctatgctgtgaataaaccacagagcgggacagaacgtggcattcacgccgaaatctttagcattagaaaagtcgaagaatacctgcgcgacaaccccggacaattcacgataaattggtactcatcctggagtccttgtgcagattgcgctgaaaagatcttagaatggtataaccaggagctgcgggggaacggccacactttgaaaatctgggcttgcaaactctattacgagaaaaatgcgaggaatcaaattgggctgtggaacctcagagataacggggttgggttgaatgtaatggtaagtgaacactaccaatgttgcaggaaaatattcatccaatcgtcgcacaatcaattgaatgagaatagatggcttgagaagactttgaagcgagctgaaaaacgacggagcgagttgtccattatgattcaggtaaaaatactccacaccactaagagtcctgctgtt

SEQ ID NO: 23 - DNA, 20 bp, Artificial Sequence
  Protospacer sequence
tgttacttctaaactacata

SEQ ID NO: 24 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 26 - protein, 537 aa, Arabidopsis thaliana
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 27 - protein, 444 aa, Arabidopsis thaliana
KASEIVLQPIREISGLIKLPGSKSLSNRILLLAALSEGTTVVDNLLNSDDINYMLDALKRLGLNVETDSENNRAVVEGCGGIFPASIDSKSDIELYLGNAGTAMRPLTAAVTAAGGNASYVLDGVPRMRERPIGDLVVGLKQLGADVECTLGTNCPPVRVNANGGLPGGKVKLSGSISSQYLTALLMSAPLALGDVEIEIVDKLISVPYVEMTLKLMERFGVSVEHSDSWDRFFVKGGQKYKSPGNAYVEGDASSASYFLAGAAITGETVTVEGCGTTSLQGDVKFAEVLEKMGCKVSWTENSVTVTGPPRDAFGMRHLRAIDVNMNKMPDVAMTLAVVALFADGPTTIRDVASWRVKETERMIAICTELRKLGATVEEGSDYCVITPPKKVKTAEIDTYDDHRMAMAFSLAACADVPITINDPGCTRKTFPDYFQVLERITKH

SEQ ID NO: 28 - protein, 534 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA